(19) United States
(12) Patent Application Publication
(10) Pub. No.: US 2002/0199218 A1
(43) Pub. Date: Dec. 26, 2002

(54) Proline-rich extensin-like receptor kinases

(76) Inventors: Daphne Goring, Richmond Hill (CA); Nancy Silva, Mississauga (CA); Yosr Z. Haffani, Toronto (CA)

Correspondence Address: Gene J. Yao, Esquire, Synnestvedt & Lechner LLP, 2600 Aramark Tower, 1101 Market Street, Philadelphia, PA, 19107-2950, US

(21) Appl. No.: 10/086,464

(22) PCT Filed: Aug. 18, 2000
(86) PCT No.: PCT/CA00/00966

(51) Int. Cl. A01H 5/00

(52) U.S. Cl. 800/287

(57) ABSTRACT

The invention includes PERK (Proline-rich Extensin-like Receptor Kinase) nucleic acid molecules and polypeptides. A receptor-like protein kinase designated PERK1 (Proline-rich Extensin-like Receptor Kinase 1) was isolated from a (-pistil cDNA library of Brassica napus. The deduced PERK1 protein is comprised of a cytoplasmic domain that contains all of the conserved amino acids prevalent among serine/threonine kinases, a transmembrane domain and an extracellular domain with sequence similarity to the extensin family of plant cell wall proteins. Northern blot analysis demonstrated that PERK1 mRNA accumulated rapidly in leaf and stem tissue of B. napus in response to wounding and treatment with salicylic acid. In contrast, no significant accumulation of PERK1 mRNA was detected following treatment with methyl jasmonate. The rapidity of PERK1 mRNA accumulation in response to these treatments shows a role in plant defense signaling.

### CROSS REFERENCE TO RELATED APPLICATIONS

This application is a continuation-in-part of a U.S. patent application (number not yet assigned) filed on Feb. 19, 2002, which is a national phase application based on PCT/CA00/00966, filed on Aug. 18, 2000, which claims priority from U.S. patent application Ser. No. 60/149,466, filed on Aug. 19, 1999, and U.S. patent application Ser. No. 60/159,122, filed on Oct. 13, 1999, and all of the foregoing are incorporated herein by reference in their entirety.

### FIELD OF THE INVENTION

The invention relates to nucleic acid molecules and polypeptides involved in plant defense, and more particularly increasing plant resistance to wounding and pathogens. In addition, overexpression of these nucleic acids and polypeptides leads to increased growth and seed production.

### BACKGROUND OF THE INVENTION

Receptor mediated signal perception and transduction in response to external stimuli are essential for growth and developmental processes of multicellular organisms (Mu et al., 1994). These extensively well characterized processes in animal systems involve receptor protein kinase molecules comprised of an extracellular signal perception domain, a hydrophobic transmembrane domain attached to an intracellular domain that possesses kinase activity (Horn et al., 1994). In general, transmembrane signaling by receptor protein kinases requires binding of an appropriate ligand to the extracellular domain which induces receptor dimerization and alters the activity of the intracellular catalytic domain. This promotes phosphorylation of specific substrates thereby initiating a protein kinase signaling cascade (Ullrich and Schlessinger, 1990). The majority of animal receptor protein kinases isolated to date contain tyrosine-specific kinase domains (Ullrich and Schlessinger, 1990), however, the transforming growth factor beta (TGF-beta) receptor (Lin et al., 1992) and the activin receptor (Dijke et al., 1993) possess kinase domains with serine/threonine phosphorylation activity.

Intracellular communication is also essential for the growth and development of higher plants. The extensive knowledge of cell surface receptor signaling in animal systems has resulted in the isolation of several genes predicted to encode receptor-like protein kinases (RLKs). Members of the plant RLK family share highly conserved catalytic domains known to possess serine/threonine substrate specificity, yet the extracellular domains of these receptors are quite divergent which enable these proteins to selectively respond to diverse extracellular signals (Lease et al., 1998; Walker, 1994). There are several classes of plant RLKs distinguished according to characteristic amino acid sequence motifs of their extracellular domains (Shiu and Bleecker, 2001).

The S domain RLK class has a distinct extracellular domain sharing sequence identity to the S locus glycoprotein (SLG) (Stone and Walker, 1995). Among this class are the Brassica S receptor kinases (SRKs) expressed exclusively in reproductive tissues and implicated in controlling the sporophytic self-incompatibility response (Stein et al., 1991; Goring and Rothstein, 1992; Takasaki et al., 2000; Silva et al., 2001). Other RLKs of this type are represented in Arabidopsis by ARK1, ARK2, ARK3 (Tobias et al., 1992; Dwyer et al., 1994; Tobias and Nasrallah, 1996), in maize by ZmPK1 (Walker and Zhang, 1990) and by OsPK10 in rice (Zhao et al., 1994). The diverse expression patterns among members of this class suggest that these plant receptor kinases may be involved in mediating a variety of cellular signalling processes (Walker, 1994).

The largest plant RLK class is characterized by the leucine-rich repeat (LRR) motif involved in peptide ligand recognition and implicated in mediating protein-protein interactions (Shiu and Bleecker, 2001; McCarty and Chory, 2000; Braun and Walker, 1996). Analyses of Arabidopsis mutants with altered morphological phenotypes have led to the identification of several LRR RLKs with genetically defined functions. Examples include CLV1 in the regulation of floral and meristem development in Arabidopsis (Clark et al., 1997; Trotochaud et al, 2000), BRI1 in the perception and signal transduction of brassinosteroids (Li and Chory, 1997), ER in the control of organ differentiation (Torii et al., 1996), and HAESA in the control of floral organ abscission (Jinn et al, 2000). In addition, the Xa21 disease resistance gene in rice has been shown to encode a receptor kinase belonging to the LRR class (Song et al., 1995).

The lectin-like class of plant receptor kinases, represented in Arabidopsis by Ath.lecRK1 (Hervé et al., 1996) and LRK1 (Swarup et al., 1996) contain extracellular domains sharing sequence similarity with legume lectins which are known carbohydrate binding proteins and implicated in the transduction of oligosaccharide signals in plant cellular communication processes (Hervé et al., 1996). The Arabidopsis wall-associated kinase (WAKs) class is comprised of at least 22 members in the completed Arabidopsis genome and contain epidermal growth factor (EGF)-like repeats as well as limited amino acid identity to the tenascin superfamily, collagen or neurexins in their extracellular domains (Kohorn et al., 1992; He et al., 1996, 1999; Shiu and Bleecker, 2001). Recent studies have implicated WAKs in the developmental control of cell expansion and cell elongation (Wagner and Kohorn, 2001; Lally et al., 2001), in addition to the involvement of one of its members (WAK1) in mediating pathogen defense responses in Arabidopsis (He et al., 1998). WAKs are the only class of RLKs that have been demonstrated to provide transmembrane coupling between the plant cell wall and the cytosol (He et al., 1999). Other classes of plant RLKs are represented by the Arabidopsis PR5K (Wang et al., 1996), which contains an extracellular domain related to plant defense proteins, and the maize CRINKLY4 harboring a region showing similarity to the cysteine-rich repeat motif of the tumor necrosis factor receptor (TNFR) (Becraft et al., 1996). Plants remain very vulnerable to wounding and pathogens despite these advances. There is a need to identify other polypeptides that help to protect plants. There is also a need for transgenic plants which overexpress these polypeptides and which have increased resistance to wounding and pathogens.

Increased growth and seed production is a very desirable feature in crops, and while traditional breeding practices have made important gains in the past, there has been very little progress more recently. There is a need for new approaches to increase the growth and seed production in plants.

### SUMMARY OF THE INVENTION

The invention relates to nucleic acid molecules and polypeptides involved in plant defense, and more particularly increasing plant resistance to wounding and pathogens. This invention also relates to the same nucleic acid molecules and polypeptides that lead to increased growth and seed production when overexpressed in plants.

This invention also relates to nucleic acids and polypeptides that define a new class of plant RLKs with an extracellular domain rich in proline and sharing sequence similarity to the extensin family of cell wall proteins.

We isolated a cDNA clone designated PERK1 (Proline-rich Extensin-like Receptor Kinase 1) which encodes a receptor kinase in Brassica napus. We define a new class of plant receptor kinases characterized by an extracellular domain rich in proline sharing sequence similarity to the extensin family of cell wall proteins. PERK1 is induced by both wounding and chemical elicitors which mimick a pathogen attack, showing a role for PERK1 in mediating a plant's defense response to mechanical and biological attack. Overexpression of PERK1 also leads to increased growth and seed production showing a role for PERK1 in plant growth and development. Similar PERK nucleic acid molecules and polypeptides are found in other plants and cells.

The invention relates to an isolated nucleic acid molecule encoding a proline-rich, extensin-like receptor kinase (PERK) polypeptide, or a fragment of a PERK polypeptide having PERK activity. The molecule is a signaling molecule associated with the cell wall via its extensin-like extracellular domain and is involved in the transduction of extracellular stimuli into an intracellular response through a cytoplasmic kinase domain, thereby bridging the cell wall-plasma membrane continuum. The extracellular stimuli includes wounding or pathogen attack. The wounding can include a cut, a break, a tear, a fold or an insect wound. Typical pathogens include bacterial pathogens, fungal pathogens, Sclerotinia sclerotiorum, Cylindrosporium concertricum, Phoma lingam, Pseudomonas syringae, Streptomyces scabies, Blackleg, Whiterust, Fusarium Head Blight, Rust, Bunt, Leaf Spot, White mold, root rot or Fusarium ear rot. Extracellular stimuli also include signals produced by the plant during growth and development that regulate size, flowering, and seed production.

The invention also includes an isolated nucleic acid molecule encoding a PERK polypeptide, a fragment of a PERK polypeptide having PERK activity, or a polypeptide having PERK activity, comprising a nucleic acid molecule selected from the group consisting of:

(a) a nucleic acid molecule that hybridizes to a nucleic acid molecule consisting of [SEQ ID NO:1], or a complement thereof under low, moderate or high stringency hybridization conditions wherein the nucleic acid molecule encodes a PERK polypeptide or a polypeptide having PERK activity;

(b) a nucleic acid molecule degenerate with respect to (a), wherein the nucleic molecule encodes a PERK polypeptide or a polypeptide having PERK activity.

The hybridization conditions preferably include low stringency conditions of 1× SSC, 0.1% SDS at 50° C. or high stringency conditions of 0.1× SSC, 0.1% SDS at 65° C. The invention also includes an isolated nucleic acid molecule encoding a PERK polypeptide, a fragment of a PERK polypeptide having PERK activity, or a polypeptide having PERK activity, comprising a nucleic acid molecule selected from the group consisting of:

(a) the nucleic acid molecule of the coding strand shown in [SEQ ID NO:1], or a complement thereof;

(b) a nucleic acid molecule encoding the same amino acid sequence as a nucleotide sequence of (a); and

(c) a nucleic acid molecule having at least 17% identity with the nucleotide sequence of (a) and which encodes a PERK polypeptide or a polypeptide having PERK activity.

The invention also relates to an isolated nucleic acid molecule comprising a sequence that encodes a polypeptide having the sequence of SEQ ID NO:2, or the sequence of SEQ ID NO:2 with conservative amino acid substitutions.

The PERK polypeptide preferably consists of or comprises a PERK1 polypeptide.

The nucleic acid molecule preferably comprising all or part of a nucleotide sequence shown in [SEQ ID NO:1 or 3] or a complement thereof. The nucleic acid may consist of the nucleotide sequence shown in [SEQ ID NO:1 or 3] or a complement thereof. The invention also includes a PERK1 nucleic acid molecule isolated from Brassica, or a fragment thereof. The Brassica may include Brassica napus, Brassica juncea, Brassica rapa or Brassica oleracea.

Another embodiment of the invention includes a recombinant nucleic acid molecule comprising a nucleic acid molecule of the invention and a constitutive promoter sequence or an inducible promoter sequence, operatively linked so that the promoter enhances transcription of the nucleic acid molecule in a host cell.

The nucleic acid molecule of the invention optionally includes genomic DNA, cDNA or RNA. The nucleic acid molecule is optionally chemically synthesized.

The invention also includes an isolated nucleic acid molecule comprising a nucleic acid molecule selected from the group consisting of 8 to 10 nucleotides of the nucleic acid molecule of claims 6, 11 to 25 nucleotides of the nucleic acid molecule of claims 6 and 26 to 50 nucleotides of the nucleic acid molecule of claim 10. The nucleic acid molecule of the invention comprising at least 10, 15, 20, 30, 50 or 100 consecutive nucleotides of [SEQ ID NO:1 or 3] or a complement thereof. These sequences are useful as hybridization probes to detect PERK1 in a sample. They are also preferably useful as antisense oligonucleotides to inhibit gene expression. The invention also includes a nucleic acid molecule probe encoding all or part (at least 10, 15, 20, 30, 50 or 100 amino acids) of PERK1 polypeptide.

Another aspect of the invention includes a vector comprising a nucleic acid molecule of the invention. The vector optionally comprises a promoter selected from the group consisting of a super promoter, a 35S promoter of cauliflower mosaic virus, a chemical inducible promoter, a copper-inducible promoter, a steroid-inducible promoter and a tissue-specific promoter.

Another variation of the invention includes a host cell comprising the recombinant nucleic acid molecule or the vector of the invention or progeny of the host cell. The host cell of the invention is optionally selected from the group consisting of a fungal cell, a yeast cell, a bacterial cell, a microorganism cell and a plant cell. The invention includes a method of producing polypeptide, comprising culturing a host cell of the invention under conditions permitting expression of the polypeptide. The method preferably further includes isolating the protein from the cell or the cell medium.

The invention also includes a plant, a plant part, a seed, a plant cell or progeny thereof comprising a recombinant nucleic acid molecule or vector of the invention. The plant part optionally comprises all or part of a leaf, a flower, a stem, a root or a tuber. The plant, plant part, seed or plant cell is optionally selected from a species from the group consisting of Brassica napus, Brassica rapa, Brassica juncea, Brassica oleracea, or from the family Brassicaecae, Arabidopsis, potato, tomato, tobacco, cotton, carrot, petunia, sunflower, strawberries, spinach, lettuce, rice, soybean, corn, wheat, rye, barley, sorgum and alfalfa. The plant may comprise a dicot plant or a monocot plant. The invention also includes an isolated polypeptide encoded by and/or produced from a nucleic acid molecule or the vector of the invention. The polypeptide is preferably an isolated PERK polypeptide or a fragment thereof having PERK activity. The invention also includes an isolated polypeptide, the amino acid sequence of which comprises at least ten consecutive residues of [SEQ ID NO:2]. The invention also includes an isolated immunogenic polypeptide, the amino acid sequence of which comprises at least ten consecutive residues of [SEQ ID NO:2]. The invention also includes an isolated polypeptide, the amino acid sequence of which comprises residues 1 to 137, 138 to 160 and 161 to 648 of [SEQ ID NO:2]. The polypeptide preferably comprises all or part of an amino acid sequence in [SEQ ID NO:2]. The invention also includes a polypeptide comprising or consisting of the sequence of SEQ ID NO:2 or 4, or the sequence of SEQ ID NO:2 or 4 with conservative amino acid substitutions. A variation of the invention comprises a polypeptide fragment of the PERK polypeptide, or a peptide mimetic of the PERK polypeptide (eg. PERK1). The polypeptide fragment of the invention preferably comprises at least 20 amino acids, which fragment has PERK activity. The fragment or peptide mimetic is preferably capable of being bound by an antibody to a polypeptide of the invention, such as PERK1. The polypeptide is optionally recombinantly produced. The invention includes an isolated and purified polypeptide comprising the amino acid sequence of a PERK polypeptide, wherein the polypeptide is encoded by a nucleic acid molecule that hybridizes under moderate or stringent conditions to a nucleic acid molecule in [SEQ ID NO:1 or 3], a degenerate form thereof or a complement. The invention also includes a polypeptide comprising an amino acid sequence having greater than 20% sequence identity to the polypeptide of [SEQ ID NO:1 or 3]. The polypeptide preferably comprises a PERK polypeptide. The polypeptide is preferably isolated from Brassica, for example, Brassica napus or Brassica juncea or Brassica rapa or Brassica oleracea.

The polypeptide preferably includes a kinase domain including at least 30% homology to the kinase domain of [SEQ ID NO.:2] and/or an extracellular domain including at least 20% homology to the extracellular domain of [SEQ ID NO.:2]. The invention includes an isolated nucleic acid molecule encoding a polypeptide the invention eg. [SEQ ID NO.:2]. The invention also includes an antibody that binds specifically to a polypeptide of the invention, in particular [SEQ ID NO.:2]. The antibody optionally comprises a monoclonal antibody or a polyclonal antibody. The invention also includes a purified polypeptide that binds specifically to an antibody that binds specifically to PERK1. Such proteins are useful, for example, as a positive control in an assay utilizing the antibody.

The invention also includes an isolated nucleic acid molecule encoding a polypeptide that reduces the severity of wounding or pathogen attack in a plant, the polypeptide comprising:

(a) an extracellular domain which recognizes an extracellular binding molecule whose level is increased during wounding, pathogen attack, or plant growth and development, the extracellular domain encoding a plurality of repeats selected from the group consisting of SPPPP, SPP, PP and PPP, wherein a plurality of the proline molecules are capable being glycosylated and/or hydroxylated;

(b) a membrane domain operably connected to the extracellular domain, wherein the membrane domain is capable of extending across a cell membrane from the extracellular side of the membrane to intracellular side of the membrane; and

(c) a cytoplasmic domain operably connected to the membrane domain, wherein the cytoplasmic domain comprises a means for producing kinase activity when the extracellular binding molecule interacts with the extracellular domain.

The term “isolated nucleic acid” refers to a nucleic acid the structure of which is not identical to that of any naturally occurring nucleic acid or to that of any fragment of a naturally occurring genomic nucleic acid spanning more than three separate genes. The term therefore covers, for example, (a) DNA which has the sequence of part of a naturally occurring genomic DNA molecules; (b) a nucleic acid incorporated into a vector or into the genomic DNA of a prokaryote or eukaryote, respectively, in a manner such that the resulting molecule is not identical to any naturally occurring vector or genomic DNA; (c) a separate molecule such as cDNA, a genomic fragment, a fragment produced by reverse transcription of polyA RNA which can be amplified by PCR, or a restriction fragment; and (c) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

The invention also relates to a method for producing a transgenic plant having increased plant height, number of branches, number of seed pods and/or seed production compared to a non-transgenic plant, and/or quicker flowering or later senescence compared to a non-transgenic plant, comprising transforming a plant with a vector including a PERK nucleic acid molecule or a nucleic acid molecule having PERK activity.

Another aspect of the invention relates to a method of producing a genetically transformed plant which expresses a PERK polypeptide or a polypeptide having PERK activity, the plant having increased plant height, number of branches, number of seed pods and/or seed production, compared to wild-type plants, and/or quicker flowering or later senescence compared to a non-transgenic plant, comprising:

(a) cloning or synthesizing a PERK nucleic acid molecule or nucleic acid molecule having PERK activity;

(b) inserting the nucleic acid molecule in a vector so that the nucleic acid molecule is operably linked to a promoter;

(c) inserting the vector into a plant cell or plant seed;

(d) regenerating the plant from the plant cell or plant seed, wherein plant height, number of branches, number of seed pods and/or seed production compared to wild-type plants in the plant are increased or wherein the plant has quicker flowering or later senescence.

The invention also relates to the foregoing methods wherein the isolated nucleic acid molecule encoding a PERK polypeptide or polypeptide having PERK activity comprises a nucleic acid molecule selected from the group consisting of:

a) a nucleic acid molecule that hybridizes to a nucleic acid molecule consisting of all or part of [SEQ ID NO:1], or a complement thereof under low, moderate or high stringency hybridization conditions;

b) a nucleic acid molecule degenerate with respect to (a), wherein the nucleic molecule encodes a PERK polypeptide or a polypeptide having PERK activity.

In the foregoing methods, the nucleic acid molecule encoding a PERK polypeptide, or a polypeptide having PERK activity, optionally comprises a nucleic acid molecule selected from the group consisting of:

(a) the nucleic acid molecule of the coding strand shown in [SEQ ID NO:1], or a complement thereof;

(b) a nucleic acid molecule encoding the same amino acid sequence as a nucleotide sequence of (a); and

(c) a nucleic acid molecule having at least 17% identity with the nucleotide sequence of (a) and which encodes a PERK polypeptide or a poypeptide having PERK activity.

The plant used in the methods may be of a species selected from the group consisting of alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rape, rice, roses, rye, salicornia sorghum, soybean, spinach, squash, strawberries, sunflower, sweet corn, tobacco, tomato and wheat.

### BRIEF DESCRIPTION OF THE DRAWINGS

Preferred embodiments will be described in relation to the drawings in which:

FIG. 1. Nucleotide and Deduced Amino Sequence. [SEQ ID NOS.: 1 & 2]

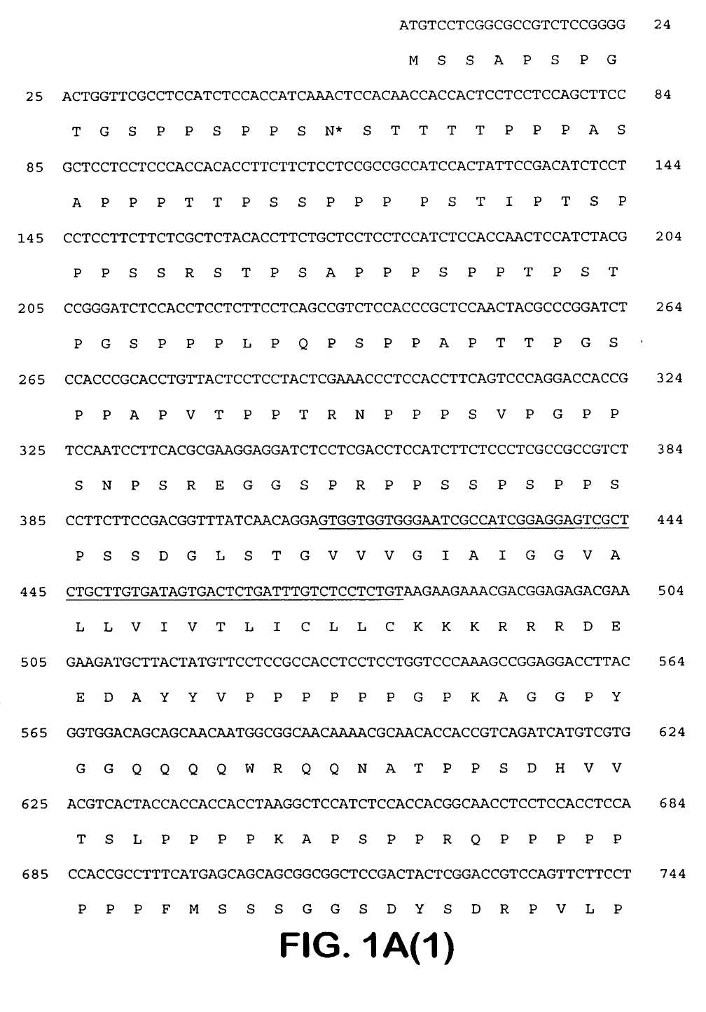

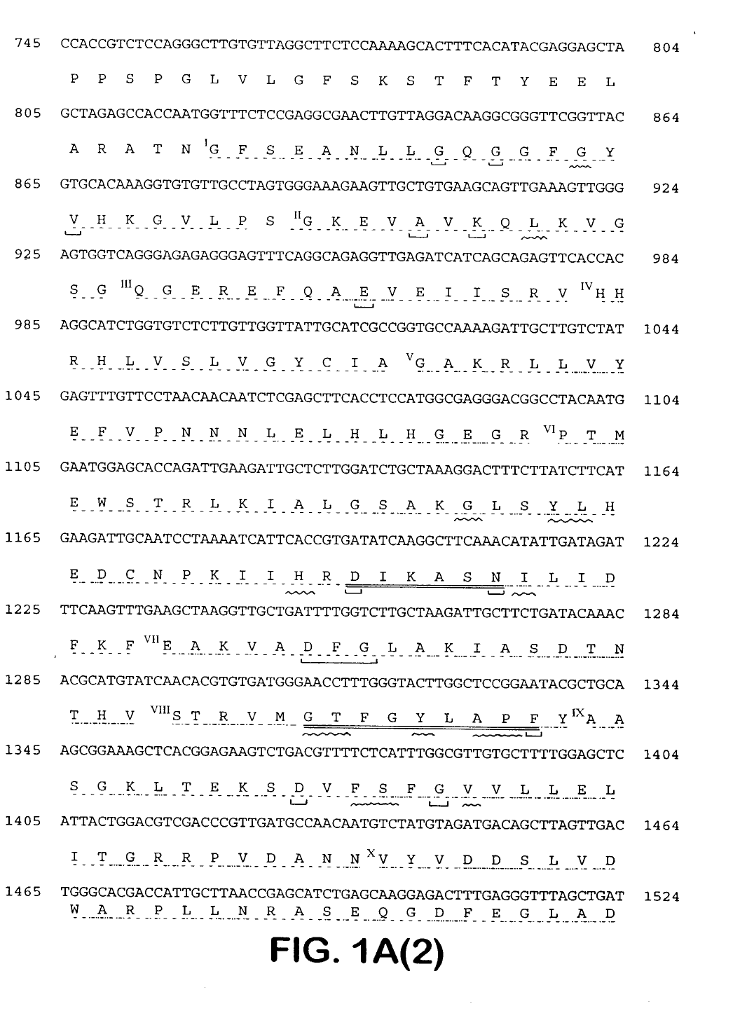

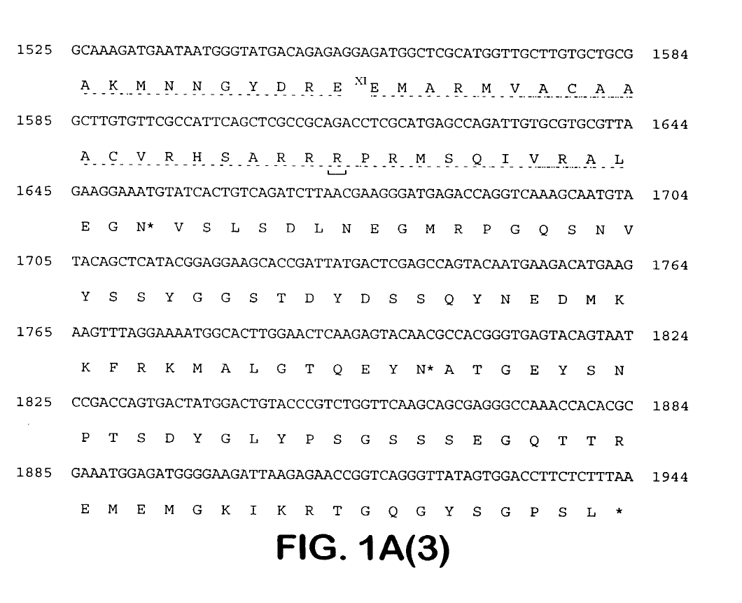

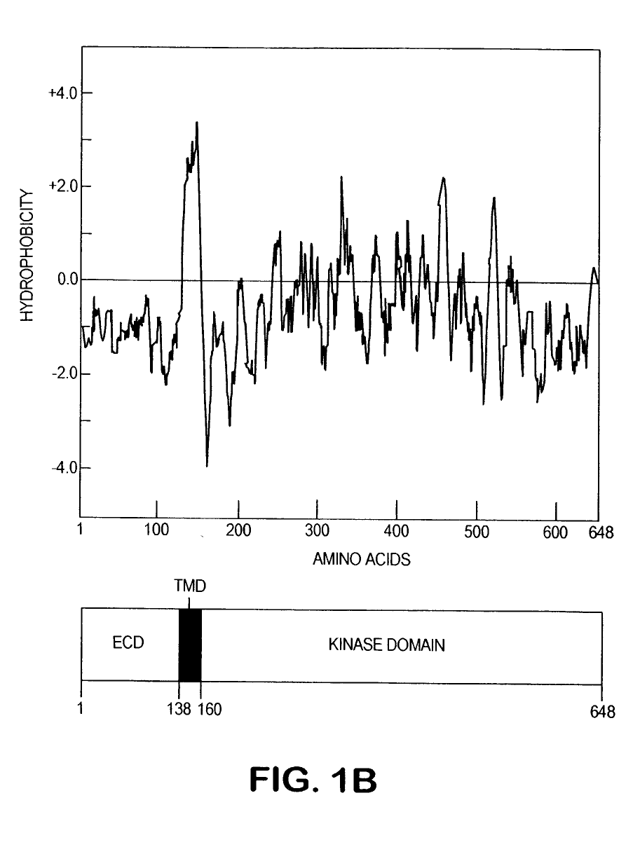

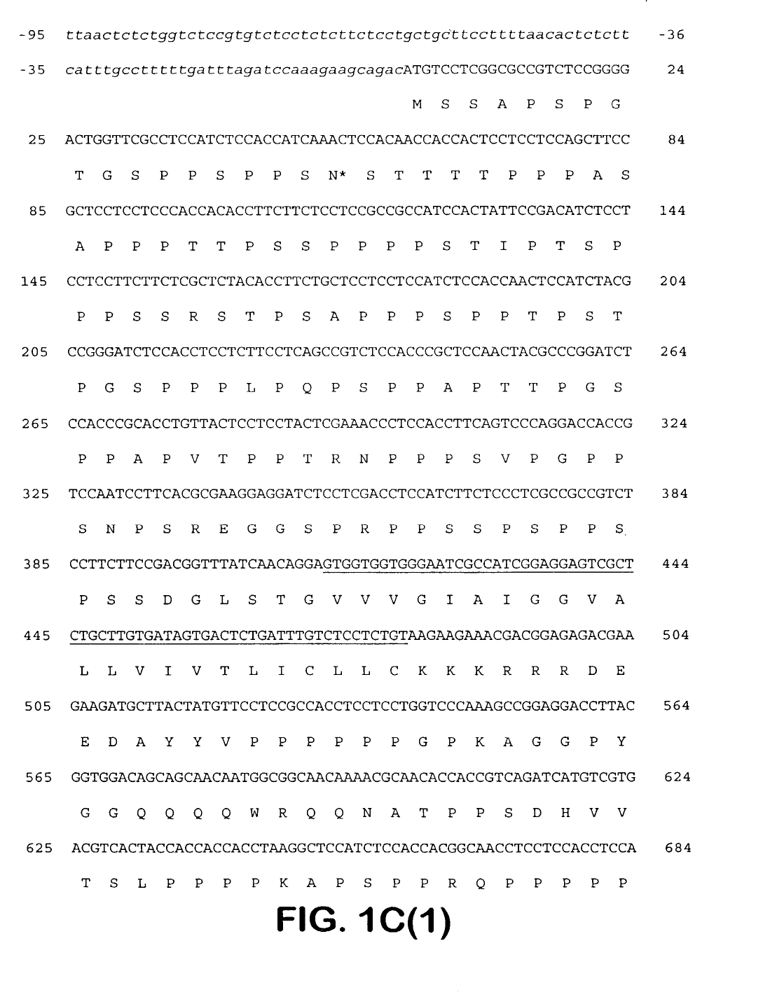

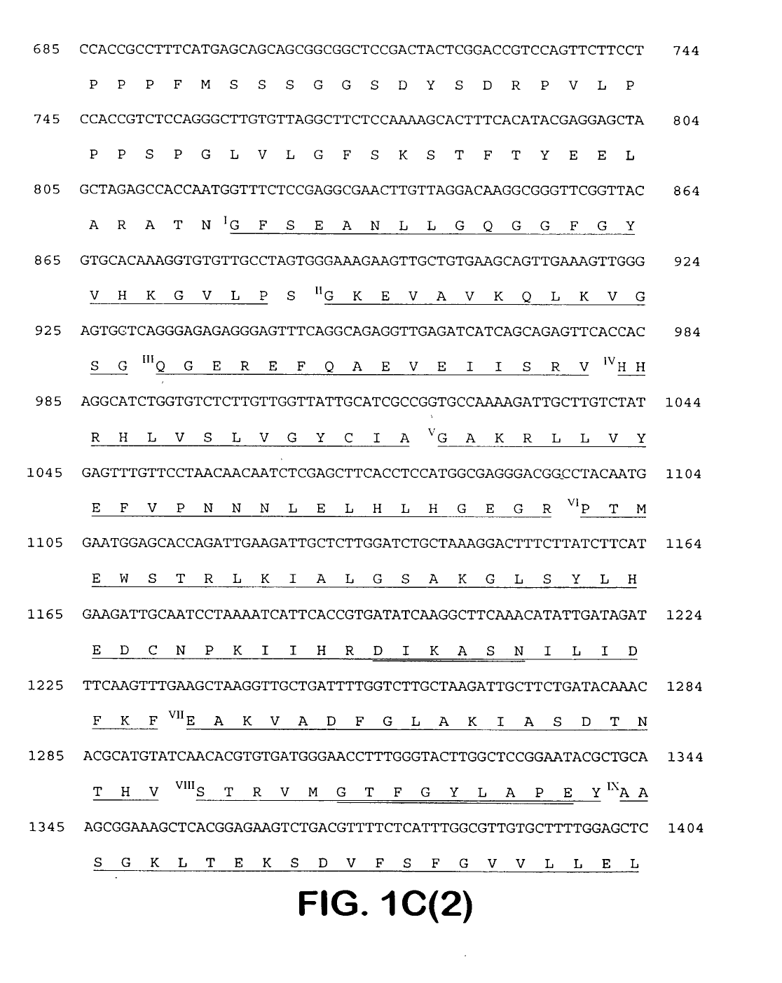

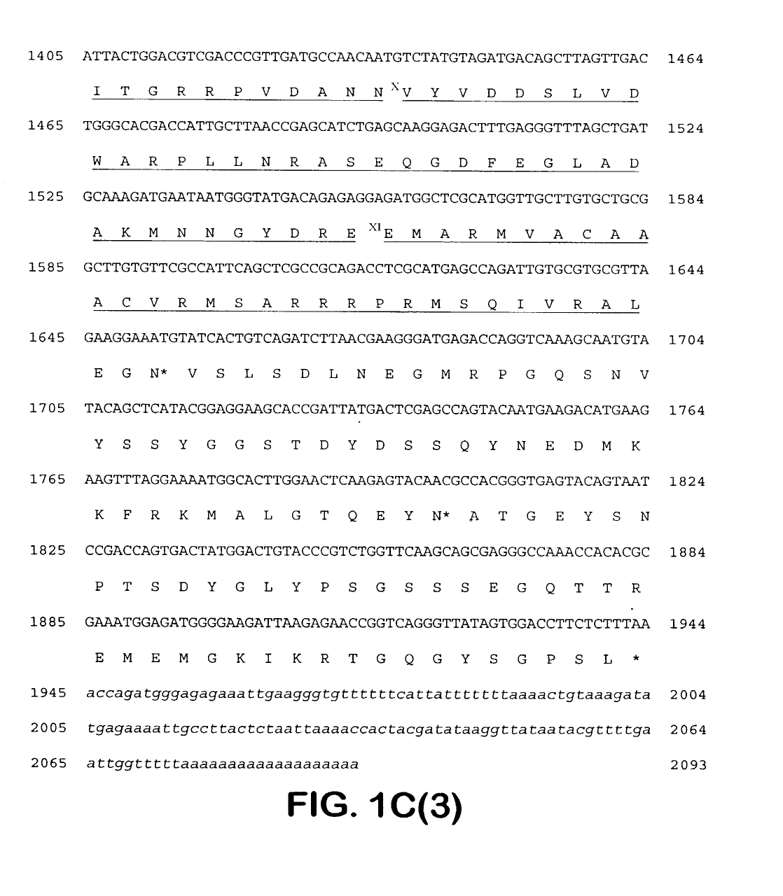

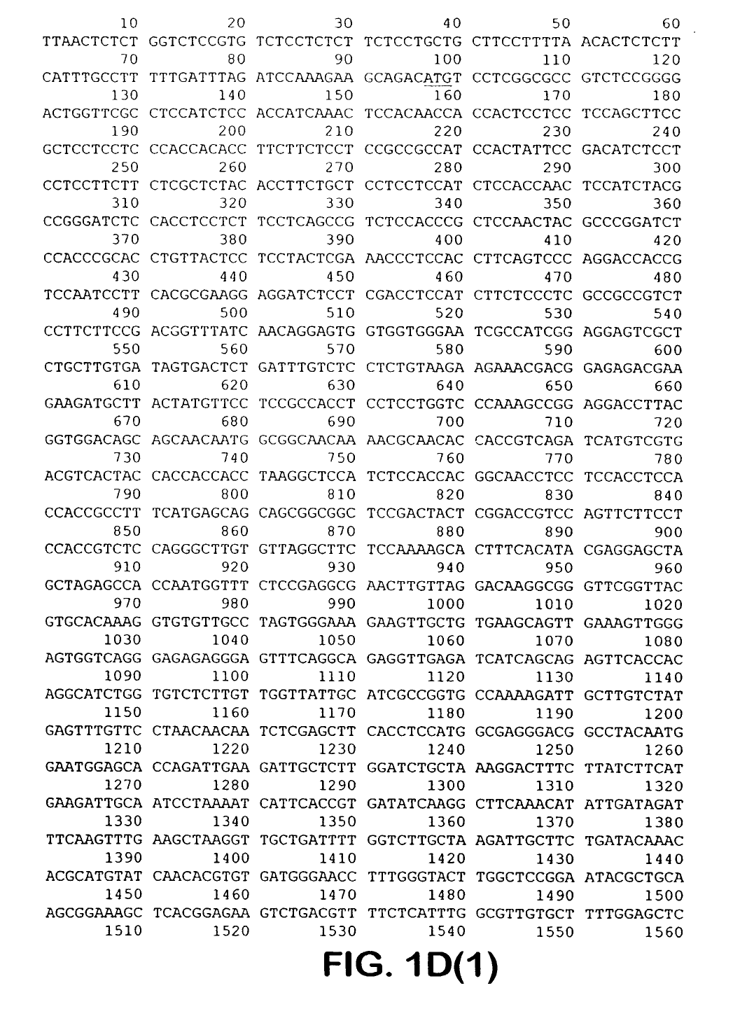

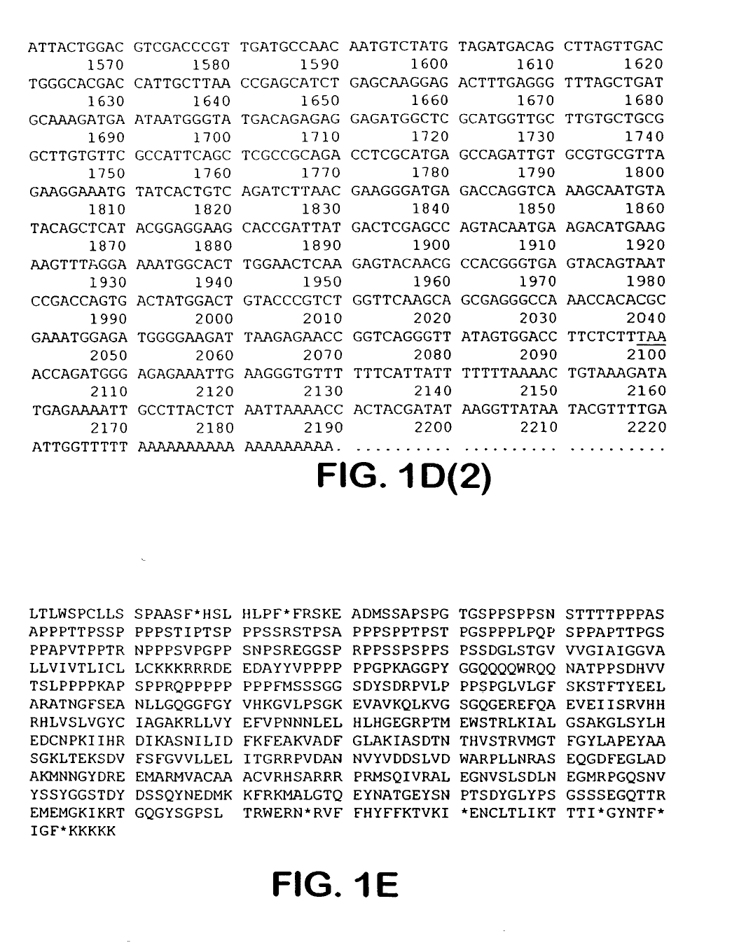

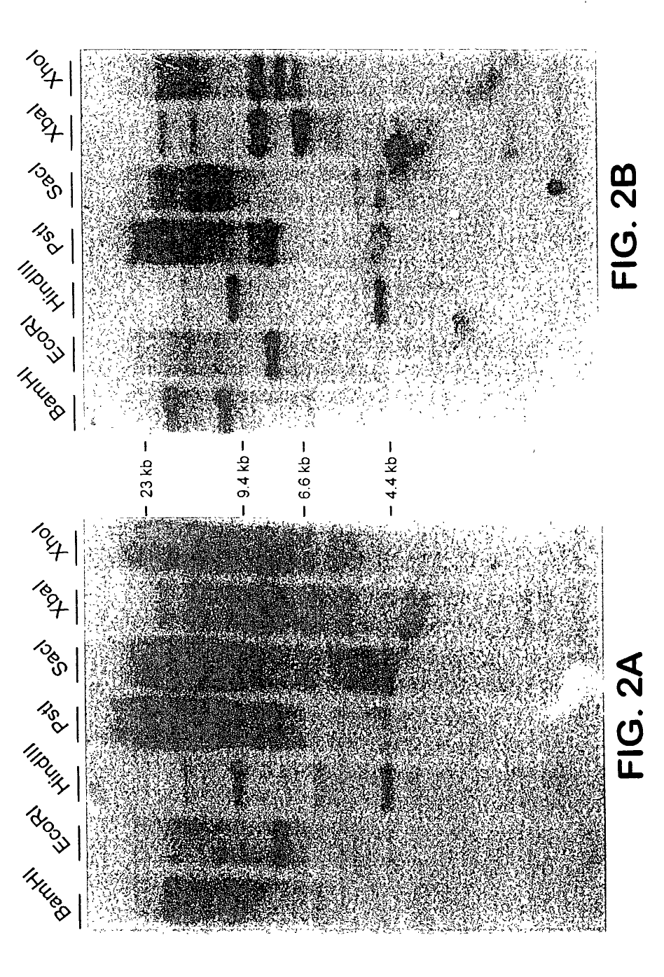

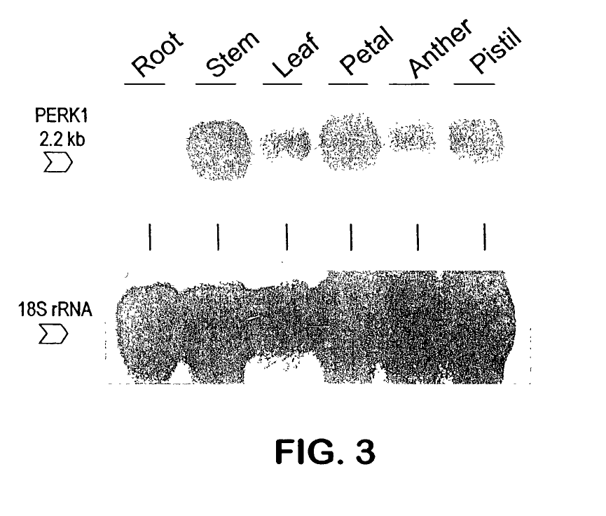

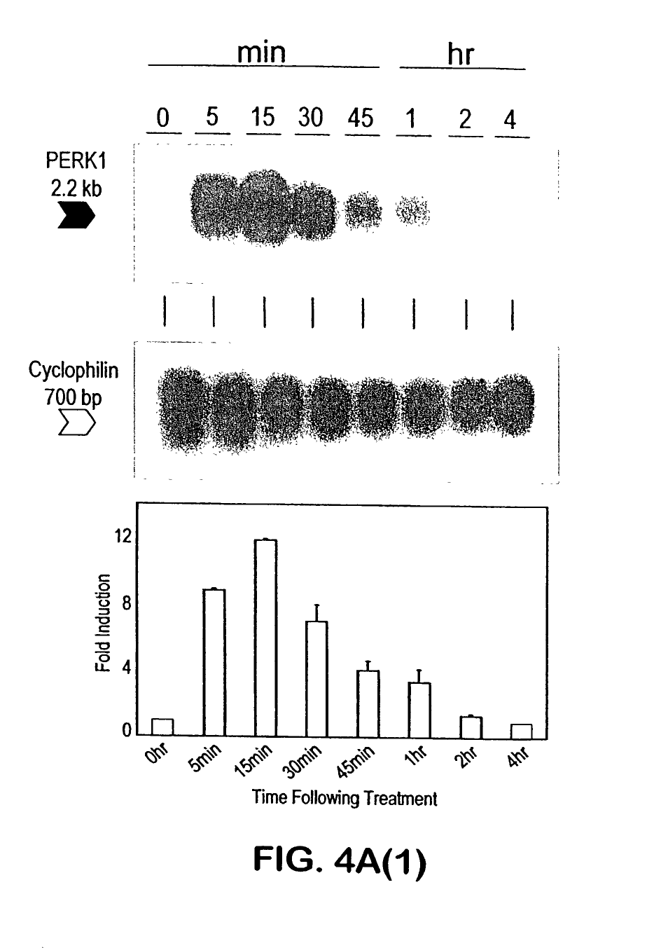

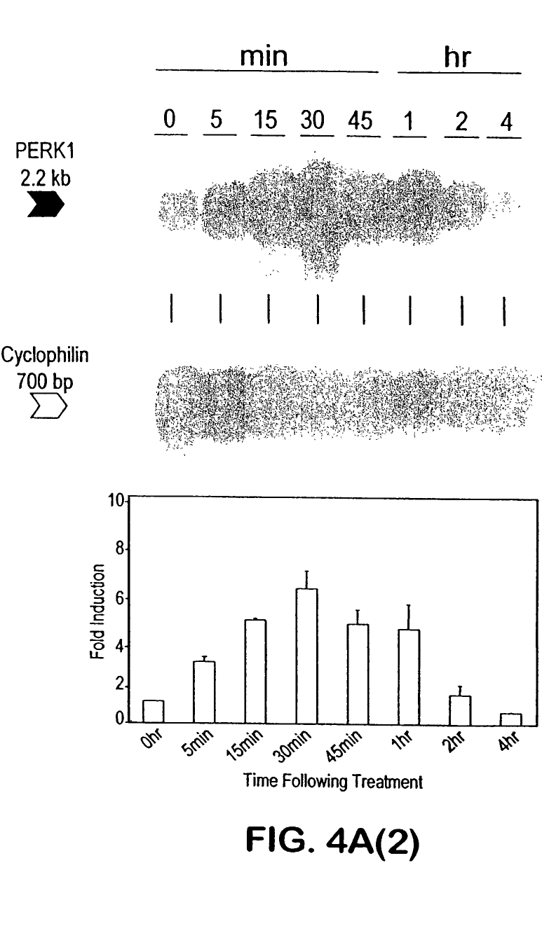

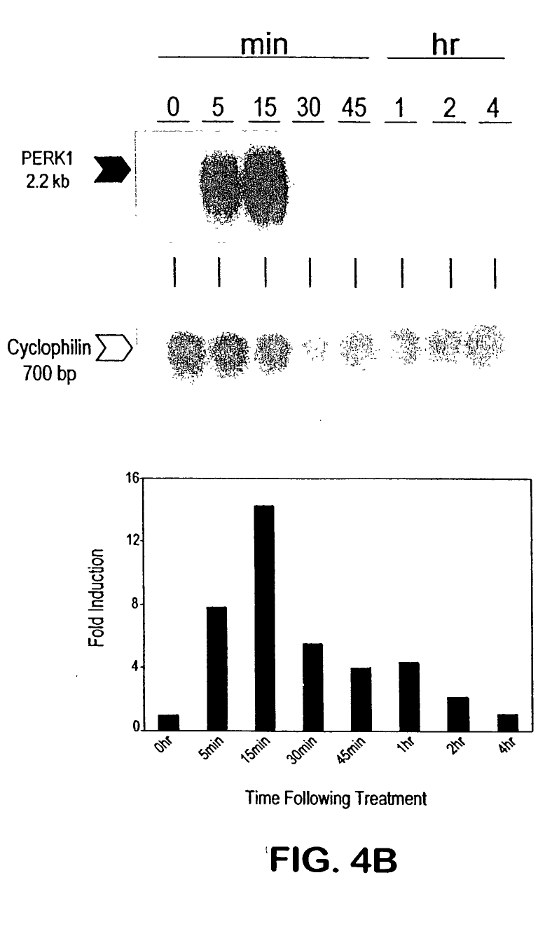

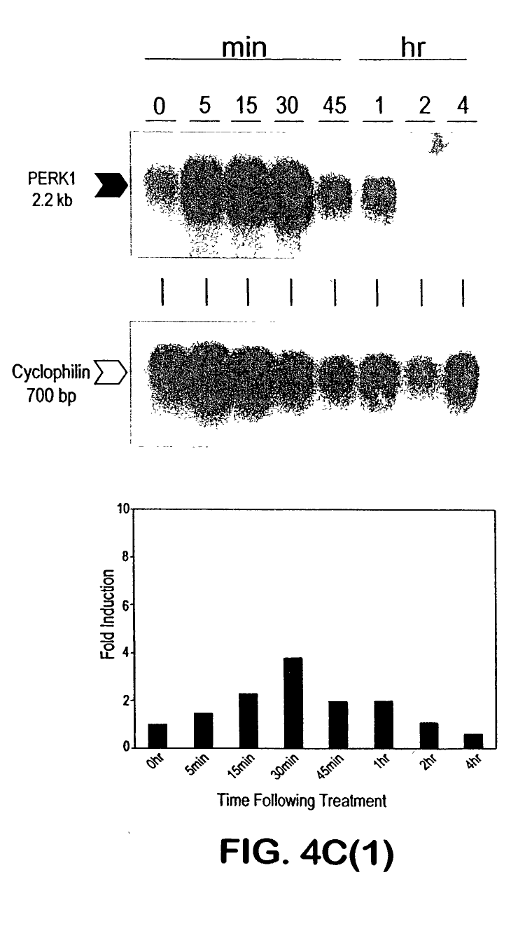

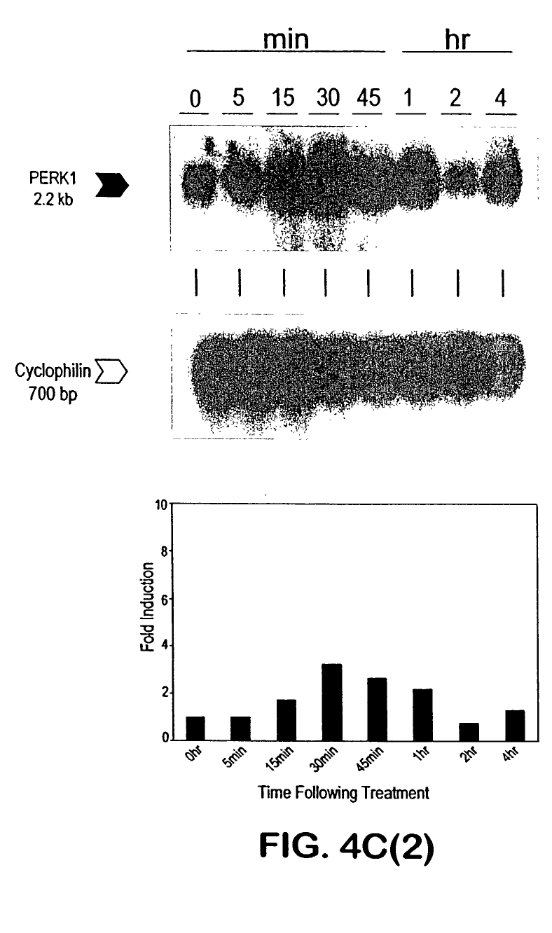

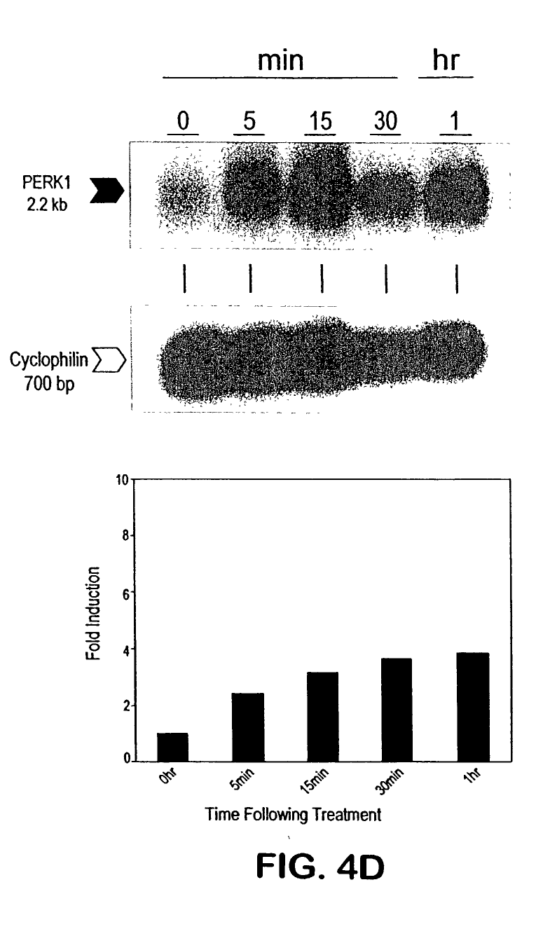

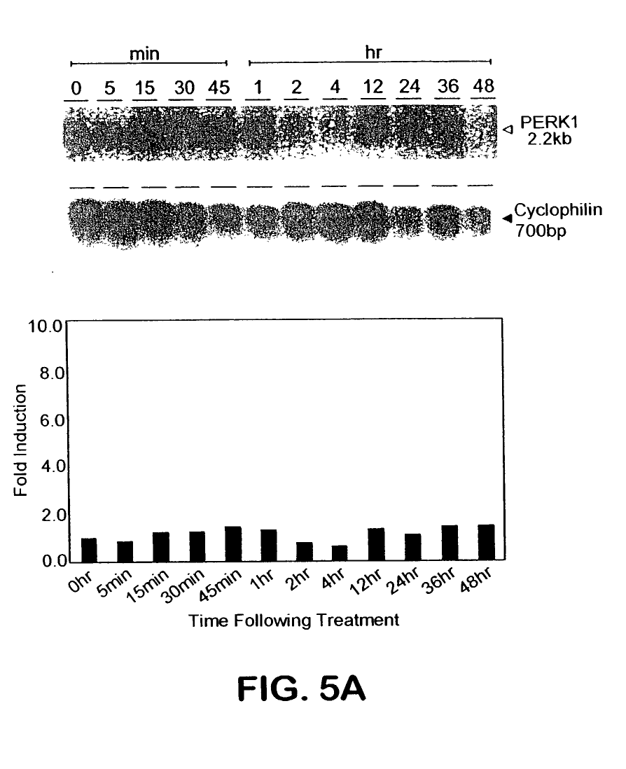

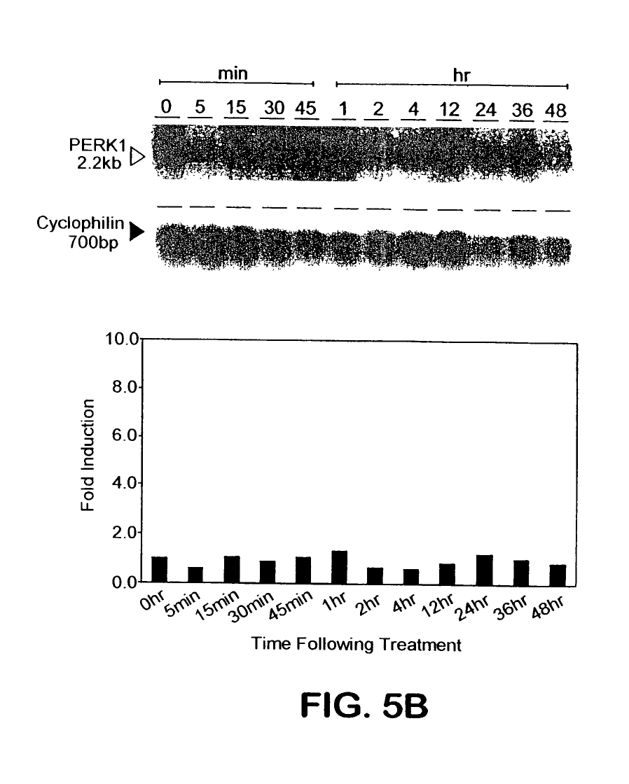

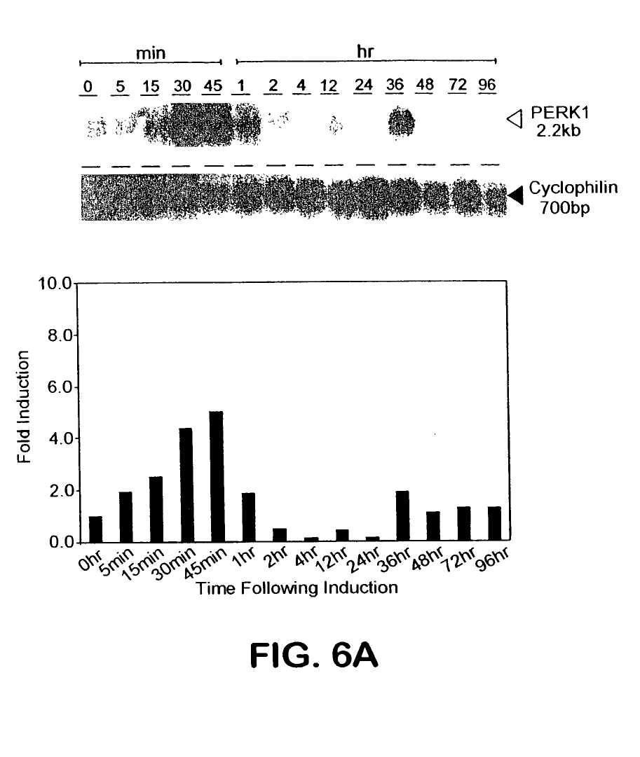

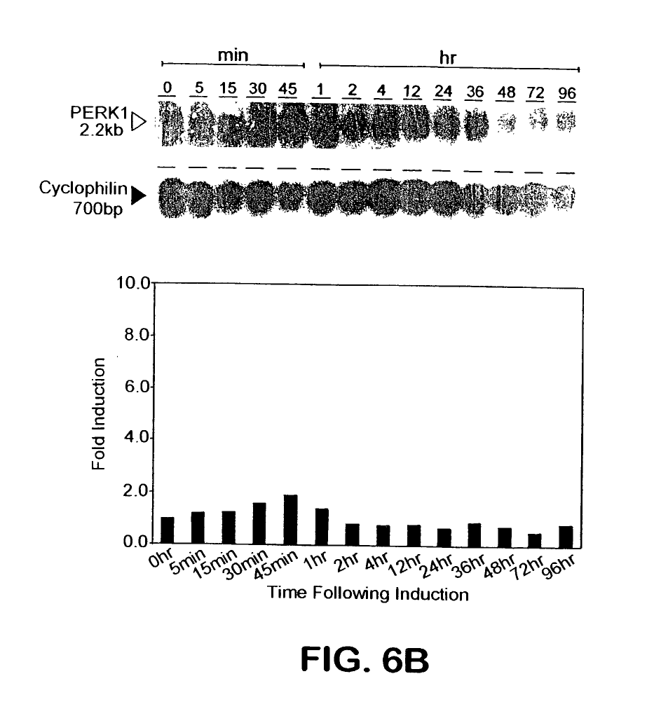

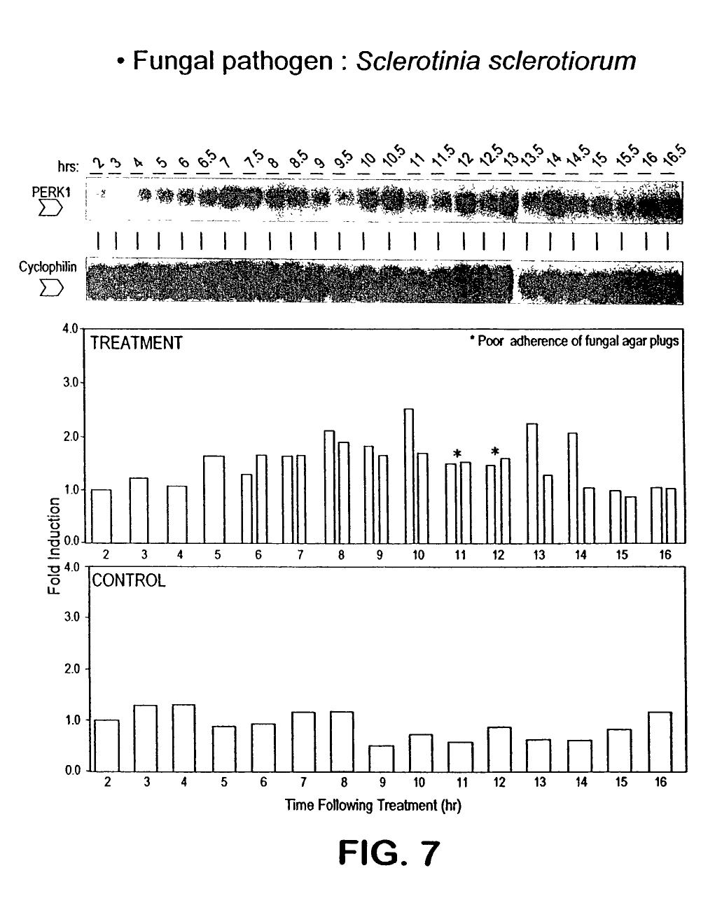

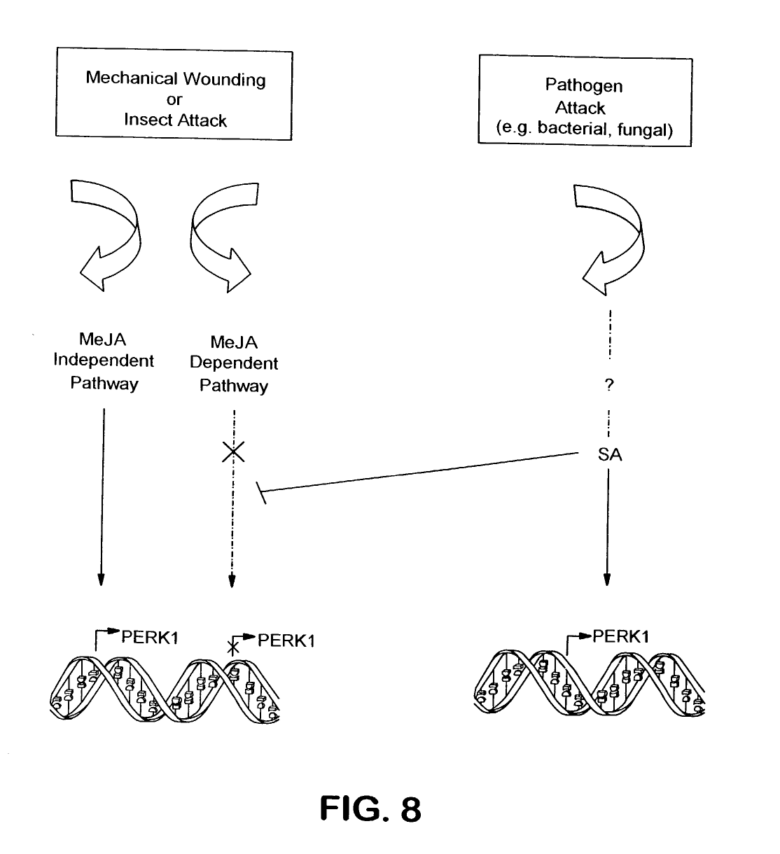

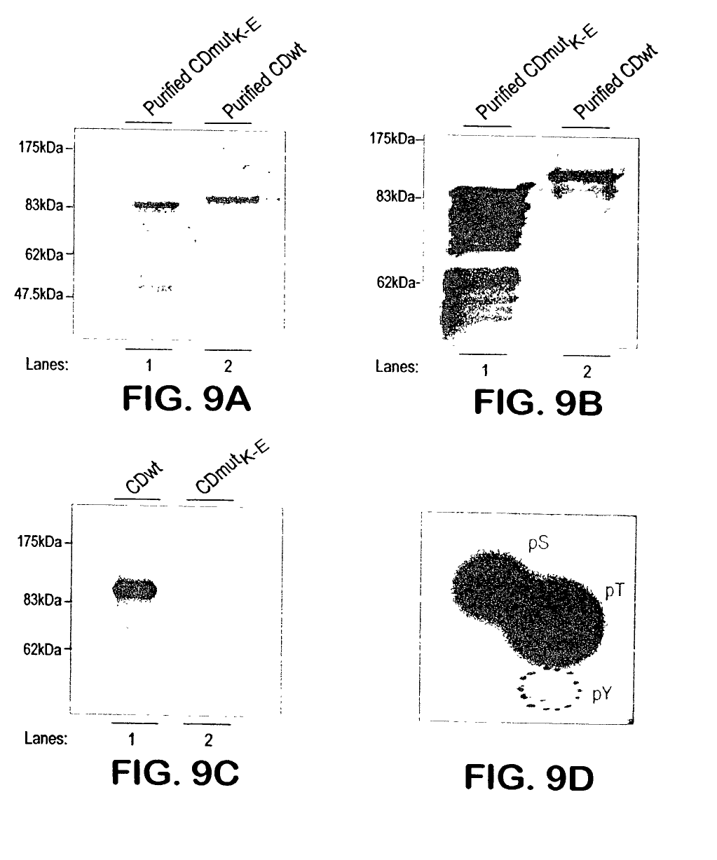

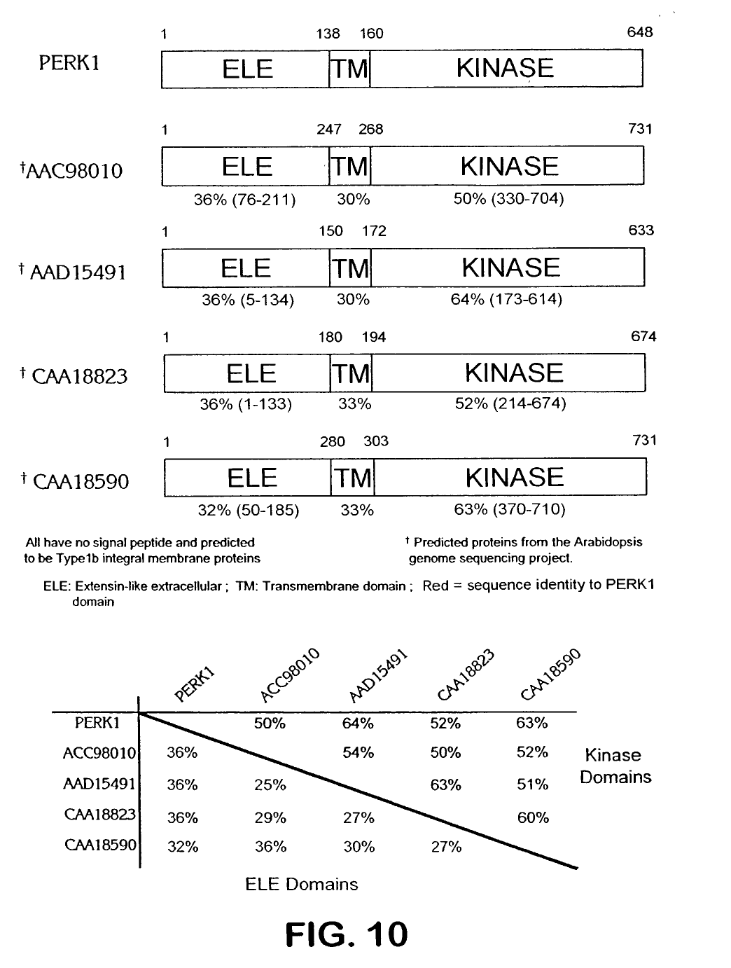

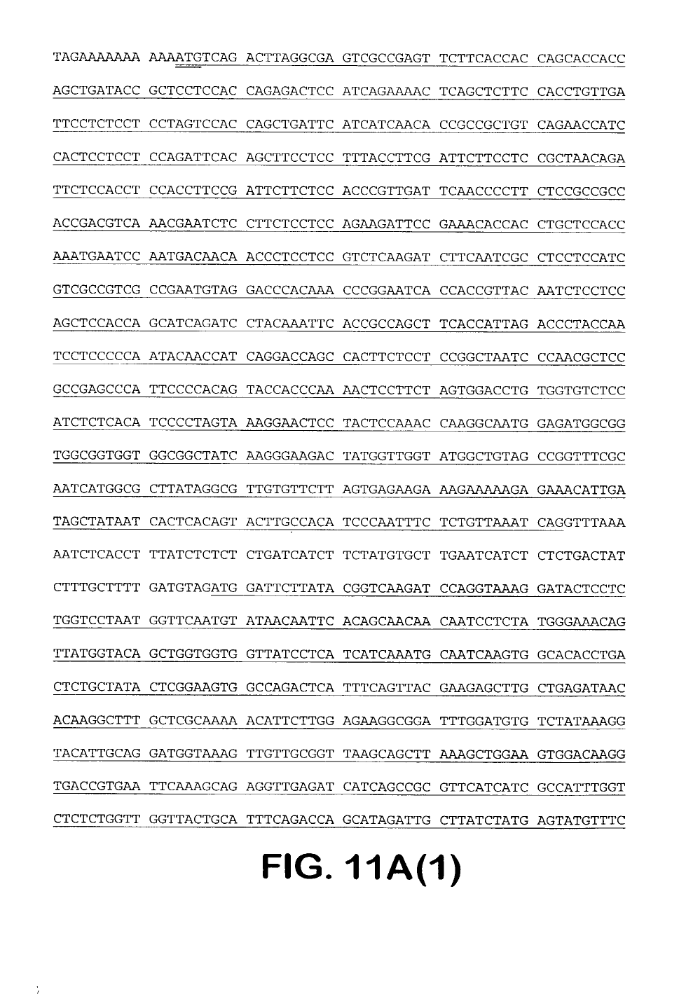

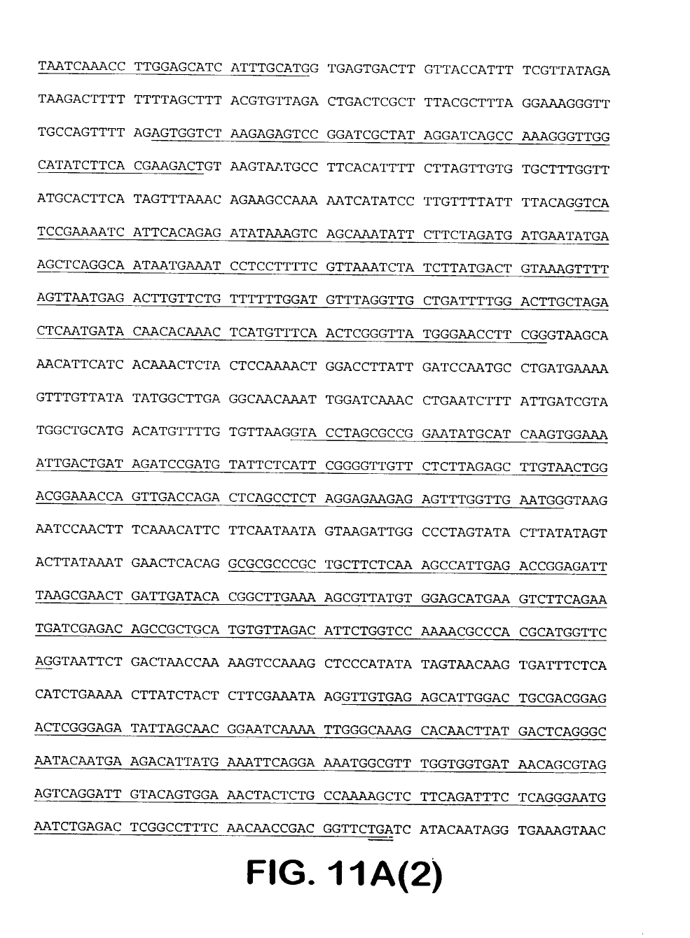

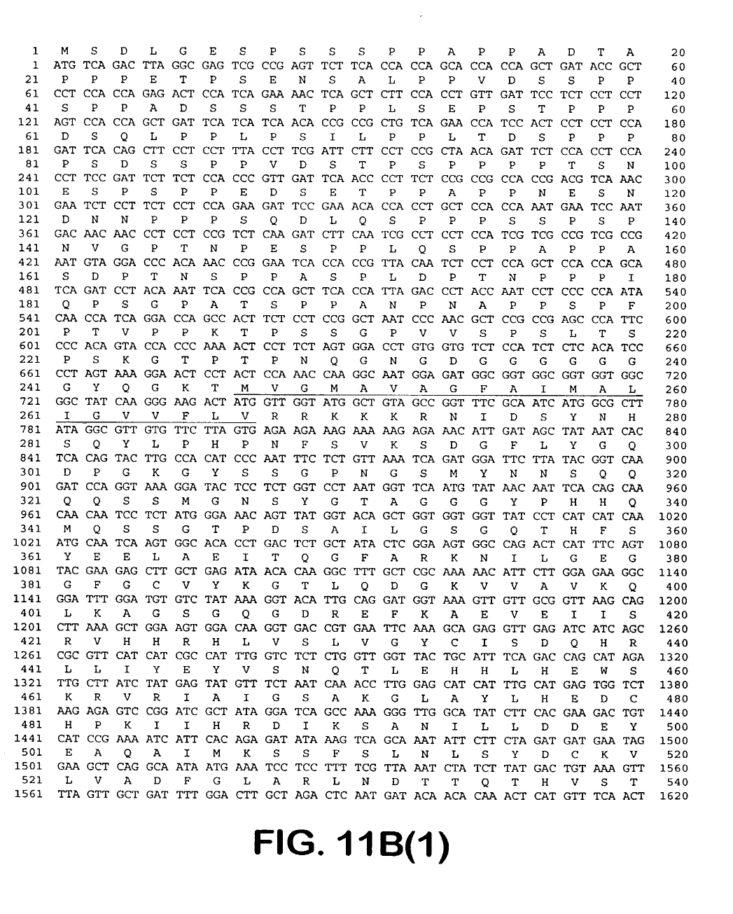

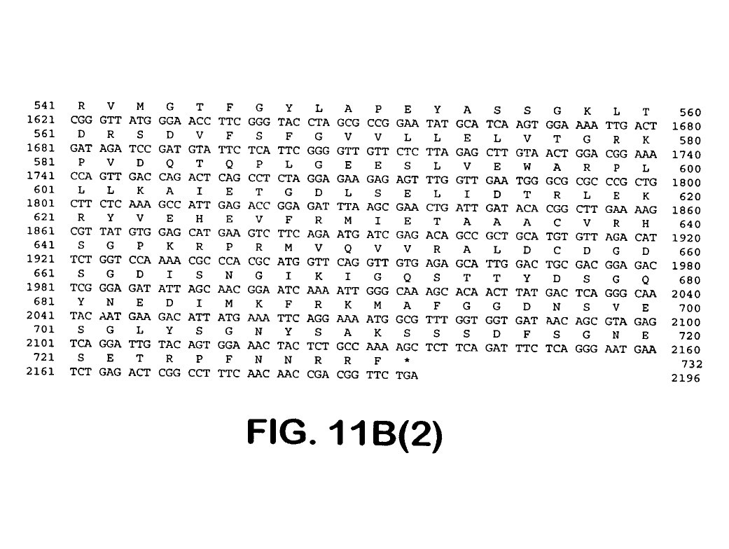

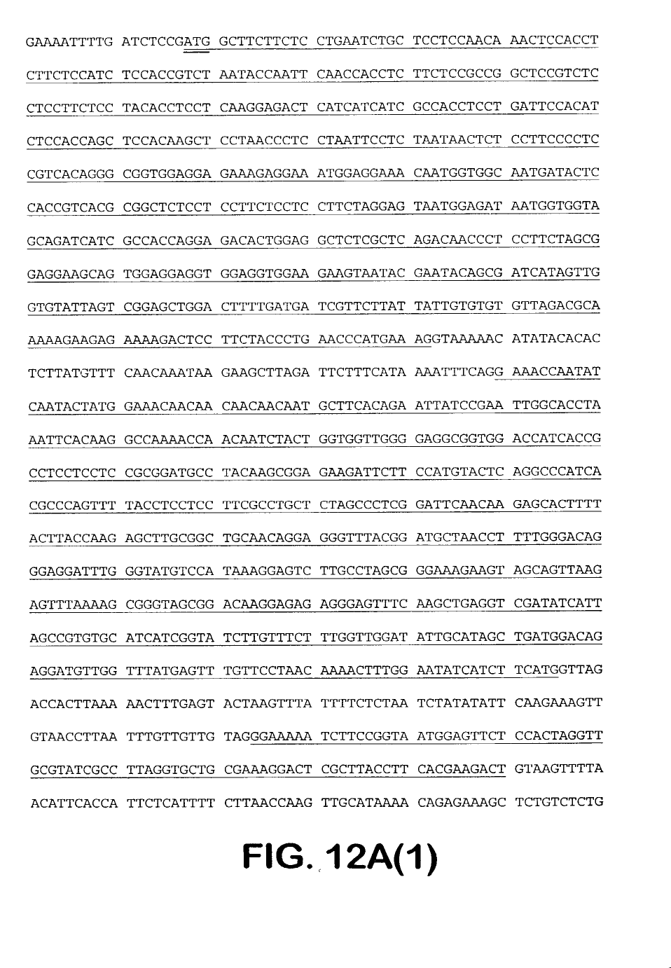

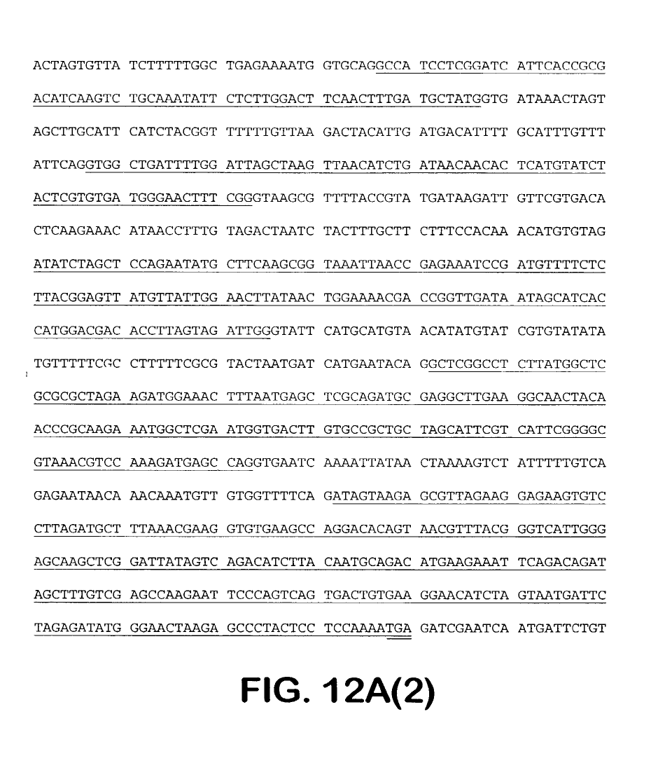

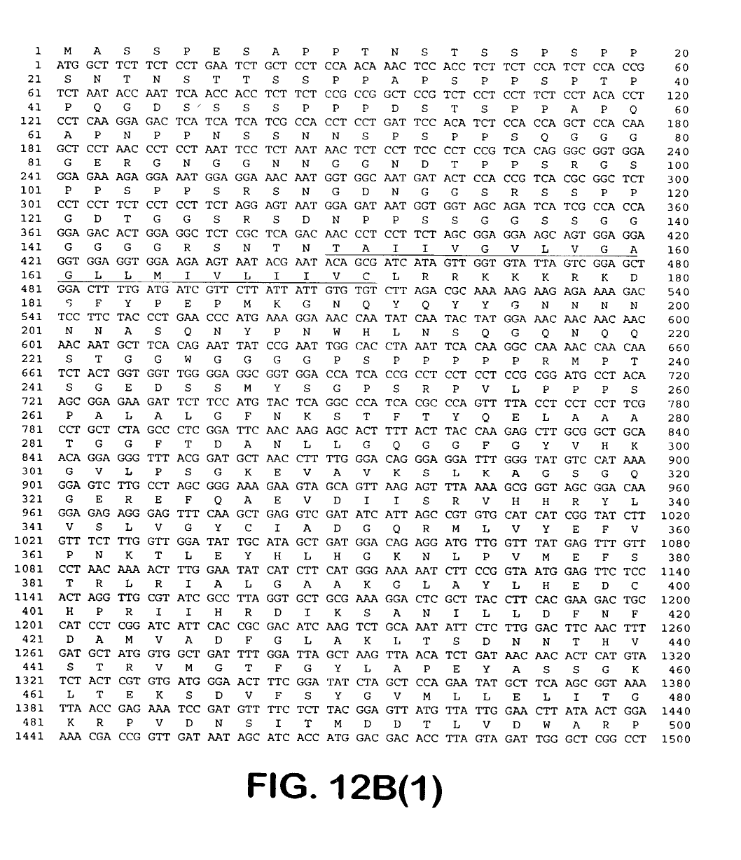

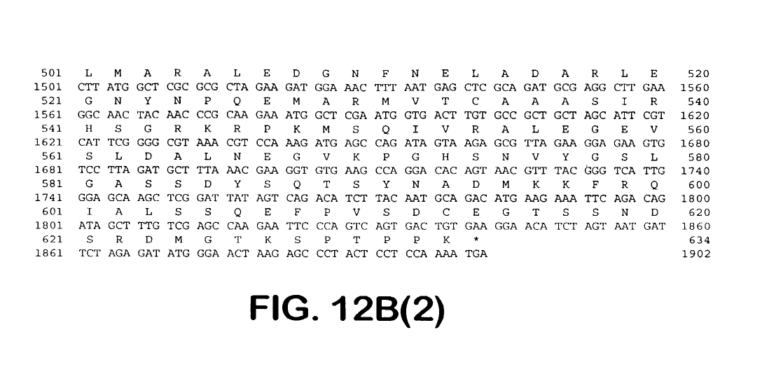

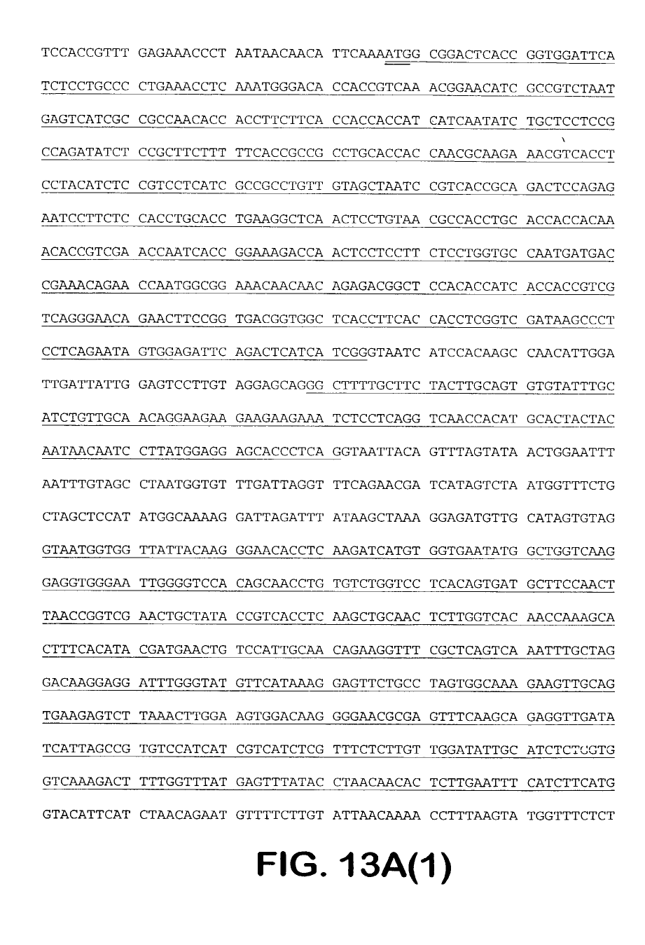

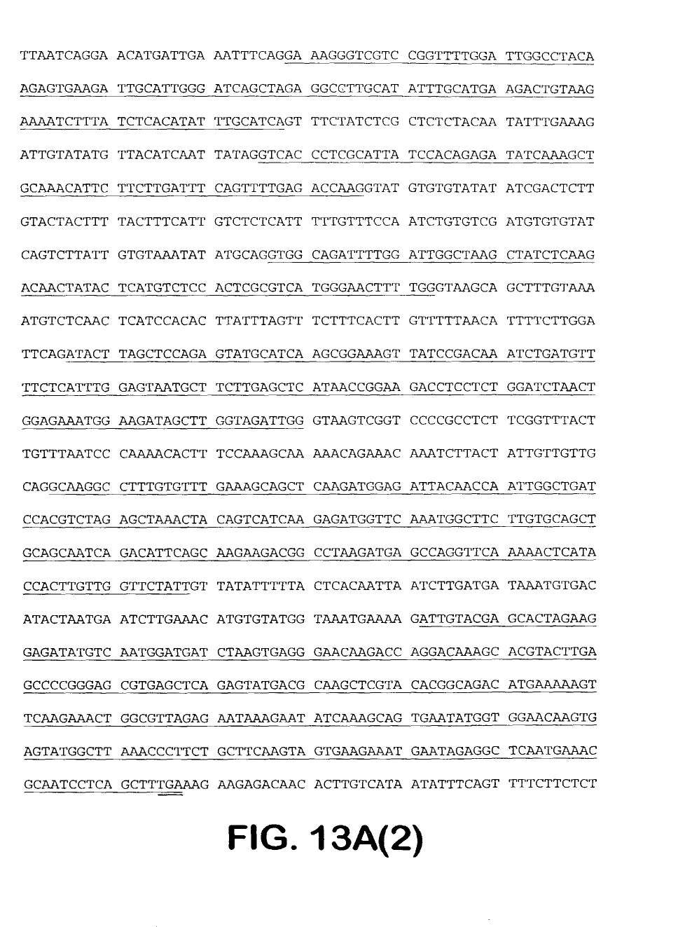

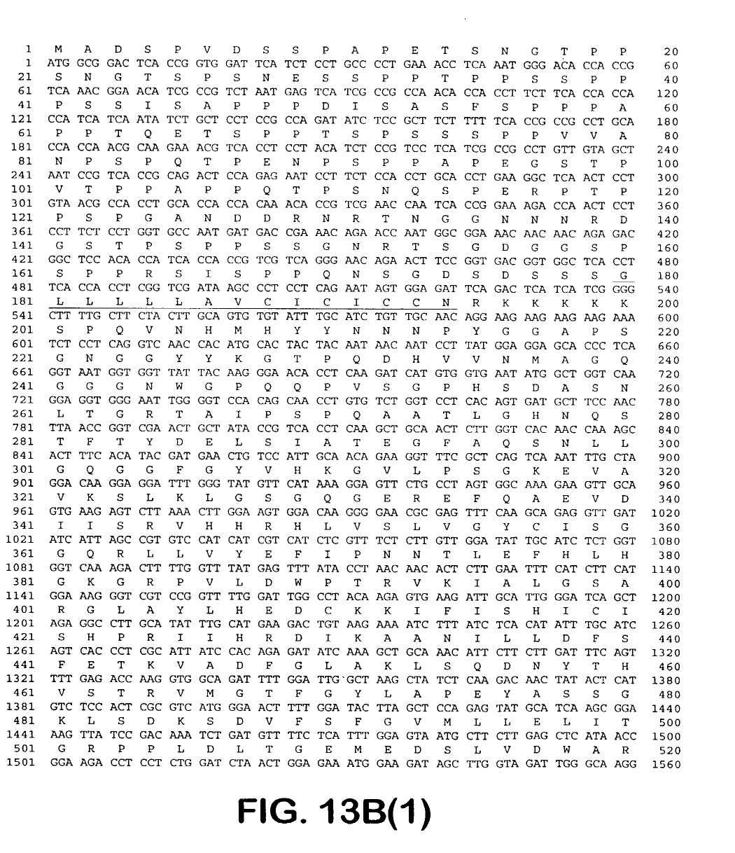

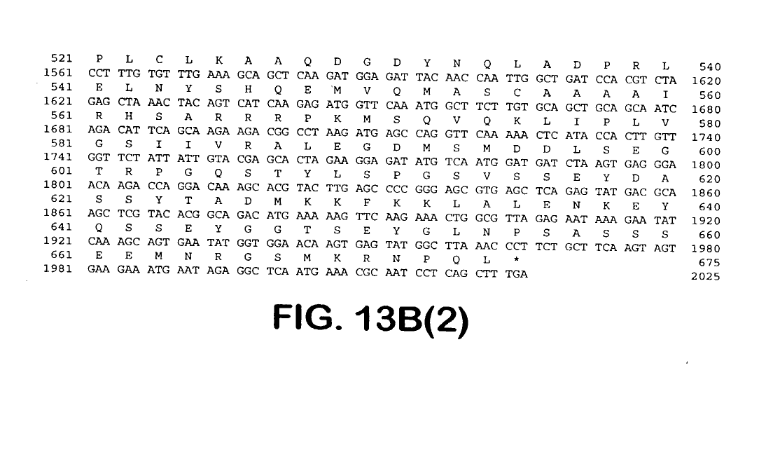

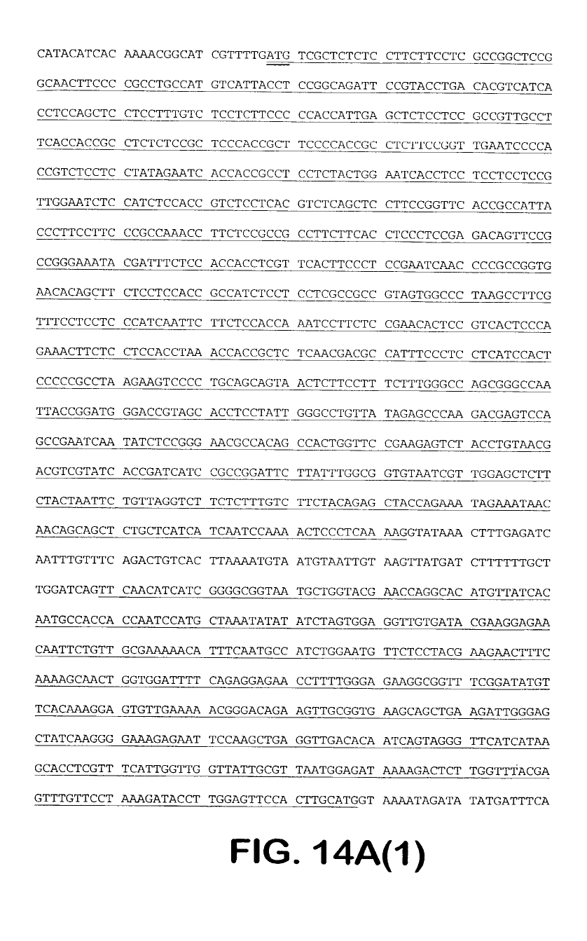

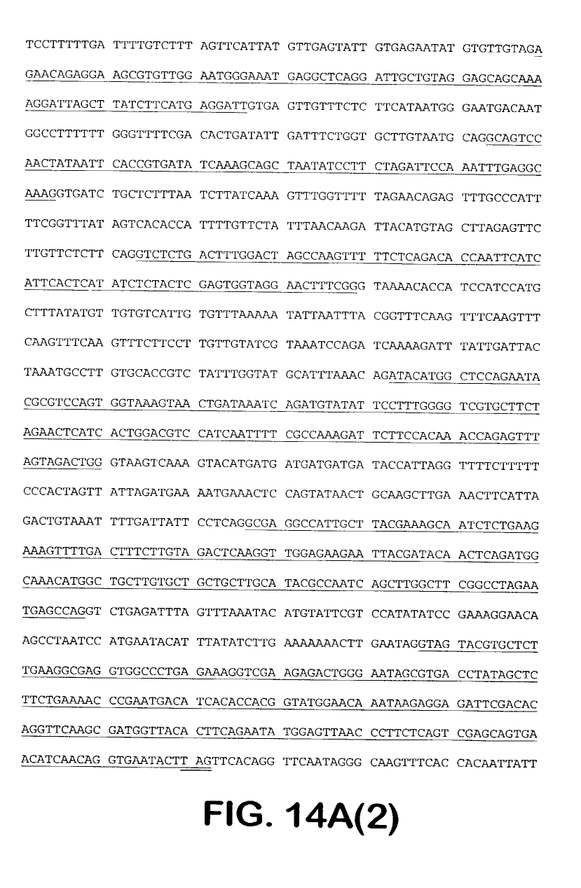

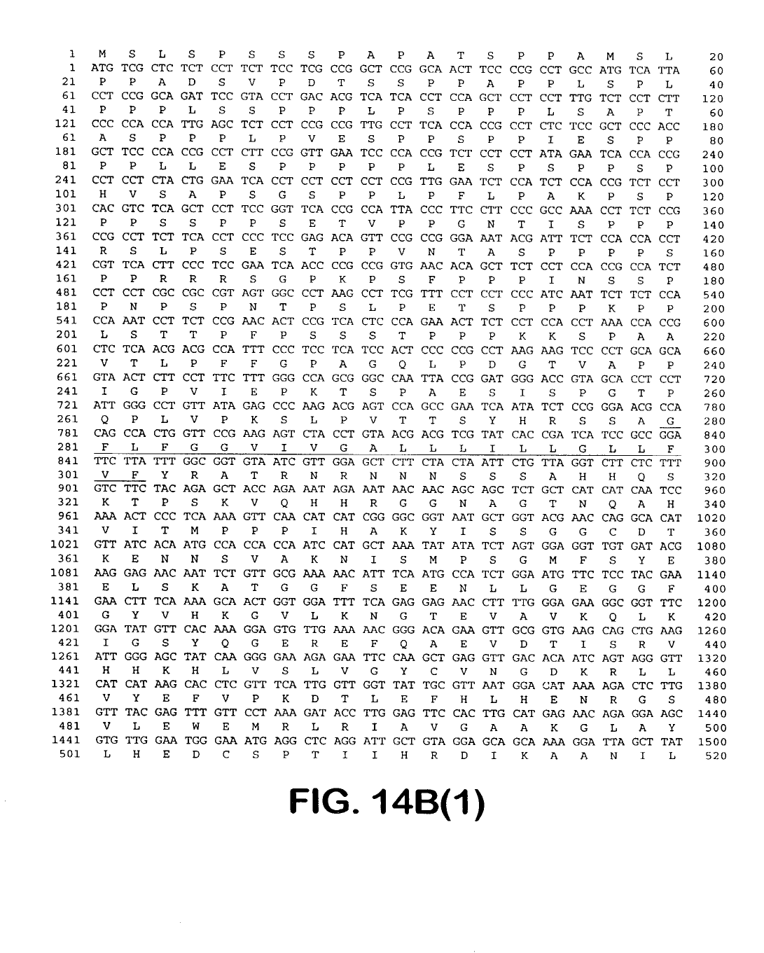

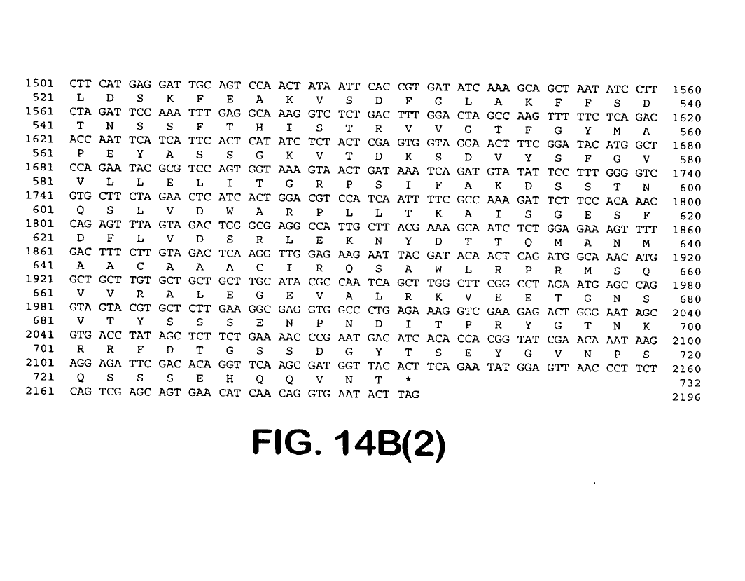

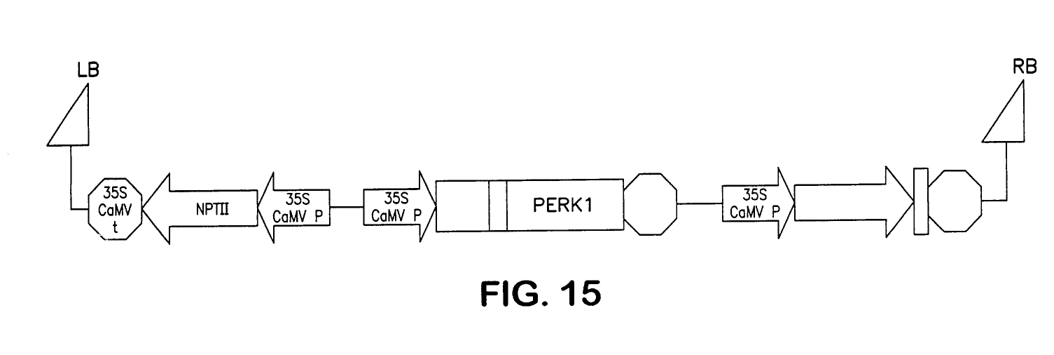

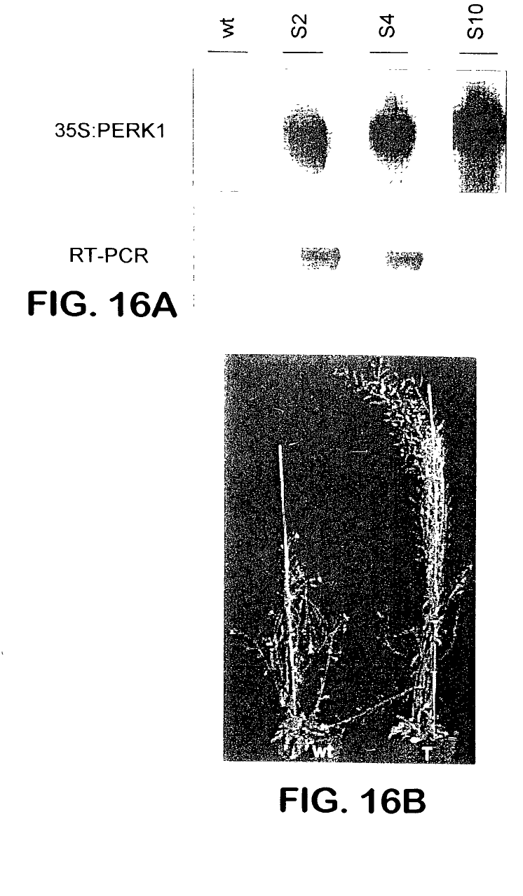

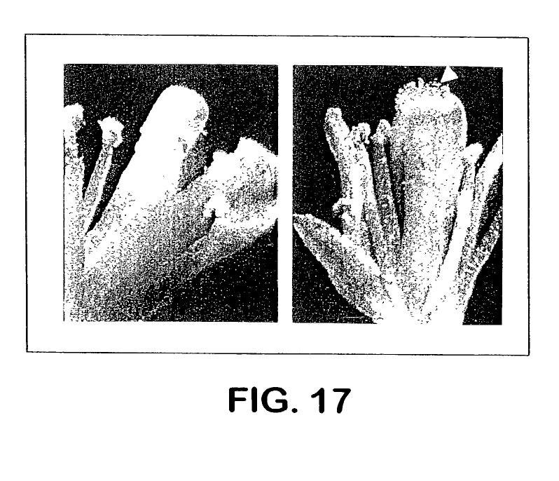

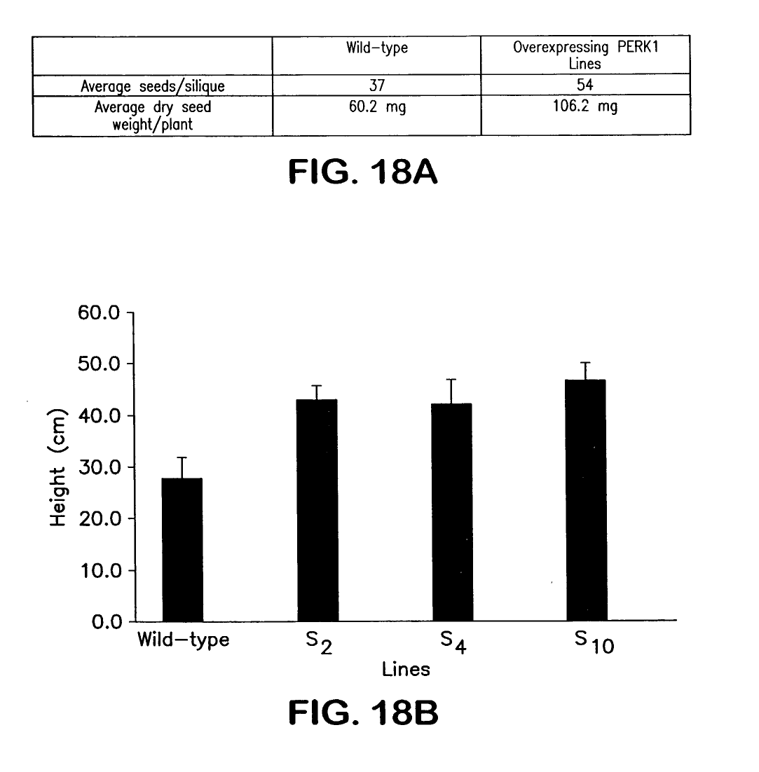

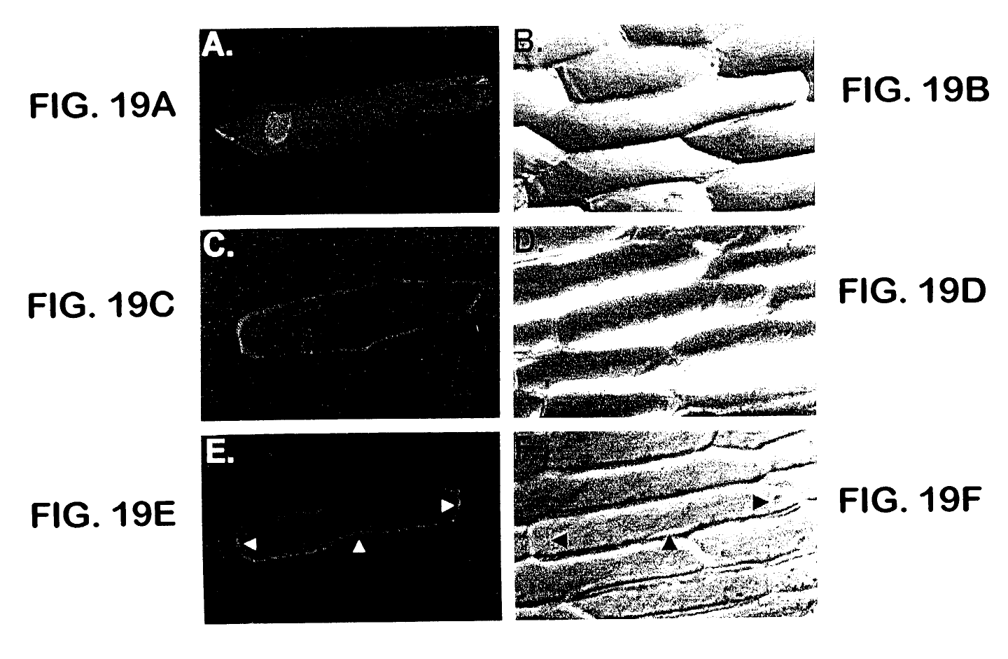

(A) FIG. 1 shows the nucleic acid molecule of [SEQ ID NO.: 1] and the amino acid sequence of [SEQ ID NO.: 2].

In a preferred embodiment, the figure shows the nucleotide sequence corresponding to the PERK1 cDNA, with the predicted amino acid sequence presented as a single-letter code below the nucleotide sequence. Numbers to the left refer to nucleotide sequence and the 5′ and 3′ untranslated regions are presented in lower case letters. Potential N-glycosylation site (Asn-x-Ser/Thre) are indicated by dots above the Asn residues, and the extensin signature (Ser-Pro)₄ pentapeptide motif present in the extracellular domain is denoted in boldface type. The predicted membrane spanning region is marked by a solid underline. As defined by Hanks and Quinn (1991), the catalytic domain has been subdivided into 11 subdomains marked by dashed underlines and superscript roman numerals. The amino acids with a bracket underneath represent residues that are absolutely conserved, whereas amino acids with a wavy line underneath represent groups of conserved amino acids. The two regions marked by double underlines represent consensus sequences common amongst serine/threonine kinases.

(B) Structural features of the PERK1 polypeptide. A Kyte hydropathy plot (Kyte and Doolittle, 1982) of the amino acid sequence generated by DNAsis® software (Hitachi Software, San Bruno, Calif.) is shown, where increased hydrophobicity is denoted by positive values. The domains of the PERK1 protein are illustrated below. ECD, extracellular domain; TM, transmembrane domain.

(C) Shows the nucleic acid molecule of [SEQ ID NO.: 3] and the amino acid sequence of [SEQ ID NO.: 4]. These sequences are identical to nucleotides 1 to 1944 of [SEQ ID NO:1] and the corresponding amino acid sequence but they also include 5′ and 3′ untranslated nucleotide regions. (D) Shows the nucleic acid molecule of [SEQ ID NO.: 3] including the 5′ and 3′ untranslated regions. The start and stop codons are underlined and in light print. The nucleotide numbers correspond to the entire sequence and not only to the coding region. In a preferred embodiment, this is the coding sequence of PERK1.

(E) FIG. 1 shows the amino acid sequence of [SEQ ID NO.: 5] including amino acids corresponding to the 5′ and 3′ untranslated regions. The starting methionine is in light print and the stop codon is indicated by an asterisk. It will be clear to those skilled in the art that one may only use the portion of the amino acid sequence between the start and stop codons.

FIG. 2. Genomic DNA Southern Blot Analysis of PERK1.

Genomic DNA (5 micrograms) isolated from Brassica napus leaf tissue was digested with the indicated restriction enzymes, blotted and hybridized with a partial 1.5 kb PERK1 cDNA probe under varying conditions of stringency. DNA markers are indicated in kilobases.

(A) Genomic DNA gel blot analysis under low stringency conditions.

(B) Genomic DNA gel blot analysis under high stringency conditions.

FIG. 3. Expression of PERK1 cDNA

(A) RNA gel blot analysis of PERK1 transcripts from total RNA extracted from various Brassica napus tissues. The blot was hybridized with the full length PERK1 cDNA probe and the expected ˜2.2 kb PERK1 transcript was detected (upper panel).

(B) The blot was subsequently probed with 18S rRNA as an internal control for even loading (lower panel).

FIG. 4. Wound-Inducible Accumulation of PERK1 mRNA in Brassica napus Leaf and Stem Tissue.

(A) Fully expanded leaves were wounded by punching out discs around the perimeter of the leaf blade. Wounds mimic injury inflicted on plants in the field as a result of insect attack or other mechanical damage. Total RNA was extracted at various time intervals after treatment, subjected to Northern blot analysis and probed with full length PERK1 cDNA (bold-face arrow). The blot was reprobed with cyclophilin used as an internal control for even loading (open-face arrow). The graph represents the expression profile of PERK1 in response to wounding corrected against levels of cyclophilin expression. Error bars represent the standard error derived from two independent experiments. Control unwounded leaf tissue represented by 0 hr time point.

(B) Northern blot showing a time-course induction of PERK1 mRNA accumulation in wounded stem tissue. Total RNA harvested at the indicated time points was blotted and hybridized against the full length PERK1 coding sequence (bold-face arrow). The cyclophilin loading control (open-face arrow) was used to normalize levels of PERK1 mRNA accumulation represented graphically. Error bars represent the standard error derived from two independent experiments. Control unwounded stem tissue represented by 0 hr time point.

FIG. 4a. Wound-Inducible Accumulation of PERK1 mRNA in Brassica napus Leaf Disc Tissue.

The effects of a more localized wound defense response on the levels of PERK1 mRNA accumulation was investigated using wounded leaf discs. Total RNA was extracted from leaf discs at various time intervals, subjected the Northern blot analysis and probed with the full length PERK1 cDNA (bold-face arrow). The blot was reprobed with cyclophilin as an internal control for even loading (open-face arrow). The graph represents the steady state levels of PERK1 mRNA in response to wounding corrected against levels of cyclophilin expression. Control unwounded leaf tissue is represented by the 0 hr time point.

FIG. 4b. Wound-Inducible Accumulation of PERK1 mRNA in Brassica napus Leaf and Stem Tissue.

(A) Fully expanded leaves were wounded by rubbing the undersides with abrasive sand paper. Total RNA was extracted from the leaf at various time intervals after treatment, subjected the Northern blot analysis and probed with the full length PERK1 cDNA (bold-face arrow). The blot was reprobed with cyclophilin as an internal control for even loading (open-face arrow). The graph represents the steady state levels of PERK1 mRNA in response to wounding corrected against levels of cyclophilin expression. Control unwounded leaf tissue is represented by the 0 hr time point.

(B) Northern blot analysis showing a time-course induction of PERK1 mRNA accumulation in stem wounded by rubbing with abrasive sand paper. Total RNA harvested at the indicated time points was blotted and hybridized against the full length PERK1 cDNA (bold-face arrow). The cyclophilin loading control (open-face arrow) was used to normalize levels of PERK1 mRN accumulation represented graphically. Control unwounded stem tissue represented by 0 hr time point.

FIG. 4c. Wound-Inducible Accumulation of PERK1 mRNA in Brassica napus Root Tissue.

Root tissue from hydroponically grown B. napus plants was used to investigate whether levels of PERK1 mRNA increase in response to a wounding stimulus. Roots were wounded by slicing tissue into 3 cm sections and incubating on filter paper moistened with 20 mM phosphate buffer supplemented with chloramphenicol. Total RNA extracted at various time intervals after treatment, was subjected the Northern blot analysis and probed with the full length PERK1 cDNA (bold-face triangle). The blot was reprobed with cyclophilin as an internal control for even loading (open-face triangle). The graph represents the expression profile of PERK1 in response to wounding corrected against levels of cyclophilin expression. Control unwounded root tissue is represented by 0 hr time point

FIG. 5. Effects of 50 micromolar Methyl Jasmonate (MeJA) on PERK1 mRNA Accumulation in Treated Brassica napus Leaf and Stem Tissue.

(A) Brassica napus plants were thoroughly sprayed with a 50 micromolar MeJA solution, and leaf tissue subsequently harvested at different time intervals after treatment. Total RNA prepared from treated leaf tissue was subjected to Northern blot analysis and probed with full length PERK1 cDNA (open-face triangle). Control plant (0 hr) was treated with the carrying solution minus the chemical inducer (0.1% [v/v ] ethanol for MeJA). The blot was reprobed with cyclophilin used as an internal control for even loading (bold-face triangle). The graph represents a corrected profile for the levels of PERK1 mRNA accumulation in response to treatment with MeJA normalized against levels of cyclophilin expression.

(B) Northern blot showing a time-course induction of PERK1 mRNA accumulation in MeJA treated stem tissue. Total RNA harvested at the indicated time points was blotted and hybridized against the full length PERK1 coding sequence (open-face triangle). The cyclophilin loading control (bold-face triangle) was used to normalize levels of PERK1 mRNA accumulation represented graphically.

FIG. 6. Effects of 4 mM Salicylic Acid (SA) on PERK1 mRNA Accumulation in Treated Brassica napus Leaf and Stem Tissue.

(A) Brassica napus plants were thoroughly sprayed with a 4 mM SA solution, and leaf tissue subsequently harvested at different time intervals after treatment. Total RNA prepared from treated leaf tissue was subjected to Northern blot analysis and probed with full length PERK1 cDNA (open-face triangle). Control plant (0 hr) was treated with the carrying solution minus the chemical inducer (5 mM phosphate buffer, pH 7). The blot was reprobed with cyclophilin used as an internal control for even loading (open-face triangle). The graph represents a corrected profile for the levels of PERK1 mRNA accumulation in response to treatment with SA normalized against levels of cyclophilin expression.

(B) Northern blot showing a time-course induction of PERK1 mRNA accumulation in SA treated stem tissue. Total RNA harvested at the indicated time points was blotted and hybridized against the full length PERK1 coding sequence (open-face triangle). The cyclophilin loading control (bold-face triangle) was used to normalize levels of PERK1 mRNA accumulation represented graphically.

FIG. 7. B. napus leaf tissue was used to investigate whether levels of PERK1 mRNA increase in response to treatment with a fungal pathogen Sclerotinia sclerotiorum. Brassica napus leaves were excised from the plant, placed in orchid tubes with water and incubated in closed aluminum trays at room temperature under fluorescent light to generate a humid environment. The leaves were inoculated with fungal agar plugs and incubated for the indicated time points, after which the tissue was harvested and immediately frozen in liquid nitrogen for further analysis. Control time points were inoculated with agar plugs only. Total RNA was extracted and Northern blot analysis was subsequently performed. Blots were probed with the full length PERK1 cDNA and then with cyclophilin as a control for even loading. Throughout the experiment, it was noted that the fungal agar plugs began to adhere well to the tissue 7 hrs following inoculation and by 15 hrs post-inocluation macroscopic lesions were apparent on the surface of the leaves. The corrected profile for PERK1 mRNA accumulation in response to this fungal pathogen treatment shows that there was a 2.5 fold induction in the levels of PERK1 mRNA 10 hrs following treatment.

FIG. 8. Proposed pathway mediating PERK1 expression in response to wounding, MeJA and SA treatments.

FIG. 9. Analysis of Kinase Activity and Phosphoamino Acid Analysis of Recombinant PERK1 Protein. (A) Purification of recombinant mutant (K-E) and wild type catalytic domain fusion proteins. Extracts of E. coli cells harboring the fusion proteins were purified on MBP amylose resin. Proteins were visualized by Coomassie blue staining. The mutant CD fusion protein is present in lane 1 and the wild type CD fusion protein is represented in lane 2.

(B) Western blot analysis of recombinant fusion proteins. Western blot analysis using an anti-MBP antibody was performed on purified Cdmut_{K-E} and CDwt to confirm the identity of these proteins. Lane 1 represents detection of the mutant fusion protein and the signal obtained in lane 2 verifies the identity of the wild type fusion protein.

(C) Autoradiogram of autophosphorylation assay of recombinant PERK1 CDwt and Cdmut_{K-E} fusion proteins. Detection of a signal in lane 1 proves that the wild type catalytic domain of PERK1 is capable of autophosphorylation. Absence of a signal for the Cdmut_{K-E} in lane 2 confirms that the result obtained for CDwt is specific to the kinase activity of PERK1 and not due to bacterial kinase contamination.

(D) Phosphoamino acid analysis of autophosphorylated PERK. γ³²P-labeled CDwt was hydrolyzed with HCl and subjected to two-dimensional electrophoresis. Presence of radiolabeled phospho-serine (pS) and phospho-threonine (pT) confirms that PERK1 encodes an active protein kinase with serine/threonine substrate specificity.

FIG. 10. Shows sequence identity of PERK1 to polypeptides from the Arabidopsis genome sequencing project.

FIG. 11. Shows the nucleic acid molecule of [SEQ ID NO.: 6] and the amino acid sequence of [SEQ ID NO.: 7].

In a preferred embodiment, the figure shows the sequence of the predicted Arabidopsis gene—Accession number AAC98010

A) Genomic Sequence. The predicted open reading frame is underlined. The start codon (ATG) and stop codon (TGA) are double underlined.

B) Translation of the predicted open reading frame. The transmembrane domain is underlined.

FIG. 12. Shows the nucleic acid molecule of [SEQ ID NO.: 8] and the amino acid sequence of [SEQ ID NO.: 9].

In a preferred embodiment, the figure shows the sequence of the predicted Arabidopsis gene—Accession number AAD15491

A) Genomic Sequence. The predicted open reading frame is underlined. The start codon (ATG) and stop codon (TGA) are double underlined.

B) Translation of the predicted open reading frame. The transmembrane domain is underlined.

FIG. 13. Shows the nucleic acid molecule of [SEQ ID NO.: 10] and the amino acid sequence of [SEQ ID NO.: 11].

In a preferred embodiment, the figure shows the sequence of the predicted Arabidopsis gene—Accession number CAA18823.

A) Genomic Sequence. The predicted open reading frame is underlined. The start codon (ATG) and stop codon (TGA) are double underlined.

B) Translation of the predicted open reading frame. The transmembrane domain is underlined.

FIG. 14. Shows the nucleic acid molecule of [SEQ ID NO.: 12] and the amino acid sequence of [SEQ ID NO.: 13].

In a preferred embodiment, the figure shows the sequence of the predicted Arabidopsis gene—Accession number CAA18590

A) Genomic Sequence. The predicted open reading frame is underlined. The start codon (ATG) and stop codon (TGA) are double underlined.

B) Translation of the predicted open reading frame. The transmembrane domain is underlined.

FIG. 15. Schematic representation of the 35S:PERK1construct cloned in pCAMBIA 2301 plant transformation vector. Transgenic plants were selected on the basis of kanamycin resistance conferred by the neomycin phosphotransferase II (NPTII) gene. 35S CaMV P, cauliflower mosaic virus promoter; Nost, nopaline synthase terminator; GUS, β-glucoronidase. The presence of GUS in this construct enables the screening of transgenic plants.

FIG. 16. Phenotypes of transgenic A. thaliana plants overexpressing PERK1.

A. Northern blot and RT-PCR analysis on G₁ plants. Total RNA extracted from leaf tissue was subjected to RNA blot analysis and blots were probed with full length [α-³²P]ATP labeled PERK1 cDNA. Wt is the control—untransformed Arabidopsis plant. S2, S4, S10 are three different transgenic Arabidopsis lines overexpressing the PERK1 cDNA (note the stronger signal in these lanes compared to wt). B, Transgenic plants overexpressing PERK1 cDNA showed enhanced growth, secondary branching and fertility compared to wild-type Arabidopsis plants. wt, wild-type control—untransformed Arabidopsis plant; T, transgenic Arabidopsis plant overexpressing PERK1.

FIG. 17. Pistil phenotypes of transgenic A. thaliana plants overexpressing PERK 1.

The pistils of the fertile plants exhibited elongated papillae at the surface of the stigma (solid arrow) when compared to the stigmatic surface of the wild-type Arabidopsis plant. wt, wild-type control—untransformed Arabidopsis plant; T, transgenic Arabidopsis plant overexpressing PERK 1.

FIG. 18. Analysis of the overexpressing PERK1 lines. A, Table showing the average seeds no/siliques and the average dry seed weight/plant (mg). B, Bar graph represents the average height (cm) of the S2, S4 and S10 overexpressing transgenic Arabidopsis PERK1 lines compared to wild-type Arabidopsis plants.

FIG. 19. Membrane localization of a PERK1-GFP fusion protein.

In order to show the subcellular localization of PERK1 in vivo, a C-terminal GFP-PERK1 construct was engineered and transiently expressed under the control of the 35 CaMV promoter in onion epidermal cells by biolistic bombardment. Onion epidermal cells were placed on solid MS medium and bombarded with 5 μg of the fusion constructs. Following bombardment (˜24 hr), cells were examined using a Zeiss Axioskop2 MOT fluorescent microscope. Immunofluorescence images illustrate the subcellular localization of GFP-tagged PERK1. A. Onion epidermal cells transiently expressing the GFP control exhibit nuclear and cytoplasmic green fluorescence characteristic of the GFP localization (FIG. 19a). B. Delineation of transformed onion epidermal cell visualized in a DIC image. C. Onion epidermal cell transiently expressing PERK-GFP re-directs green fluorescence to periphery of cell consistent with the proposed localization of PERK1 to the plasma membrane (FIG. 19c). D. Corresponding DIC image outlining boundaries of the transformed cell as indicated by open face arrow. E. In order to differentiate between the plasma membrane and the cell wall, PERK1-GFP transformed cells were treated with 0.8M mannitol that is known to induce plasmolysis which creates a negative osmotic pressure resulting in the release of intracellular fluids and in the internalization of the plasma membrane along with cellular organelles and leaves the cell wall unchanged. FIGS. 19e and 19f illustrate fluoresence and DIC images of a plasmolysed cell, in which the PERK1-GFP fluorescence at the plasma membrane was internalized or pulled away from the cell wall as indicated by the arrows. Therefore, PERK 1 is localized to the plasma membrane as predicted for a receptor kinase.

### DETAILED DESCRIPTION OF THE INVENTION

In this study, we report the isolation and preliminary characterization of PERK1 nucleic acid molecules and polypeptides, and in particular PERK1 cDNA which encodes a novel receptor-like protein kinase in Brassica napus. PERK polypeptides represent a novel class of receptor kinases in higher plants. We show the role of PERK polypeptides in plant defense and in plant growth and development.

Protein kinases play important roles in plant defense (Zhou et al., 1995; Usami et al., 1995; Suzuki and Shinhi, 1995). Significant homology is shared between the extracellular domain of PERK1 and both extensin and proline rich proteins. PERK1 mediates plant responses to mechanical wounding (for example insect attack) and pathogen attack. PERK polypeptides preferably include a catalytic domain. PERK polypeptides are also preferably signaling molecules associated with the cell wall via their extensin-like extracellular domain and involved in the transduction of extracellular stimuli (e.g. wounding, pathogen attack) into an intracellular response through a transmembrane domain and a cytoplasmic kinase domain, thereby bridging the cell wall—plasma membrane continuum.

In general, plants challenged by mechanical wounding or pathogen attack induce rapid expression of genes (i.e. proteinase inhibitor (pin) and pathogenesis related (PR) genes respectively) that are expressed locally as well as systemically in unaffected parts of the plant (Yang et al., 1997). Increased levels of extensin transcripts as a result of mechanical wounding have been well established in many other systems (Sauer et al., 1990; Shirsat et al., 1996). For example, in Brassica napus leaf and stem tissue, wound induction of PERK1 mRNA accumulation is a very rapid response detectable within 15 min following injury (FIGS. 4, 4a-b). Increased levels of PERK1 mRNA were also detected in wounded root tissue within 5 min following treatment (FIG. 4c). MeJA (the methyl ester of the plant growth regulator jasmonic acid (JA)) is involved in the signal transduction pathway regulating gene activation upon wounding. Steady state levels of PERK1 mRNA remain unaffected by exogenously applied MeJA (FIG. 5) which shows that the inducibility of PERK1 by wounding occurs via a MeJA-independent pathway (FIG. 7). Studies conducted by Titarenko et al. (1997) addressing the role of JA in mediating wound responses support the existence of multiple distinct wound signal transduction pathways. Exogenously applied JA was able to induce only a subset of wound responsive genes in Arabidopsis which ultimately resulted in a stronger systemic accumulation in wounded plants. Conversely, a second set of wound responsive genes showing a stronger induction locally in wounded tissue showed no substantial accumulation upon JA treatment. In conjunction with the pattern of PERK1 mRNA accumulation in response to wounding and MeJA, it appears that plants respond to wounding by two distinct wound signal transduction pathways: one which does not require JA and is primarily responsible for gene activation in the vicinity of the wound site and the other which involves JA perception and activates gene expression both locally and systemically to the wound site (Titarenko et al., 1997).

Many of the inducible defense responses are not exclusive to mechanical wounding but are also initiated by pathogen attack. The similarity between responses to wounding and pathogen attack are not surprising since mechanical damage often precedes pathogen infection and conversely, mechanical damage may often result from a pathogen or insect attack (Truernit et al., 1996). Salicylic acid has been implicated in having an important role in the signal transduction pathway leading to systemic acquired resistance (SAR) (Penninckx et al., 1996). Steady state levels of PERK1 mRNA also accumulated in B. napus leaf and stem tissue upon exogenous application of 4 mM SA (FIG. 6). Collectively, the profiles of PERK1 mRNA accumulation in response to wounding, MeJA and SA are not entirely surprising. PERK1 induction is rapid in response to wounding (FIGS. 4, 4a-c) and the lack of PERK1 transcript accumulation in response to MeJA (FIG. 5) shows a pathway for wound mediated induction of PERK1 that is independent of MeJA (FIG. 7). The pronounced and rapid induction of PERK1 in response to exogenous SA (FIG. 6) supports other studies showing that SA is known to inhibit wound responsive genes that are regulated by a MeJA-dependent pathway (Peña-Cortés et al., 1993; Doares et al., 1995). Therefore, it is unlikely that both MeJA and SA would induce PERK1 mRNA accumulation given that these pathways are known to be antagonistic (Peña-Cortés et al., 1993). Nevertheless, the rapid induction of PERK1 during these treatments shows a role early on in a plant's defense signaling pathway.

Plant growth and development depends on the coordinated action of several phytohormones: Auxin, Gibberellin, Cytokinin, Abscisic Acid and Ethylene. Most of these hormones have been implicated in the control of diverse developmental processes. Some of these phytohormones are involved in increasing the cell wall plasticity, cell division, delayed senescence, and activation/degradation of target proteins (Leyser, 2001). The findings of phyotohormone Arabidopsis mutants have led to a model in which phytohormones control the abundance of the transcriptional regulators, the phytohormone efflux carriers and a range of other proteins (Leyser, 2001). This model provides an explanation as to how a single simple molecule can induce such diverse effects on different plant tissues. The focus is now on identifying the initial receptor protein involved in the perception of phytohormones and the targets of the signaling pathways activated in response to phytohormones (Reed, 2001). A major piece of missing information is how phytohormones influence the activity of the pathway. Phytohormones could regulate the phosphorylation state of the target proteins. There is some evidence for example that auxin signaling involving phosphorylation cascades. Recently, a MAPKKK from tobacco has been isolated that activates a MAP kinase cascade leading to the suppression of auxin-induced gene expression (Mockaitis and Howell, 2000). There is some other evidence that MAPKKK function downstream of other plasma membrane plant receptor kinases. The fact that overexpression of PERK1 affects many aspects pf plant growth and development shows that PERK1 acts downstream of one or more plant phytohormones to cause the increased growth and seed production.

Characterization of PERK1

Genomic Southern blot analysis under low and high stringency conditions revealed that PERK1 is a single copy gene in the Brassica genome (FIG. 2). PERK1 is ubiquitously expressed at high levels in stem, petal and pistil tissue and is less abundant in root, leaf and anther tissues (upper panel).

The deduced amino acid sequence of PERK1 shows that it is a transmembrane receptor kinase with a distinct extracellular, transmembrane and cytoplasmic domain (FIG. 1). The extracellular domain of PERK1 shows sequence similarity to plant cell wall proline-rich proteins and extensins which comprise a family of hydroxyproline-rich glycoproteins (HRGPs). Extensins are particularly abundant proteins in plant cell walls and are very rich in proline and serine as well as in combinations of valine, tyrosine, lysine and/or histidine residues. The distinctive characteristic of dicot extensins is their repetitive (Ser-Pro)₄ pentapeptide blocks. Although extensins are synthesized as soluble precursors, the majority of proline residues are hydroxylated and both the hydroxylated proline as well as the serine residues of these proteins are glycosylated by post-translational modifications (Cassab, 1998). When secreted to the plant cell wall, extensins become rapidly insoluble, presumably due to the formation of covalent isodityrosine bridges (Cassab, 1998). Although extensins have been proposed to be structural cell wall proteins and important in development, they have also been directly implicated in plant defense against mechanical wounding (Shirsat et al., 1996) and pathogen attack (Corbin et al., 1987; Showalter, 1993). The catalytic domain of PERK1 possesses all of the invariant residues necessary for kinase activity and sequence similarity in subdomains VI and VIII to amino acid consensus sequences characteristic of serine/threonine kinases shows a role for PERK1 in plant signal transduction (Hanks and Quinn, 1991). Given the similarity of PERK1 in the extracellular domain to the extensin family of cell wall proteins, PERK1 can detect changes to the cell wall through mechanical damage or pathogen attack and then pass the signal onto the cell. The cell can then respond to the attack with its defence mechanisms. Similarly to extensins, PERK1 also has a role in plant development. The extracellular domain of PERK1 can detect signals that regulate plant size, flowering, and seed production, and pass this signal onto the cell to respond by growth.

PERK nucleic acid molecules and proteins also have sequence identity and similarity to proline rich proteins as well. About 40% of PERK1's extracellular domain is comprised of proline. Below is a list of the Arabidopsis clones and their respective proline composition (Table below).

TABLE 1
% Prolines in Extracellular Domains

| Gene | % Proline in ECD |
|---|---|
| PERK1 | 56/137 = 41% |
| CA18590 | 105/279 = 38% |
| AAC98010 | 85/246 = 34% |
| CAA18823 | 51/179 = 28% |
| AAD15491 | 36/149 = 24% |

It is possible to use all or part of a proline rich domain from an extensin or a proline rich protein (or similar regions) to replace all or part of PERK1 's extracellular domain.

In summary, PERK1 is a unique plant protein in Brassica napus involved in wound and pathogen response, and in plant growth and development which physically links the cell wall and plasma membrane. PERK1 is involved in the general perception and subsequent transduction of a wound and/or pathogen stimulus, ultimately triggering a plant's defense mechanisms and conferring broad protection against such stimuli. PERK1 is also involved in the general perception and subsequent transduction of plant signals that control growth and development. Preliminary characterization showed that levels of PERK1 mRNA accumulate rapidly in response to wounding and SA. Further characterization of PERK1 induction with respect to changes in levels of phosphorylation provides additional evidence for the unequivocal role of PERK1 in plant defense. Furthermore, transgenic analysis of plants expressing altered levels of PERK1 confirms the involvement of PERK1 in wound and pathogen signaling. Transgenic analyses has confirmed the role of PERK1 in plant growth and development whereby overepxression of PERK1 leads to increased growth and seed production. PERK1 polypeptides and nucleic acid molecules may be isolated from the Brassicaceae including Arabidopsis, Brassica napus, Brassica rapa, Brassica juncea, Brassica oleracea, and other plants such as potato, tomato, tobacco, cotton, carrot, petunia, sunflower, strawberries, spinach, lettuce, rice, soybean, corn, wheat, rye, barley, sorgum and alfalfa.

PERK Nucleic Acid Molecules and Polypeptides

The invention relates to PERK nucleic acid molecules and polypeptides which increase wounding resistance and pathogen resistance in cells and plants, and increase growth and seed production in plants. These polypeptides preferably include an extracellular domain, a transmembrane domain and a cytoplasmic domain. The cytoplasmic domain preferably includes a region with kinase activity. The kinase activity is involved in cellular signaling. The PERK nucleic acid molecules which encode PERK polypeptides are particularly useful for producing transgenic plants which have increased wounding and pathogen resistance compared to a wild type plant and for producing transgenic plants that have increased growth and seed production.

It will also be apparent that there are polypeptide and nucleic acid molecules from other organisms, such as those listed previously, that are similar to PERK polypeptides and nucleic acid molecules. PERK polypeptides are useful in increasing wounding and pathogen resistance in a cell, preferably a plant cell, and for increased growth and seed production in plants because they include extensin-like and proline-rich domains (this refers to a plurality of domains including multiple proline residues, which are preferably similar to those found in extensins), such as SPPPP, SPP and PPP which are capable of being hydroxylated in response to wounding or pathogen attack. Once hydroxylated, extensins become rapidly insoluble which strengthens the cell wall and in response to pathogen attack helps agglutinate or prevent the spread of the pathogen to neighbouring plant cells. These same prolines are also responsible for detecting plant signals that control growth and seed production.

The PERK nucleic acid molecules and polypeptides, as well as their role in plants were not known before this invention. The ability of these compounds to increase wounding and pathogen resistance of transgenic host cells (particularly plant cells) and transgenic plants compared to wild type cells and plants was unknown. The ability of these compounds to increase growth and seed production was unknown.

All nucleotides and polypeptides which are suitable for use in the methods of the invention, such as the preparation of transgenic host cells or transgenic plants, are included within the scope of the invention. Genomic clones or cDNA clones are preferred for preparation of transgenic cells and plants.

In a preferred embodiment, the invention relates to a cDNA encoding PERK polypeptides from Brassica napus. The cDNA sequence and the corresponding amino acid sequence for PERK1 is presented in FIG. 1. The invention also includes splice variants of the nucleic acid molecules as well as polypeptides produced from the molecules.

Characterization of Nucleic Acid Molecules and Polypeptides

In one variation, the invention includes DNA sequences (and the corresponding polypeptide) including at least one of the sequences shown in FIG. 1 in a nucleic acid molecule of preferably about: less than 1000 base pairs, less than 1250 base pairs, less than 1500 base pairs, less than 1750 base pairs, less than 2000 base pairs, less than 2250 base pairs, less than 2500 base pairs, less than 2750 base pairs or less than 3000 base pairs.

The coding region of the PERK1 nucleic acid molecule is as follows:

TABLE 2

| | Start Nucleotide | End Nucleotide |
|---|---|---|
| | [brackets show | [brackets show |
| | corresponding amino | corresponding |
| Nucleic Acid Molecule | acid nos.] | amino acid nos.] |
| PERK1 (coding region | 1 (1) | 1944 (648) |
| only) | | |
| PERKi Extracellular | 1 (1) | 411 (137) |
| Domain | | |
| PERK1 Transmembrane | 412 (138) | 480 (160) |
| Domain | | |
| PERK1 Cytoplasmic | 481 (161) | 1944 (648) |
| Domain | | |
| PERK1 Kinase region | Same as | Same as |
| | cytoplasmic | cytoplasmic |
| | domain | domain |

It will be apparent that these may be varied, for example, by shortening the 5′ untranslated region or shortening the nucleic acid molecule so that the 3′ end nucleotide is in a different position.

The discussion of the nucleic acid molecules, sequence identity, hybridization and other aspects of nucleic acid molecules included within the scope of the invention is intended to be applicable to either the entire nucleic acid molecule in FIG. 1 or the coding region of this molecule, shown in Table 2. One may use the entire molecule in FIG. 1 or only the coding region. Other possible modifications to the sequence are apparent.

Southern Blot Analysis shows that PERK1 is present as a single copy gene in Brassica. A Northern blot showed that PERK1 polypeptide was expressed in all tissues examined (root, stem, leaf, petal, anther and pistil). It is highly expressed in the stem, petal and pistil tissues and to a lower extent in the root, leaf and anther tissues.

The PERK1 Nucleic Acid Molecule and Polypeptide are Conserved in Plants

Sequence Identity

This is the first isolation of a nucleic acid molecule encoding a PERK polypeptide from plant species. Nucleic acid sequences having sequence identity to the PERK1 sequence are found in other species of Brassica such as Brassica rapa, Brassica juncea, and Brassica oleracea as well as other plants such as Arabidopsis, potato, tomato, tobacco, cotton, carrot, petunia, sunflower, strawberries, spinach, lettuce, rice, soybean, corn, wheat, rye, barley, sorgum and alfalfa. Sequences from Brassica napus and other plants are collectively referred to as “PERK” nucleic acid sequences and polypeptides. We isolate PERK nucleic acid molecules from plants having nucleic acid molecules that are similar to those in Brassica napus, such as beet, tomato, rice, cucumber, radish and other plants including Arabidopsis, potato, tobacco, cotton, carrot, petunia, sunflower, strawberries, spinach, lettuce, soybean, corn, wheat, rye, barley, sorgum and alfalfa. and using techniques described in this application. The invention includes methods of isolating these nucleic acid molecules and polypeptides as well as methods of using these nucleic acid molecules and polypeptides according to the methods described in this application, for example those used with respect to PERK1.

Table 3 below shows several sequences with sequence identity and sequence similarity to the PERK1 polypeptide. Where polypeptides are shown, a suitable corresponding DNA encoding the polypeptide will be apparent. These sequences code for polypeptides similar to portions of PERK1 polypeptide. The sequences in Table 3 are useful to make probes to identify full length sequences or fragments (from the listed species or other species). They are useful to screen for functionally related cDNAs and genes. They are also useful to screen other tissues to see if they include all or part of the shown EST or similar sequences. The invention also relates to nucleic acid molecules including these EST sequences. In particular, the invention includes an isolated nucleic acid comprising a sequence that hybridizes under moderate or stringent conditions to SEQ ID NOS: 1 or 3 or the complements thereof. The invention also includes an isolated nucleic acid molecule comprising a sequence having at least about: >60%, >70%, >80% or >90%, more preferably at least about: >95%, or >99% sequence identity to a sequence in Table 3. One skilled in the art would be able to design a probe based on a polypeptide or peptide fragment. The invention includes nucleic acid molecules of about: 10 to 50 nucleotides, 50 to 200 nucleotides, 200 to 500 nucleotides, 500 to 1000 nucleotides, 1000 to 1500 nucleotides, 1500 to 1700 nucleotides, 1700 to 2000 nucleotides, 2000 to 2500 nucleotides or at least 2500 nucleotides and which include all or part of the sequences (or corresponding nucleic acid molecule) in Table 3. The invention also includes a nucleic acid molecule including the sequences in Table 3 which encodes peptides and polypeptides of about: 10 to 50 amino acids, 50 to 200 amino acids, 200 to 500 amino acids, 500 to 750 amino acids or at least 750 amino acids. Possible modifications to these sequences will also be apparent. The polypeptide and nucleic acid molecules are also useful in research experiments or in bioinformatics to locate other sequences. The nucleic acid molecules and polypeptides preferably provide PERK activity.

TABLE 3

| Organism | Accession No. |
|---|---|
| Arabidopsis thaliana | AAC 98010 (FIG. 11) |
| Arabidopsis thaliana | AAD 15491 (FIG. 12) |
| Arabidopsis thaliana | CAA 18823 (FIG. 13) |
| Arabidopsis thaliana | CAA 18590 (FIG. 14) |

The regions of importance include the extracellular domain (ECD), transmembrane domain (TMD), and the catalytic domain Amino acid positions are as follows:

AAC98010—ECD: 1-247; TMD: 248-267; CD: 268-732

AAD15491—ECD:1-149; TMD—150-171; CD—172-634

CAA18823—ECD:1-179; TMD—180-194; CD—195-675

CAA18590—ECD:1-279; TMD—280-302; CD—303-732

The invention includes the nucleic acid molecules from other plants as well as methods of obtaining the nucleic acid molecules by, for example, screening a cDNA library or other DNA collections with a probe of the invention (such as a probe comprising at least about: 10 or preferably at least 15 or 30 nucleotides of PERK1 and detecting the presence of a PERK nucleic acid molecule. Another method involves comparing the PERK1 sequences to other sequences, for example using bioinformatics techniques such as database searches or alignment strategies, and detecting the presence of a PERK nucleic acid molecule or polypeptide. The invention includes the nucleic acid molecule and/or polypeptide obtained according to the methods of the invention. The invention also includes methods of using the nucleic acid molecules, for example to make probes, in research experiments or to transform host cells or make transgenic plants. These methods are as described below.

The polypeptides encoded by the PERK nucleic acid molecules in other species will have amino acid sequence identity to the PERK1 sequence. Sequence identity may be at least about: >20%, >25%, >28%, >30%, >35%, >40%, >50% to an amino acid sequence shown in FIG. 1 (or a partial sequence thereof). Some polypeptides may have a sequence identity of at least about: >60%, >70%, >80% or >90%, more preferably at least about: >95%, >99% or >99.5% to an amino acid sequence in FIG. 1 (or a partial sequence thereof). The extensin-like extracellular domains are preferably at least: 30%, 35%, 40% or 45% similar to a plant extensin cell wall protein extracellular domain. The proline rich domains preferably contain at least: 20%, 25%, 30%, 35%, 40 or 45% proline. At4g33970, At2g14890, At4g13340, At1g62440, At4g22470, At1g12040, At1g76930, At4g13390 and At2g43150 are examples of extensins that may be compared to PERK1.

Identity is calculated according to methods known in the art. Sequence identity (nucleic acid and protein) is most preferably assessed by the algorithm of the Fasta 3 program, using the following default parameter settings: gap penalty (open)=−12 (protein)-16 (DNA), gap penalty (extension)=−2 (protein)-4 (DNA), protein weight matrix=BLOSUM 62. (The reference for FASTA 3 is W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA” Methods in Enzymology 183:63-98). The invention also includes modified polypeptide from plants which have sequence identity at least about: >20%, >25%, >28%, >30%, >35%, >40%, >50%, >60%, >70%, >80% or >90% more preferably at least about >95%, >99% or >99.5%, to the PERK sequence in FIG. 1 (or a partial sequence thereof). Modified polypeptide molecules are discussed below. Preferably about: 1, 2, 3, 4, 5, 6 to 10, 10 to 25, 26 to 50 or 51 to 100, or 101 to 250 nucleotides or amino acids are modified.

Nucleic Acid Molecules and Polypeptides Similar to PERK1

Those skilled in the art will recognize that the nucleic acid molecule sequences in FIG. 1 are not the only sequences which may be used to provide increased PERK activity in plants. The genetic code is degenerate so other nucleic acid molecules which encode a polypeptide identical to an amino acid sequence in FIG. 1 may also be used. The sequence of the other nucleic acid molecules of this invention may also be varied without changing the polypeptide encoded by the sequence. Consequently, the nucleic acid molecule constructs described below and in the accompanying examples for the preferred nucleic acid molecules, vectors, and transformants of the invention are merely illustrative and are not intended to limit the scope of the invention.

The sequences of the invention can be prepared according to numerous techniques. The invention is not limited to any particular preparation means. For example, the nucleic acid molecules of the invention can be produced by cDNA cloning, genomic cloning, cDNA synthesis, polymerase chain reaction (PCR), or a combination of these approaches (Current Protocols in Molecular Biology (F. M. Ausbel et al., 1989).). Sequences may be synthesized using well known methods and equipment, such as automated synthesizers. Nucleic acid molecules may be amplified by the polymerase chain reaction. Polypeptides may, for example, be synthesized or produced recombinantly.

Sequence Identity

The invention includes modified nucleic acid molecules with a sequence identity at least about: >17%, >20%, >30%, >40%, >50%, >60%, >70%, >80% or >90% more preferably at least about >95%, >99% or >99.5%, to a DNA sequence in FIG. 1 (or a partial sequence thereof). Preferably about 1, 2, 3, 4, 5, 6 to 10, 10 to 25, 26 to 50 or 51 to 100, or 101 to 250 nucleotides or amino acids are modified. Identity is calculated according to methods known in the art. Sequence identity is most preferably assessed by the algorithm of the FASTA 3 program. For example, if a nucleotide sequence (called “Sequence A”) has 90% identity to a portion of the nucleotide sequence in FIG. 1, then Sequence A will be identical to the referenced portion of the nucleotide sequence in FIG. 1, except that Sequence A may include up to 10 point mutations, such as substitutions with other nucleotides, per each 100 nucleotide of the referenced portion of the nucleotide sequence in FIG. 1. Nucleotide sequences functionally equivalent to the PERK1 sequence can occur in a variety of forms as described below. Polypeptides having sequence identity may be similarly identified.

The polypeptides encoded by the homologous PERK nucleic acid molecule in other species will have amino acid sequence identity at least about: >20%, >25%, >28%, >30%, >40% or >50% to an amino acid sequence shown in FIG. 1 (or a partial sequence thereof). Some plant species may have polypeptides with a sequence identity of at least about: >60%, >70%, >80% or >90%, more preferably at least about: >95%, >99% or >99.5% to all or part of an amino acid sequence in FIG. 1 (or a partial sequence thereof). Identity is calculated according to methods known in the art. Sequence identity is most preferably assessed by the FASTA 3 program. Preferably about: 1, 2, 3, 4, 5, 6 to 10, 10 to 25, 26 to 50 or 51 to 100, or 101 to 250 nucleotides or amino acids are modified. The extensin-like extracellular domains are preferably at least: 30%, 35%, 40% or 45% similar to a plant extensin cell wall protein extracellular domain. The proline rich domains preferably contain at least: 20%, 25%, 30%, 35%, 40 or 45% proline.

The invention includes nucleic acid molecules with mutations that cause an amino acid change in a portion of the polypeptide not involved in providing PERK activity or an amino acid change in a portion of the polypeptide involved in providing PERK activity so that the mutation increases or decreases the activity of the polypeptide.

Hybridization

Other functional equivalent forms of the PERK nucleic acid molecules encoding nucleic acids can be isolated using conventional DNA-DNA or DNA-RNA hybridization techniques. These nucleic acid molecules and the PERK sequences can be modified without significantly affecting their activity.

The present invention also includes nucleic acid molecules that hybridize to one or more of the sequences in FIG. 1 (or a partial sequence thereof) or their complementary sequences, and that encode peptides or polypeptides exhibiting substantially equivalent activity as that of an PERK polypeptide produced by the DNA in FIG. 1. Such nucleic acid molecules preferably hybridize to all or a portion of PERK or its complement or all or a portion of an EST of Table 3 under low, moderate (intermediate), or high stringency conditions as defined herein (see Sambrook et al. (Most recent edition) Molecular Cloning: A Laboratory Manual, Cold Spring Harbor Laboratory Press, Cold Spring Harbor, N.Y.; Ausubel et al. (eds.), 1995, Current Protocols in Molecular Biology, (John Wiley & Sons, NY)). The portion of the hybridizing nucleic acids is typically at least 15 (e.g. 20, 25, 30 or 50) nucleotides in length. The hybridizing portion of the hybridizing nucleic acid is at least 80% e.g. at least 95% or at least 98% identical to the sequence or a portion or all of a nucleic acid encoding a PERK polypeptide, or its complement. Hybridizing nucleic acids of the type described herein can be used, for example, as a cloning probe, a primer (e.g. a PCR primer) or a diagnostic probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions involve hybridizing at about: 2× SSC, 0.1% SDS at 50° C. High stringency conditions are: 0.1× SSC, 0.1% SDS at 65° C. Moderate stringency is about 1× SSC 0.1% SDS at 60 degrees Celsius. The parameters of salt concentration and temperature can be varied to achieve the optimal level of identity between the probe and the target nucleic acid.

The present invention also includes nucleic acid molecules from any source, whether modified or not, that hybridize to genomic DNA, cDNA, or synthetic DNA molecules that encode the amino acid sequence of a PERK polypeptide, or genetically degenerate forms, under salt and temperature conditions equivalent to those described in this application, and that code for a peptide, or polypeptide that has PERK activity. Preferably the polypeptide has the same or similar activity as that of a PERK1 polypeptide. A nucleic acid molecule described above is considered to be functionally equivalent to a PERK nucleic acid molecule (and thereby having PERK activity) of the present invention if the polypeptide produced by the nucleic acid molecule displays the following characteristics: The defining feature of PERK polypeptides is the presence of a proline-rich domain, followed by a transmembrane domain, followed by a kinase domain. When tested, the kinase domain has serine/threonine kinase activity.

The invention also includes nucleic acid molecules and polypeptides having sequence similarity taking into account conservative amino acid substitutions. Sequence similarity (and preferred percentages) are discussed below.

Modifications to Nucleic Acid Molecule or Polypeptide Sequence

Changes in the nucleotide sequence which result in production of a chemically equivalent or chemically similar amino acid sequences are included within the scope of the invention. Variants of the polypeptides of the invention may occur naturally, for example, by mutation, or may be made, for example, with polypeptide engineering techniques such as site directed mutagenesis, which are well known in the art for substitution of amino acids. For example, a hydrophobic residue, such as glycine can be substituted for another hydrophobic residue such as alanine. An alanine residue may be substituted with a more hydrophobic residue such as leucine, valine or isoleucine. A negatively charged amino acid such as aspartic acid may be substituted for glutamic acid. A positively charged amino acid such as lysine may be substituted for another positively charged amino acid such as arginine.

Therefore, the invention includes polypeptides having conservative changes or substitutions in amino acid sequences. Conservative substitutions insert one or more amino acids which have similar chemical properties as the replaced amino acids. The invention includes sequences where conservative substitutions are made that do not destroy PERK activity. The preferred percentage of sequence similarity for sequences of the invention includes sequences having at least about: 50% similarity to PERK1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has PERK activity. The invention also includes nucleic acid molecules encoding polypeptides, with the polypeptides having at least about: 50% similarity to PERK1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has PERK activity, to an amino acid sequence in FIG. 1 (or a partial sequence thereof) considering conservative amino acid changes, wherein the polypeptide has PERK activity. Sequence similarity is preferably calculated number of similar amino acids in a multiple alignment expressed as a percentage of the shorter of the two sequences in the alignment. The multiple alignment is preferably constructed using the algorithm of the FASTA 3 program, using the following parameter settings: gap penalty (open)=−12 (protein)-16 (DNA), gap penalty (extension)=−2 (protein)-4 (DNA), protein weight matrix=BLOSUM 62. (The reference for FASTA 3 is W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA” Methods in Enzymology 183:63-98).

Polypeptides comprising one or more d-amino acids are contemplated within the invention. Also contemplated are polypeptides where one or more amino acids are acetylated at the N-terminus. Those of skill in the art recognize that a variety of techniques are available for constructing polypeptide mimetics with the same or similar desired PERK activity as the corresponding polypeptide compound of the invention but with more favorable activity than the polypeptide with respect to solubility, stability, and/or susceptibility to hydrolysis and proteolysis. See, for example, Morgan and Gainor, Ann. Rep. Med. Chem., 24:243-252 (1989). Examples of polypeptide mimetics are described in U.S. Pat. No. 5,643,873. Other patents describing how to make and use mimetics include, for example in, U.S. Pat. Nos. 5,786,322, 5,767,075, 5,763,571, 5,753,226, 5,683,983, 5,677,280, 5,672,584, 5,668,110, 5,654,276, 5,643,873. Mimetics of the polypeptides of the invention may also be made according to other techniques known in the art. For example, by treating a polypeptide of the invention with an agent that chemically alters a side group by converting a hydrogen group to another group such as a hydroxy or amino group. Mimetics preferably include sequences that are either entirely made of amino acids or sequences that are hybrids including amino acids and modified amino acids or other organic molecules.

The invention also includes hybrid nucleic acid molecules and polypeptides, for example where a nucleotide sequence from one species of plant is combined with a nucleotide sequence from another sequence of plant, mammal, bacteria or yeast to produce a fusion polypeptide. The invention includes a fusion protein having at least two components, wherein a first component of the fusion protein comprises a polypeptide of the invention, preferably a full length PERK polypeptide. The second component of the fusion protein preferably comprises a tag, for example GST, an epitope tag or an enzyme. The fusion protein may comprise lacZ.

The invention also includes polypeptide fragments of the polypeptides of the invention which may be used to confer PERK activity if the fragments retain activity. The invention also includes polypeptides fragments of the polypeptides of the invention which may be used as a research tool to characterize the polypeptide or its activity. Such polypeptides preferably consist of at least 5 amino acids. In preferred embodiments, they may consist of 6 to 10, 11 to 15, 16 to 25, 26 to 50, 51 to 75,76 to 100 or 101 to 250 amino acids of the polypeptides of the invention (or longer amino acid sequences). The fragments preferably have PERK activity. Fragments may include sequences with one or more amino acids removed, for example, C-terminus amino acids in a PERK sequence.

The invention also includes a composition comprising all or part of an isolated PERK nucleic acid molecule (preferably PERK1) of the invention and a carrier, preferably in a composition for plant transformation. The invention also includes a composition comprising an isolated PERK polypeptide (preferably PERK1) and a carrier, preferably for studying polypeptide activity.

Recombinant Nucleic Acid Molecules

The invention also includes recombinant nucleic acid molecules preferably a PERK1 sequence of FIG. 1 comprising a nucleic acid molecule of the invention and a promoter sequence, operatively linked so that the promoter enhances transcription of the nucleic acid molecule in a host cell (the nucleic acid molecules of the invention may be used in an isolated native gene or a chimeric gene, for example, where a nucleic acid molecule coding region is connected to one or more heterologous sequences to form a gene. The promoter sequence is preferably a constitutive promoter sequence or an inducible promoter sequence, operatively linked so that the promoter enhances transcription of the DNA molecule in a host cell. The promoter may be of a type not naturally associated with the cell such as a super promoter, a 35S cauliflower mosaic virus promoter, a chemical inducible promoter, a copper-inducible promoter, a steroid-inducible promoter and a tissue specific promoter.

A recombinant nucleic acid molecule for conferring PERK activity may also contain suitable transcriptional or translational regulatory elements. Suitable regulatory elements may be derived from a variety of sources, and they may be readily selected by one with ordinary skill in the art. Examples of regulatory elements include: a transcriptional promoter and enhancer or RNA polymerase binding sequence, a ribosomal binding sequence, including a translation initiation signal. Additionally, depending on the vector employed, other genetic elements, such as selectable markers, may be incorporated into the recombinant molecule. Markers facilitate the selection of a transformed host cell. Such markers include genes associated with temperature sensitivity, drug resistance, or enzymes associated with phenotypic characteristics of the host organisms.

Nucleic acid molecule expression levels are controlled with a transcription initiation region that regulates transcription of the nucleic acid molecule or nucleic acid molecule fragment of interest in a plant, bacteriaor yeast cell. The transcription initiation region may be part of the construct or the expression vector. The transcription initiation domain or promoter includes an RNA polymerase binding site and an mRNA initiation site. Other regulatory regions that may be used include an enhancer domain and a termination region. The regulatory elements described above may be from animal, plant, yeast, bacteria, fungus, virus or other sources, including synthetically produced elements and mutated elements.

Methods of modifying DNA and polypeptides, preparing recombinant nucleic acid molecules and vectors, transformation of cells, expression of polypeptides are known in the art. For guidance, one may consult the following U.S. Pat. Nos. 5,840,537, 5,850,025, 5,858,719, 5,710,018, 5,792,851, 5,851,788, 5,759,788, 5,840,530, 5,789,202, 5,871,983, 5,821,096, 5,876,991, 5,422,108, 5,612,191, 5,804,693, 5,847,258, 5,880,328, 5,767,369, 5,756,684, 5,750,652, 5,824,864, 5,763,211, 5,767,375, 5,750,848, 5,859,337, 5,563,246, 5,346,815, and WO9713843. Many of these patents also provide guidance with respect to experimental assays, probes and antibodies, methods, transformation of host cells and regeneration of plants, which are described below. These patents, like all other patents, publications (such as articles and database publications) in this application, are incorporated by reference in their entirety.

Host Cells Including a PERK Nucleic Acid Molecule

In a preferred embodiment of the invention, a plant or yeast cell is transformed with a nucleic acid molecule of the invention or a fragment of a nucleic acid molecule and inserted in a vector.

Another embodiment of the invention relates to a method of transforming a host cell with a nucleic acid molecule of the invention or a fragment of a nucleic acid molecule, inserted in a vector. The invention also includes a vector comprising a nucleic acid molecule of the invention. The nucleic acid molecules can be cloned into a variety of vectors by means that are well known in the art. The recombinant nucleic acid molecule may be inserted at a site in the vector created by restriction enzymes. A number of suitable vectors may be used, including cosmids, plasmids, bacteriophage, baculoviruses and viruses. Suitable vectors are capable of reproducing themselves and transforming a host cell. The invention also relates to a method of expressing polypeptides in the host cells. A nucleic acid molecule of the invention may be used to transform virtually any type of plant, including both monocots and dicots. The expression host may be any cell capable of expressing PERK, such as a cell selected from the group consisting of a seed (where appropriate), plant cell, bacterium, yeast, fungus, protozoa, algae, animal and animal cell.

Levels of nucleic acid molecule expression may be controlled with nucleic acid molecules or nucleic acid molecule fragments that code for anti-sense RNA inserted in the vectors described above.

Agrobacterium tumefaciens-mediated transformation, particle-bombardment-mediated transformation, direct uptake, microinjection, coprecipitation and electroporation-mediated nucleic acid molecule transfer are useful to transfer a PERK nucleic acid molecule into seeds (where appropriate) or host cells, preferably plant cells, depending upon the plant species. The invention also includes a method for constructing a host cell capable of expressing a nucleic acid molecule of the invention, the method comprising introducing into said host cell a vector of the invention. The genome of the host cell may or may not also include a functional PERK gene. The invention also includes a method for expressing a PERK polypeptide such as a PERK1 in FIG. 1 in the host cell or a plant, plant part, seed or plant cell of the invention, the method comprising culturing the host cell under conditions suitable for gene expression. The method preferably also includes recovering the expressed polypeptide from the culture.

The invention includes the host cell comprising the recombinant nucleic acid molecule and vector as well as progeny of the cell. Preferred host cells are fungal cells, yeast cells, bacterial cells, mammalian cells, bird cells, reptile cells, amphibious cells, microorganism cells and plant cells. Host cells may be cultured in conventional nutrient media. The media may be modified as appropriate for inducing promoters, amplifying genes or selecting transformants. The culture conditions, such as temperature, composition and pH will be apparent. After transformation, transformants may be identified on the basis of a selectable phenotype. A selectable phenotype can be conferred by a selectable marker in the vector.

Transgenic Plants and Seeds

Plant cells are useful to produce tissue cultures, seeds or whole plants. The invention includes a plant, plant part, seed, or progeny thereof including a host cell transformed with a PERK nucleic acid molecule such as a molecule in FIG. 1. The plant part is preferably a leaf, a stem, a flower, a root, a seed or a tuber.

The invention includes a transformed (transgenic) plant having increased PERK activity, the transformed plant containing a nucleic acid molecule sequence encoding for polypeptide activity and the nucleic acid molecule sequence having been introduced into the plant by transformation under conditions whereby the transformed plant expresses a PERK polypeptide in an active form.

The methods and reagents for producing mature plants from cells are known in the art. The invention includes a method of producing a genetically transformed plant which expresses PERK polypeptide such as a polypeptide in FIG. 1 by regenerating a genetically transformed plant from the plant cell, seed or plant part of the invention. The invention also includes the transgenic plant produced according to the method. Alternatively, a plant may be transformed with a vector of the invention.

The invention also includes a method of preparing a plant with increased PERK activity, the method comprising transforming the plant with a nucleic acid molecule which encodes a polypeptide of FIG. 1 or a polypeptide encoding a PERK polypeptide capable of increasing PERK activity in a cell, and recovering the transformed plant with increased PERK activity. The invention also includes a method of preparing a plant with increased PERK activity, the method comprising transforming a plant cell with a nucleic acid molecule such as a molecule of FIG. 1 which encodes a PERK polypeptide capable of increasing PERK activity in a cell.

Overexpression of PERK leads to an improved ability of the transgenic plants to resist wounding or pathogen damage. Overexpression of PERK has been demonstrated to lead to increased growth and seed production.

The plants whose cells may be transformed with a nucleic acid molecule of this invention and used to produce transgenic plants include, but are not limited to the

Target Plants:

Brassica napus, Brassica rapa, Brassica juncea, Brassica oleracea, or from the family Brassicaecae, Arabidopsis, potato, tomato, tobacco, cotton, carrot, petunia, sunflower, strawberries, spinach, lettuce, rice, soybean, corn, wheat, rye, barley, sorgum and alfalfa. Cereal plants including rye, barley and wheat may also be transformed with a PERK polypeptide, preferably PERK1.

In a preferred embodiment of the invention, plant tissue cells or cultures which demonstrate PERK activity (or increased PERK activity compared to wild type) are selected and plants are regenerated from these cultures. Methods of regeneration will be apparent to those skilled in the art (see Examples below, also). These plants may be reproduced, for example by cross pollination with a plant that does not have PERK activity. If the plants are self-pollinated, homozygous progeny may be identified from the seeds of these plants, for example, using genetic markers. Seeds obtained from the mature plants resulting from these crossings may be planted, grown to sexual maturity and cross-pollinated or self-pollinated.

Transgenic A. thaliana and B. napus plants overexpressing the PERK1 cDNA have been generated. Northern blot and RT-PCR analysis was performed on several transgenic A. thaliana lines overexpressing the PERK1 cDNA. Total RNA extracted from leaf tissue was subjected to RNA blot analysis and probed with the full length [a-32P]ATP labeled PERK1 cDNA which showed overexpression of PERK1 (FIG. 16).

The nucleic acid molecule is also incorporated in some plant species by breeding methods such as back crossing to create plants homozygous for the PERK nucleic acid molecule.

A plant line homozygous for the PERK nucleic acid molecule may be used as either a male or female parent in a cross with a plant line lacking the PERK nucleic acid molecule to produce a hybrid plant line which is uniformly heterozygous for the nucleic acid molecule. Crosses between plant lines homozygous for the PERK nucleic acid molecule are used to generate hybrid seed homozygous for the resistance nucleic acid molecule.

Fragments/Probes

Preferable fragments include 10 to 50, 50 to 100, 100 to 250, 250 to 500, 500 to 1000, 1000 to 1500, or 1500 or more nucleotides of a nucleic acid molecule of the invention. A fragment may be generated by removing a single nucleotide from a sequence in FIG. 1 (or a partial sequence thereof). Fragments may or may not encode a polypeptide having PERK activity.

The nucleic acid molecules of the invention (including a fragment of a sequence in FIG. 1 (or a partial sequence thereof) can be used as probes to detect nucleic acid molecules according to techniques known in the art (for example, see U.S. Pat. Nos. 5,792,851 and 5,851,788). The probes may be used to detect nucleic acid molecules that encode polypeptides similar to the polypeptides of the invention. For example, a probe having at least about 10 bases will hybridize to similar sequences under stringent hybridization conditions (Sambrook et al. 1989, Molecular Cloning, A Laboratory Manual, Cold Spring Harbor). Polypeptide fragments of PERK1 are preferably at least 8 amino acids in length and are useful, for example, as immunogens for raising antibodies that will bind to intact protein (immunogenic fragments). Typically the average length used for synthetic peptides is 8-16, 8 being the minimum, however 12 amino acids is commonly used.

Kits

The invention also includes a kit for conferring increased PERK activity to a plant or a host cell including a nucleic acid molecule of the invention (preferably in a composition of the invention) and preferably reagents for transforming the plant or host cell.

The invention also includes a kit for detecting the presence of PERK nucleic acid molecule (e.g. a molecule in FIG. 1), comprising at least one probe of the invention. Kits may be prepared according to known techniques, for example, see U.S. Pat. Nos. 5,851,788 and 5,750,653.

Antibodies

The invention includes an isolated antibody immunoreactive with a polypeptide of the invention. Antibodies are preferably generated against epitopes of native PERK1 or synthetic peptides of PERK1. The antibody may be labeled with a detectable marker or unlabeled. The antibody is preferably a monoclonal antibody or a polyclonal antibody. PERK antibodies can be employed to screen organisms containing PERK polypeptides. The antibodies are also valuable for immuno-purification of polypeptides from crude extracts.

Examples of the preparation and use of antibodies are provided in U.S. Pat. Nos. 5,792,851 and 5,759,788. For other examples of methods of the preparation and uses of monoclonal antibodies, see U.S. Pat. Nos. 5,688,681, 5,688,657, 5,683,693, 5,667,781, 5,665,356, 5,591,628, 5,510,241, 5,503,987, 5,501,988, 5,500,345 and 5,496,705. Examples of the preparation and uses of polyclonal antibodies are disclosed in U.S. Pat. Nos. 5,512,282, 4,828,985, 5,225,331 and 5,124,147.

The invention also includes methods of using the antibodies. For example, the invention includes a method for detecting the presence of a PERK polypeptide such as PERK1, by: a) contacting a sample containing one or more polypeptides with an antibody of the invention under conditions suitable for the binding of the antibody to polypeptides with which it is specifically reactive; b) separating unbound polypeptides from the antibody; and c) detecting antibody which remains bound to one or more of the polypeptides in the sample.

Research Tool

Cell cultures, seeds, plants and plant parts transformed with a nucleic acid molecule of the invention are useful as research tools. For example, one may obtain a plant cell (or a cell line,) that does not express PERK1, insert a PERK1 nucleic acid molecule in the cell, and assess the level of PERK1 expression and activity.

The PERK nucleic acid molecules and polypeptides including those in FIG. 1 are also useful in assays. Assays are useful for identification and development of compounds to inhibit and/or enhance polypeptide function directly.

Suitable assays may be adapted from, for example, U.S. Pat. No. 5,851,788.

Using Exogenous Agents in Combination with a Vector

The nucleic acid molecules of the invention may be used with other nucleic acid molecules that relate to plant protection, for example, extensin nucleic acid molecules. Host cells or plants may be transformed with these nucleic acid molecules.

PERK1 Activity

We show that PERK1 encodes a protein with kinase activity as its sequence predicts. The bacterially expressed catalytic domain fusion protein of PERK1 is tested for kinase activity (FIG. 9c). Furthermore, to ensure that the phosphorylation of the fusion protein was not a result of bacterial kinase activity, a mutated catalytic domain was also generated by site directed mutagenesis which introduced a single base pair substitution of a lysine residue to a glutamic acid residue (K→E). This mutation modifies the essential invariant lysine of subdomain II required for phospho-transfer and renders the kinase inactive. Both the wild-type and the mutated catalytic domains of PERK1 were cloned into the pMAL-c expression system, induced for protein production in the presence of IPTG and purified by affinity chromatography on MBP amylose resin (FIG. 9a). FIG. 9b is a Western blot to confirm the induction and purification of both the wild-type and mutated catalytic domain fusion proteins using an anti-MBP antibody. The wild-type fusion protein appears to be toxic in bacteria which compromises its inducibility and purification (FIG. 9a; lane 2). The mutated fusion protein is induced and purified more efficiently, perhaps due to the fact that it is no longer kinase active (FIG. 9a; lane 1).

FIG. 9c represents a kinase assay performed on affinity purified wild-type and mutated fusion proteins incubated in the presence of γ-32PdATP. Detection of a phosphoprotein only in lane 1 provides direct biochemical evidence that the wild-type catalytic domain of PERK1 encodes a functional protein kinase that is capable of autophosphorylation (Lane 1) and that the mutation successfully abolished kinase activity (Lane 2).

Since PERK1 is known to encode a protein with kinase activity, phosphoamino acid analysis was performed to determine the amino acid specificity of its autophosphorylating activity. The results in FIG. 9d demonstrate that PERK1 is phosphorylated on serine/threonine residues and is therefore a serine/threonine receptor kinase.

Transgenic Plants

An important focus of agricultural biotechnology research is in devising new strategies to combat crop losses due to plant diseases and pests. Advances in the development and improvement of plant transformation techniques has opened up new avenues for generating crops with enhanced resistance against disease and insect attack (i.e. mechanical wounding). Plants can now be engineered to express pathogen derived compounds that disrupt the infection process or alternatively a more desirable and perhaps effective approach is to enhance a plant's endogenous defense mechanisms. Furthermore, the downstream defense mechanisms are fairly well understood, therefore manipulation of some potential upstream signals that control the battery of defense proteins may be a promising strategy to engineer plants with enhanced and broad-spectrum resistance against both insect injury and pathogen attack. PERK1 functions as one such gene.

One approach to show the role of a particular gene in the regulation of gene expression in response to pathogen attack or mechanical wounding has been to generate transgenic plants constitutively expressing the respective cDNA in the sense [1-2] or antisense [3] orientations and examining the effects of wounding and pathogen attack on known downstream target genes involved in these processes. Generating plants (including plant parts and seeds) that overexpress the antisense PERK1 transcript shows the role of PERK1 in mediating a plant's defense response to both wounding and pathogen attack. By mechanically injuring, inoculating with pathogens (i.e. Sclerotinia sclerotiorum, Cylindrosporium concertricum, Phoma lingam) or treating with chemical elicitors and looking at the levels of downstream genes directly involved in these various processes, we directly implicate PERK1 in these pathways. For example, the expression of the functional PERK1 protein is abolished and induction of downstream genes is reduced, so PERK1 is an important upstream component of the pathway. PERK1 offers protection against wounding and pathogen attack, so plants overexpressing PERK1 in the sense orientation exhibit an accumulation of downstream target transcripts involved in these responses and ultimately enhanced resistance.

We score for a phenotype such as enhanced survival of plants overexpressing PERK1 compared to the enhanced susceptibility of plants overexpressing antisense PERK1 transcripts in response to pathogen treatment.

(References 1. Tang, X. et al. (1999). Overexpression of Pto activates defense responses and confers broad resistance. Plant Cell 11, 15-29; 2. Cao, H. et al. (1998). Generation of broad-spectrum disease resistance by overexpression of an essential regulatory gene in systemic acquired resistance. Proc. Natl. Acad. Sci. 95, 6531-6536; 3. Royo, J. et al. (1999). Antisense-mediated depletion of a potato lipoxygenase reduces wound induction of proteinase inhibitors and increases weight gain of insect pests. Proc. Natl. Acad. Sci. 96, 1146-1151.)

A second important focus of agricultural biotechnology research is in devising new strategies to increase plant growth and seed production to increase crop yields. We have accomplished this by overexpressing the PERK1 cDNA.

Transgenic PERK1 Brassica napus Plants

Upon generating transgenic plants constitutively expressing the PERK1 cDNA in the sense orientation, as described below, we examine the effects of wounding and pathogen attack on known downstream genes involved in these processes to confirm the involvement of PERK1 in these pathways. PERK1 is an upstream component of these pathways. It is involved in mediating a plant's defense response to both wounding and pathogen attack, and transgenic plants overexpressing the PERK1 cDNA in the sense orientation exhibit an increase in the expression of downstream target genes of these pathways. Furthermore, these plants exhibit an enhanced survival relative to a wild type plant in response to wounding and pathogen attack.

PERK1 is a signaling molecule in response to wounding and pathogen attack by generating transgenic plants expressing an HA-epitope tagged PERK1 protein at either the C or N terminus of the protein. Induction of PERK1 with respect to changes in levels of phosphorylation shows the role of PERK1 in these processes. The PERK1 protein is immunoprecipitated using the anti-HA antibody from total protein extracts prepared from B. napus tissue after treatment with a specific stimulus. This approach provides quantitative results for the levels of PERK1 phosphorylation in response to various stimuli.

Overexpression of PERK1 also increases plant growth and seed production. We increased expression levels of PERK1 in transgenic Arabidopsis thaliana plants, in one embodiment described below. We observed an enhanced growth phenotype with one advantage being increased seed production by twice as much as that observed for the wild-type plant based on total dry seed weight. We also overexpress the PERK1 cDNA in Brassica napus and other plants listed in this patent application to enhance the seed production and overall growth.

The enhanced growth phenotype observed in the transgenic PERK1 overexpressing A. thaliana plants shows the following:

1) increased height compared to wild-type plants (preferably at least 20 or at least 30% more);

2) increased number of branches (stem number) compared to wild type plants (preferably at least 100 or at least 200% more);

3) increased number of seed pods compared to wild type plants (preferably at least 50 or at least 60% more);

4) an increased seed production compared to wild-type plants (preferably at least 50 or at least 80% more). We determine that each seed pod produces more seeds than wild type; and

5) earlier flowering (preferably flowering at least 20% earlier) and later senescence (preferably flowering for a 50% longer period) compared to wild type plants.

Generation of PERK1 A. thaliana Transgenic Plants

Expression levels of PERK1 gene were altered in transgenic Arabidopsis thaliana plants using an overexpression strategy. The coding region of the PERK1 cDNA was cloned downstream of the 35S cauliflower mosaic virus (CaMV) promoter into the pCAMBIA 2301 plant transformation vector and transformed into Agrobacterium tumefaciens (strain GV3101 pmp90) (FIG. 15). Arabidopsis thaliana (Col-0) plants were transformed using a modified “floral dip” method from Clough and Bent. The resulting seeds resistant to kanamycin antibiotic were identified as transformants and later transplanted to soil and allowed to self. DNA and RNA blot analyses of overexpressing PERK1 transgenic plants identified three independent lines (S₂, S₄, S₁₀) harboring a single copy of the T-DNA and expressing a high level of the PERK1 mRNA (FIG. 16A). These lines were selfed until the third generation and homozygous plants within these lines were used for our analyses.

Analyses of PERK1 A. thaliana Transgenic Plants

Transgenic plants overexpressing the PERK1 gene exhibited several heritable traits including enhanced growth and fertility (FIG. 16B). The S₂, S₄ and S₁₀ independent lines show increased height and secondary branching compared to the wild-type plants (FIGS. 16B, 18B). The pistils of overexpressing PERK1 sense plants also exhibit an altered phenotype when compared to pistils of wild-type Arabidopsis plants, in that the papillae cells at the surface of the stigma appear elongated (FIG. 17). Furthermore, scanning electron micrograph experiments illustrate that the enhanced growth/height apparent in these overexpressing plants is the result of cell expansion and not due to increased cell division.

The overexpressing PERK1 lines showed an extended life cycle with the plants flowering earlier and senescing later than the wild-type plants. In addition, there was no decrease in the fertility or seed viability in these lines. The overexpressing PERK1 lines showed an increase in the total number of siliques (seed pods) per plant and an increase in the number of seeds per silique (FIG. 18A) resulting in an increase in the dry seed weight per plant (FIG. 18A). The dry seed weight per plant for the overexpressing PERK1 lines is approximately twice as much as for wild-type plants (FIG. 18A). The phenotypes observed in these lines are stable and have been observed for 3 generations.

Generation of PERK1 B. napus Transgenic Plants

Transgenic Brassica napus (cultivar Westar) plants were also generated harboring the sense PERK1 cDNA under the control of the 35S CaMV promoter cloned into the pCAMBIA 2301 plant transformation vector using Agrobacterium tumefaciens (strain GV3101 pmp90) which mediated the transformation of Brassica napus cotyledons. Kanamycin resistant plants have been selected. These transgenic plants flower at an earlier age than wild type plants.

PERK1 is a Plasma Membrane Associated Protein

The prediction that PERK1 is a receptor kinase also shows that PERK1 is a membrane-associated protein. We have demonstrated that PERK1 is localized to the membrane by generating a PERK1-GFP (green fluorescence protein) fusion construct under the control of the CaMV 35S promoter. This PERK1-GFP expression vector was then transformed into onion epidermal cells by biolistic bombardment and the localization of PERK1 was determined by GFP fluorescence. While the GFP control was found throughout the cell, PERK1-GFP was strictly localized to the periphery of the cell corresponding to the plasma membrane (FIG. 19).

### EXAMPLES

Isolation and Sequence Analyses of PERK1 cDNA

In order to isolate novel receptor-like protein kinases in B. napus a combination of degenerate oligonucleotide primers designed against conserved kinase subdomains I and VII (Hanks and Quinn, 1991) were used to amplify mass excised phagemid DNA from a newly constructed lambda-pistil cDNA library. The cDNAs encoding products of the expected length (˜420-450 bp) were cloned and the deduced partial sequences were analysed against several databases in order to determine which clone represented a novel kinase. One of several candidates, showed the highest degree of sequence similarity to the cytoplasmic domain of known plant receptor protein kinases, and was therefore used to screen the amplified lambda-pistil cDNA library. Several positive clones obtained from the library screen were completely sequenced and a partial 1512 bp consensus sequence was generated to represent the PERK1 cDNA isolated from the library screen. Although this partial PERK1 cDNA had an open reading frame, it did not encode a full length transcript, therefore the 5′ end was completed by 5′ RACE (see Methods).

The deduced amino acid sequence of PERK1 is shown in FIG. 1A and a schematic representation of its hydropathy plot is shown in FIG. 1B. The full length cDNA sequence is 2189 bp and consists of one large open reading frame of 1944 bp encoding a predicted protein of 648 amino acids with an estimated molecular mass of 69 kDa (FIG. 1). The first methionine of this open reading frame is preceded by two in frame stop codons, TAA and TGA at positions −48 to −45 and −23 to −21 respectively. In addition, there is also an AGAA sequence at position −9 to −6 (FIG. 1) which is a favourable site for translation initiation in all eukaryotes (Lutcke et al., 1987).

PERK1 encodes a receptor-like kinase possessing an extracellular domain, a single membrane spanning domain and an intracellular kinase domain (FIG. 1B) with four potential N-linked glycosylation sites (Asn-X-Ser/Thr) found throughout the sequence (FIG. 1A) (Weinstein et al., 1982). The predicted polypeptide sequence was analyzed using the PSORT database and determined to be a Type Ib intergral membrane protein with a hydrophilic amino terminal domain exposed on the exterior of the membrane but whose coding sequence does not indicate a cleavable signal sequence preceding this domain. Singer (1990) proposes that despite the lack of a signal peptide, Type Ib integral membrane proteins are inserted into the membrane via the usual ER-translocator protein machinery with some slight modifications. We have confirmed this by showing that the PERK1-GFP fusion is localized to the plasma membrane (FIG. 19). The extracellular domain of this protein consists of 137 amino acids (FIG. 1A) rich in proline and sharing sequence similarity with extensins, a family of hydroxyproline-rich glycoproteins (HRGPs) that constitute a major protein component of higher plant cell walls (Showalter, 1993). Extensin proteins have two proposed functions in plants, one which contributes to the structural support of the cell wall by forming glycoprotein networks and the other which involves plant defense; helping to protect the plant against mechanical wounding or pathogen attack (Wilson and Fry, 1986; Showlater, 1993). A distinctive characteristic prevalent among dicot extensins is the repetitive Ser-(Pro)₄ pentapeptide consensus motif (Showalter, 1993). A unique feature of PERK1 's extracellular domain is the predominance of a slightly modified Ser-(Pro)₂₋₃ motif in addition to the presence of one signature pentapeptide block (FIG. 1A). In order to investigate the phylogenetic status of PERK1, sixty four deduced amino sequences corresponding to the extracellular and transmembrane domains of extensin, proline rich and other cell wall proteins were retrieved from Genbank and used to construct a phylogenetic tree (Clustal X). Results from the phylogenetic analysis indicated that PERK1 is most similar to a subset of extensin proteins as shown by the sequence homology restricted predominantly to the serine/proline rich regions of the protein (data not shown). Extensins and proline rich proteins comprise two major classes of cell wall proteins and are essential for maintaining the proper architecture of a plant cell wall as well as important in helping protect plant cells against wounding and pathogen invasion. These protein families have been the focus of many research efforts and members belonging to these protein classes have been isolated in a wide range of plant systems. A certain degree of sequence identity or conservation is retained among these proteins isolated from different plants. Given that PERK1 shows homology in its extracellular domain to the extensin family of cell wall proteins and is rich in proline residues, homologues of PERK1 exist in many other plant systems.

The protein also contains two other domains of note. Hydropathy analysis (Kyte and Doolittle, 1982) of the protein sequence predicted a membrane spanning region of 23 amino acids (FIG. 1; residues 138-160) followed by a characteristic stop transfer sequence rich in charged amino acids [Arg-Arg-Arg] required for the proper insertion in the membrane (Weinstein et al., 1982).

All known protein kinases display amino acid sequence similarity in their catalytic domains which are comprised of eleven subdomains containing some invariant residues important for catalysis (Hanks and Quinn, 1991). The overall features of this organization are identified in the catalytic domain of the PERK1 protein in that all of the absolutely conserved amino acids as well as the highly conserved amino acid groups are present (FIG. 1A). The sequences of DIKASN in subdomain VI and GTFGYLAPE in subdomain VIII (Hanks and Quinn, 1991) are strong indicators that PERK1 may possess serine/threonine rather than tyrosine substrate specificity (FIG. 1A).

PERK1 is a Single Copy Gene and Ubiquitously Expressed in B. napus Tissue

As shown in FIG. 2, Southern blot analysis was performed under conditions of varying stringency using B. napus genomic DNA digested with several restriction enzymes in order to determine copy number of PERK1 in the Brassica genome. Based on known restriction sites within the cDNA and identical hybridization patterns obtained for low and high stringency conditions (FIG. 2), PERK1 exists as a single copy gene and is not a member of a multigene family.

In order to determine whether PERK1 is expressed in plant tissues, RNA gel blot analysis was performed using total RNA isolated from a variety of B. napus tissues as shown in FIG. 3. The full length PERK1 cDNA probe used in this experiment detected a transcript of 2.2 kb (FIG. 3; upper panel) which is consistent with the size of the full length PERK1 cDNA. The 2.2 kb PERK1 transcript was most abundant in B. napus, stem petal and pistil tissue (FIG. 3; upper panel). Levels of PERK1 mRNA were also detected in root, leaf and anther tissues albeit at much lower levels (FIG. 3; upper panel). As an internal control the blot was reprobed with a 18S rRNA to ensure even loading of the total RNA. A transcript detected with relatively the same intensity in all tissues indicates that equal amounts of total RNA was used (FIG. 3; lower panel). \ The difference in the intensity of the 18S rRNA signal in anther tissue (FIG. 3; lower panel) is a common problem associated with the desiccate nature of this tissue.

Changes in PERK1 mRNA in Response to Wounding and Chemical Elicitors

In order to examine whether PERK1 expression could be influenced by external stimuli, leaf and stem tissue of B. napus plants were wounded and the abundance of PERK1 mRNA was determined by standard Northern blot analysis using the full length PERK1 cDNA as a probe (see Methods). FIG. 4 shows changes in the steady-state levels of PERK1 mRNA accumulation following injury. PERK1 transcripts in wounded leaf tissue began to accumulate 5 min after wounding, reaching maximal levels within 15 min post injury represented by an 12fold induction. A 4.5 fold increase in PERK1 mRNA levels was detected 45 min following treatment declining towards basal levels by 2 hr (FIG. 4).

A similar profile of PERK1 mRNA steady state levels was obtained for wounded stem tissue (FIG. 4). An accumulation of PERK1 mRNA in stem is evident 5 min following wounding which represents a 3.6 fold induction of this gene. Maximum steady state levels of PERK1 mRNA in stem was achieved 30 min after injury corresponding to a 7 fold induction. PERK1 mRNA levels also accumulate in wounded leaf disc tissue as well as following an abrasive wounding treatment (FIGS. 4a, 4b). Levels of PERK1 mRNA also increase rapidly in the roots of hydroponically grown Brassica napus plants (FIG. 4c). Therefore, the overall kinetics of PERK1 mRNA accumulation in leaf, stem and root tissue after mechanical wounding is clearly a very rapid response (FIG. 4).

Defense mechanisms deployed by plants in response to wounding or pathogen attack have been shown to be induced by certain plant derived chemicals such as methyl jasmonate (MeJA) and salicylic acid (SA). In order to examine changes in the levels of PERK1 mRNA abundance in response to exogenous application of MeJA, B. napus plants were thoroughly sprayed with a 5 micromolar MeJA solution. Leaf and stem tissue was subsequently harvested at various times and the steady state levels of PERK1 mRNA were analysed. FIG. 5A shows the RNA gel blot and corrected PERK1 mRNA profile for treated leaf tissue during which no significant accumulation of PERK1 mRNA was detected. In response to MeJA, levels of PERK1 transcript in leaf tissue were very weak resembling basal levels in untreated tissue (FIG. 3; upper panel). Exogenous application of MeJA to stem tissue had no effect on the accumulation of PERK1 mRNA as shown by the corrected profile in which the fold induction of PERK1 did not deviate substantially from the untreated control (0 hr) (FIG. 5B). Furthermore, no increase in the steady state levels of PERK1 mRNA was detected in the appropriate control treatment (0.1% [v/v ] ethanol, solvent control for MeJA) at time 0 hr.

Many genes isolated to date that are induced by a pathogenic stimulus can be at least partially induced by SA (Ward et al., 1991). In order to address the potential role of PERK1 in a plant's defense response against pathogen attack, 4 mM SA was used as a chemical elicitor and sprayed onto B. napus plants. FIG. 6A shows that when SA is exogenously applied to leaf tissue, PERK1 mRNA accumulates 15 min following treatment reaching a maximum 5 fold induction 45 min post-treatment. Steady state levels of PERK1 mRNA in treated stem tissue peaked at 45 min corresponding to an approximate 2 fold induction in response to 4 mM SA (FIG. 6B).

In order to address whether levels of PERK1 mRNA accumulate in response to a pathogen attack, Brassica napus leaf tissue was inoculated with the fungal pathogen Sclerotinia sclerotiorum (FIG. 7). A 2.5 fold induction in the levels of PERK1 mRNA was detected 10 hrs following inoculation with the fungus. This evidence suggests that PERK1 may play a role in mediating a plant's defense response against pathogen attack.

Materials and Methods

Construction of Lambda-Pistil cDNA Library

Pistils were collected from floral buds of Westar and W1 cultivars 1-2 days before anthesis. Total RNA was isolated using the method described by Jones et al. (1985), and enriched for poly(A)⁺ mRNA by affinity chromatography using pre-packed oligo (dT)₂₅-cellulose beads (New England Biolabs, Beverly, Mass.). Approximately five micrograms of pistil poly(A)⁺ mRNA was used for the construction of a cDNA library using the ZAP-cDNA® synthesis kit (Stratagene, La Jolla, Calif.). The information encoded by the poly(A)⁺ mRNA was reversed transcribed using M-MuLV RT and converted into stable, unidirectional cDNA which was subsequently inserted into a self-replicating Uni-ZAP XR vector, packaged into phage particles in three separate packaging reactions and amplified as described by the manufacturer's procedures (Stratagene, La Jolla, Calif.). Infection of Escherichia coli host strain XLI-Blue yielded a primary library with an average titer of 1.0×10⁶ plaque forming units. The primary library was subsequently amplified to obtain an average total of 6.6×10¹⁰ plaque forming units.

Generation of Novel Receptor-Like Protein Kinase Clones

The isolation of novel Brassica napus receptor kinases relied upon the newly constructedcDNA library and involved in vivo mass excision of the pBluecsript phagemids from the Uni-ZAP XR vectors as outlined by the manufacturer (Stratagene, La Jolla, Calif.). Following efficient mass excision, phagemid DNA was extracted using a large scale alkaline protocol as described by Sambrook et al. (1989) and subjected to the polymerase chain reaction (PCR) using two separate oligonucleotide combinations, RK1/RK2 and RK1/RK3 (obtained from M. Cock, École Normale Supérieure de Lyon, France) specifically designed to prime conserved subdomains of the catalytic domain of receptor protein kinases. RK1 (5-ggiggTTTCggiAT^{T}c_{A}gTiTT^{A}_{T}^{T}_{C}AA^{A}_{g}gg-3′) served as the forward primer and was constructed based upon a conserved amino acid consensus (GGFGIV^{F}/_{Y}KG) within subdomain I of the catalytic domain. The degeneracy of one reverse primer RK2 (5′-AAiATiC^{T}_{g}igCCATiCC^{A}_{g}AA^{A}_{g}^{T}_{C}-3′) reflects a conserved amino acid consensus (DFGMARIF) of subdomain VII which closely resembles the SRKs in Brassica. The second reverse oligonucleotide RK3 (5′-A^{g}_{A}iA^{g}_{A}^{T}_{C}TTigCiA^{A}_{g}iCC^{A}_{g}AA^{A}_{g}TC-3′) was generated based upon conserved amino acids (DFGLAKLL) within subdomain VII prevalent among the RLKs isolated in Arabidopsis. Phagemid DNA was amplified in a reaction mixture containing 1 microliter of excised phagemid DNA, 10× PCR buffer (100 mM Tris-HCl pH 8.3, 500 mM KCl, 15 mM MgCl₂), 10 mM deoxyribonucleotide triphosphate mixture, 10 micromolar of each oligonucleotide primer and 0.5 microliter Tsg polymerase (BioBasics, Canada). The PCR reaction was heated at 95° C. for 2 min and amplified for 35 cycles under the following amplification conditions: 1 min at 95° C. for denaturation, 1 min 30 sec at 50° C. for primer annealing and 1 min at 72° C. for synthesis. A final extension cycle of 10 min at 72° C. was also incorporated into the amplification program. All PCR products generated of the expected size (420-450 bp) were gel purified, cloned into the pT7Blue plasmid (Novagen, Madison, Wis.) and introduced into Escherichia coli DH5-alpha. Transformants were tested for the presence of an insert and positive clones were sequenced with universal primers (R-20 and U-19) by an ABI automated sequencer (Model 373 STRETCH DNA; Perkin Elmer Corp., Canada Ltd.) using the dideoxychain-terminating method described by Sanger et al. (1977). Sequence analyses performed using DNAsis® software (Hitachi Software, San Bruno, Calif.) at the nucleotide and amino acid levels.

Screening of Lambda-Pistil cDNA Library

The original 351 bp PCR product was used to screen the lambda-pistil cDNA library. Approximately 2×10⁶ plaques from the amplified library were screened and plated at a density of 1×10⁵ pfu/plate. Duplicate colony lifts were performed according to Sambrook et al. (1989), and prehybridized for 2 hr at 42° C. in 50% formamide, 5× Denhardt's solution (1× Denhardt's solution is 0.02% Ficoll, 0.02% DVP, 0.02% BSA), 5× SSC (1× SSC is 0.15M NaCl, 0.015M sodium citrate), 0.1% SDS, 1 mM EDTA and 100 μg/ml salmon sperm DNA. Filters were subsequently hybridized overnight in the same solution containing the 351 bp PERK1 cDNA radiolabeled by random priming (Feinberg and Vogelstein, 1983) and washed twice with 2× SSC, 0.1% SDS at room temperature for 15 min, followed by two 25 min washes with 0.5× SSC, 0.1% SDS at 55° C. Plaques containing putative positive clones were cored and subjected to several rounds of screening until single isolates representing the PERK1 clone were obtained. Single clone excision to liberate the double stranded pBluescript phagemid was performed on each isolate according to the procedure recommended by the manufacturer (Stratagene, La Jolla, Calif.). Phagemid DNA digested with EcoRI/XhoI to release the cloned cDNA was subjected to standard plasmid Southern blot analysis as described by Sambrook et al. (1989) and probed with the radiolabeled 351 bp PERK1 cDNA. The membrane was prehybridized at 42° C. in 5× SSPE, 10× Denhardt's solution and 0.5% SDS for 2 hr and hybridized overnight at the same temperature in a buffer containing 50% formamide, 5× SSPE and 0.5% SDS. Washing conditions were performed twice at room temperature for 15 min in 2× SSC, 0.1% SDS followed by several 30 min washes at 55-60° C. in 0.1× SSC, 0.1% SDS. An intense hybridization signal would confirm whether phagemids isolated from the library screen contained the cloned cDNA of interest. Several positive clones were sequenced as previously mentioned using both universal and sequence specific primers to generate a consensus sequence representing the PERK1 cDNA clone (1512 bp) isolated from the lambda-pistil cDNA library.

Rapid Amplification of cDNA Ends (5′RACE)

The 5′ end of the PERK1 cDNA was obtained by the procedure for the rapid amplification of cDNA ends originally described by Frohman et al. (1988) using the 5′ RACE System, Version 2.0 kit (Gibco-BRL, Gainthersburg, Md.). First strand cDNA was synthesized from approximately 300 μg of mixed Westar and W1 pistil total RNA using a gene specific primer GSP1 (5′-TAACCAACAAgAgACA-3′) designed to anneal approximately 300 bp from the 5′ end of the PERK1 cDNA (1512 bp) isolated from the library screen. Following cDNA synthesis, the first strand product was purified from unincorporated dNTPs and GSP1 using a GLASS MAX® spin cartridge. A homopolymeric tail was added to the 3′ end of the cDNA using TdT (terminal deoxynucleotidyl transferase) and dCTP. Tailed cDNA was amplified using a second gene specific primer GSP2 (5′-CCACTCCCAACTTTCAAC-3′) designed to anneal 3′ to GSP1 with respect to the cDNA, and an abridged anchor primer (Gibco-BRL, Gainthersburg, Md.) which annealed to the homopolymeric tail. PCR amplification was carried out for 35 cycles of denaturation at 94° C. for 1 min, primer annealing at 55° C. for 1 min and extension at 72° C. for 2 min, followed by a final extension cycle for 10 min. A PCR product of the expected size (˜1 kb) corresponding to the 5′ end of PERK1 was gel purified, cloned into the pT7Blue plasmid (Novagen, Madison, Wis.) and transformed into Escherichia coli DH5-alpha. Confirmation of the 5′RACE product was obtained by plasmid Southern blot analysis as described above and by sequential primer based sequencing.

Cloning of Full Length PERK1 cDNA

A PCR based approach was used to generate a full length PERK1 cDNA by combining the 5′RACE product cloned into the EcoRV site of pT7Blue with the cDNA isolated from the library screen cloned into the EcoRI/XhoI sites of the pBluescript SK phagemid. A forward primer (5′-ggAAAgCTTgCATgCCTgCAggTCgAC-3′) containing an internal PstI site was designed to anneal upstream to the EcoRV cloning site of pT7Blue. A reverse primer (5′-CgCCTgCAggTAATACgACTCACTATAggg-3′) also containing a PstI site was designed based on pBluecsript phagemid sequence immediately 3′ to the EcoRI/XhoI cloning site. Full length PERK1 cDNA was generated from a 100 microliter PCR reaction containing 1 microliter (˜20 ng) of each template (cDNA in pT7Blue and pBluescript phagemid), 10× Pfu Buffer (200 mM Tris-HCl pH8.8, 100 mM (NH₄)₂SO₄, 20 mM MgSO₄, 1% Triton®X-100, 1 mg/mlBSA), 10 mM dNTPs, 50 pmol forward and reverse primers and 1 microliter Pfu polymerase (Gibco-BRL, Gainthersburg, Md.). The samples were heated to 94° C. for 5 min and amplified for 30 cycles with a denaturing cycle of 1 min, a pimer annealing cycle at 53° C. for 1 min followed by an extension cycle for 3 min at 72° C. The resulting PCR product of the expected size (˜2.2 kb) was gel purified and cloned into the PstI restriction site of pBluescript KS (±) II. The full length PERK1 cDNA sequence was confirmed by a sequential primer based sequencing approach using both universal and sequence specific primers as previously described. All DNA and protein sequence analysis was performed using the DNAsis® Software (Hitachi Software, San Bruno, Calif.).

Genomic DNA Isolation and Southern Blot Analysis

Genomic DNA was extracted from approximately one gram of young Brassica napus leaf tissue according to the method described by Goring et al. (1992b). Approximately 5 μg of genomic DNA was digested with several restriction enzymes (BamHI, EcoRI, HindIII, PstI, XbaI, XhoI), fractionated through a 0.8% agarose gel and transferred overnight in 10× SSC onto Zetaprobe membrane (Biorad, Hercules, Calif.). This was performed in duplicate to test hybridization conditions under low and high stringencies conditions. After drying, the membranes were prewashed in 0.1× SSC, 0.5% SDS for 25 min at 60° C. The membranes were prehybridized and hybridized as previously described for plasmid Southern blots with the inclusion of 10% dextran sulfate and 50 microgram/milliliter salmon sperm DNA in the hybridization buffer. Washing conditions for genomic southern blots varied depending on the stringency tested. One membrane was washed under conditions of low stringency for 15 min at room temperature in 2× SSC, 0.1% SDS followed by second 15 min room temperature wash in 1× SSC, 0.1% SDS and three final washes at 50° C. in 1× SSC, 0.1% SDS. The second membrane was washed under conditions of high stringency by lowering the salt concentration to 0.1× SSC, 0.1% SDS and increasing the temperature to 65° C. The ³²P-labeled 1512 bp PERK1 cDNA probe was generated by random priming as described by Feinberg and Vogelstein (1983). Membranes were subjected to autoradiography (XAR-5 film, Kodak) overnight at −80° C.

Isolation and Northern Blot Analysis of Multiple Tissue RNA

Total RNA was extracted from a mixture of Westar and W1 root, stem, leaf, petal, anther and pistil tissue as described by Jones et al. (1985). Approximately 40 micrograms of total RNA was fractionated on a 1.2% formaldehyde gel (Sambrook et al., 1989) and transferred to Zetaprobe membrane (Biorad, Hercules, Calif.) in 10× SSC. Hybridization and high stringency wash conditions were conducted as previously described for genomic Southern blot analysis. The membrane was subsequently probed with a 18SrRNA as an internal control for even loading of total RNA.

Plant Treatments

B. napus plants were grown in a growth chamber at 22° C. with a 16 hr light period followed by an 8 hr dark period at 16° C. Experiments were conducted on two month old plants, and all experiments used one plant per time point from which leaf and stem tissue was harvested.

Wounding of leaf material was performed by punching out leaf discs every 1 cm around the perimeter of the leaf blade ensuring that the midvein remained intact, and stems were wounded by slicing into 1-3 cm segments. The wounded tissues were placed in petri dishes containing filter paper moistened with 20 mM sodium phosphate buffer supplemented with 50 microgram/milliliter chloramphenicol to prevent bacterial contamination of the wounded tissue (Shirsat et al., 1996). A control (0 hr) time point for this experiment was performed by incubating unwounded tissue in the sodium phosphate buffer. Wounded leaf and stem tissue was harvested at different times after wounding (0 hr, 5 min, 15 min, 45 min, 1 hr, 4 hr, 12 hr, 24 hr, 36 hr, 48 hr). The procedures for the other wounding treatments assayed are described in detail in the figure legends (FIGS. 4a-c).

Plants were thoroughly sprayed with 50 micromolar methyl jasmonate (MeJA; Sigma, St. Louis, Mo.) (Titarenko et al., 1997) and 4 mM salicylic acid (SA; Sigma, St. Louis, Mo.) solutions (Schweizer et al., 1998). Leaf and stem tissue was harvested at various time points (0 hr, 5 min, 15 min, 30 min, 45 min, 1 hr, 2 hr, 4 hr, 12 hr, 24 hr, 36 hr, 48 hr, 72 hr, 96 hr) following SA and MeJA treatments. Control sprays were performed with the carrying solutions in the absence of the chemical inducer. Carrying solutions were 5 mM phosphate buffer, pH 7 for SA, and 0.1% [v/v ] ethanol for MeJA. The method of inoculation used for the fugal treatment is described in the figure caption for FIG. 7.

Total RNA was extracted from treated tissue according to the method described by Cock et al. (1997). Depending on the treatment, varying amounts of total RNA (20-40 micrograms) was electrophoresed on a 1.2% formaldehyde and standard Northern blot analysis was performed as described by Sambrook et al. (1989). Hybridization and washing conditions were performed as outlined for the multiple tissue northern blot. Following autoradiography, the amounts of radioactive signal were quantified using Instant Imager Electronic Autoradiography (Packard, Meriden, Conn.). The membranes were reprobed with the cyclohpilin cDNA and the amounts of hybridized radiolabeled cyclophilin were quantified in the same manner. The relative amounts of RNA hybridized to the full length PERK1 cDNA probe were determined after correction for differences in the amounts of cyclophilin RNA.

The present invention has been described in detail and with particular reference to the preferred embodiments; however, it will be understood by one having ordinary skill in the art that changes can be made thereto without departing from the spirit and scope of the invention.

All articles, patents and other documents described in this application (including database sequences and/or accession numbers), U.S. patent application Ser. No. 60/149,466 filed on Aug. 19, 1999 (entitled “Characterization of a Novel Receptor Kinase from Brassica with a Putative Role in Plant Defense”), U.S. patent application Ser. No. 60/159,122 and U.S. Pat. Nos. 5,612,191, 5,763,211, 5,750,848 and 5681714, are incorporated by reference in their entirety to the same extent as if each individual publication, patent or document was specifically and individually indicated to be incorporated by reference in its entirety. They are also incorporated to the extent that they supplement, explain, provide a background for, or teach methodology, techniques and/or compositions employed herein.

### REFERENCES

Braun, D. M., and Walker, J. C. (1996). Plant transmembrane receptors: new pieces in the signalling puzzle. TIBS 21, 70-73.

Becraft, P. W., Stinard, P. S. and McCarty, D. R. 1996. CRINKLY4: a TNFR-like receptor kinase involved in maize epidermal differentiation. Science 273: 1406-1409.

Braun, D. M. and Walker, J. C. 1996. Plant transmembrane receptors: new pieces in the signalling puzzle. TIBS 21: 70-73.

Cassab, G. I. (1998). Plant cell wall proteins. Annu. Rev. Plant Physiol. Plant Mol. Biol. 49, 281-309.

Chang, C., Schaller, G. E., Patterson, S. E., Kwok, S. F., Meyeroxitz, E. M., and Bleeker, A. B. (1992). The TMK1 gene from Arabidopsis codes for a protein with structural and biochemical characteristics of a receptor protein kinase. Plant Cell 4, 1263-1271.

Clark, S., Williams, R. W., and Meyerowitz, E. M. (1997). The CLAVATA1 gene encodes a putative receptor-kinase that controls shoot and floral meristem size in Arabidopsis. Cell 89, 575-585.

Cock, J. M., Swarup, R., and Dumas, C. (1997). Natural antisense transcripts of the S locus receptor kinase gene and related sequences in Brasscia oleracea. Mol. Gen. Gent. 255, 514-524.

Corbin, D. R., Sauer, N., Lamb, C. J. (1987). Differential regulation of a hydroxyproline-rich glycoprotein gene family in wounded and infected plants. Mol. Cell Biol. 7, 4337-4344.

Doares, S. H., Narváez-Vásquez, J., Conconi, A., and Ryan, C. A. (1995). Salicylic acid inhibits synthesis of proteinase inhibitors in tomato leaves inducted by systemin and jasmonic acid. Plant Physiol. 108, 1741-1746.

Dwyer, K. G., Kandasamy, M. K., Mahosky, D. I. Acciai, J., Kudish, B. I. Miller, J. E., Nasrallah, M. E., and Nasrallah, J. B. (1994). A superfamily of S locus-related sequences in Arabidopsis: Diverse structures and expression patterns. Plant Cell 6, 1829-1843.

Feinberg, A. P. and Vogelstein, B. (1983). A technique for radiolabeling DNA restriction endonuclease fragments to high specific activity. Anal. Biochem. 132, 6-13.

Frohman, M. A., Dush, M. K., and Martin, G. R. (1988). Rapid production of full length DNAs from rare transcripts: Amplification using a single gene-specific oligonucleotide primer. Proc. Natl. Acad. Sci. USA 85, 8998-9002.

Goring, D. R., and Rothstein, S. J. (1992). The S-locus receptor kinase gene in a self-incompatible Brassica napus line encodes a functional serine/threonine kinase. Plant Cell 4, 1273-1281.

Goring, D. R., Banks, P., Fallis, L., Baszczynski, C. L., Beversdorf, W. D., and Rothstein, S. J. (1992b). Identification of an S-locus glycoprotein allele introgressed form B. napus ssp. oleifera. Plant J. 2, 983-989.

Hanks, S. K., and Quinn, A. M. (1991). Protein kinase catalytic domain sequence database: Identification of conserved features of 1° structure and classification of family members. Methods Enzymol. 200, 38-62.

He, Z. H., Fujuki, M. and Kohorn, B. D. 1996. A cell wall-associated, receptor-like kinase. J. Biol. Chem. 271: 19789-19793.

He, Z. H., He, D. and Kohorn, B. D. 1998. Requirement for the induced expression of a cell wall-associated receptor kinase for survival during the pathogen response. Plant J. 14: 55-63.

He, Z. H., Cheeseman, I., He, D. and Kohorn, B. D. 1999. A cluster of five cell wall-associated receptor kinase genes, Wak1-5, are expressed in specific organs of Arabidopsis. Plant Mol. Biol. 39: 1189-1196.

Hervé, C., Dabos, P. Galaud, J. -P. Rouge, P., and Lescure, B. (1996). Characterization of an Arabidopsis thaliana gene that defines a new class of putative plant receptor kinases with an extracellular lectin-like domain. J. Mol. Biol. 258, 778-788.

Horn, M. A., and Walker, J. C. (1994). Biochemical properties of the autophosphorylation of RLK5, a receptor-like protein kinase from Arabidopsis thaliana. Biochem. et Biophys. Acta 1208, 65-74.

Jinn, T. L., Stone, J. M. and Walker, J. C. 2000. HAESA, an Arabidopsis leucine-rich repeat receptor kinase, controls floral organ abscission. Genes Dev. 14: 108-17.

Jones, J. D. G., Dunsmuir, P., and Bedbrook, J. (1985). High level expression of introduced chimeric genes in regenerated transformed plants. EMBO 4, 2411-2418.

Keller, B., and Lamb, C. J. (1989). Specific expression of a novel cell wall hydroxyproline-rich glycoprotein gene in lateral root initiation. Genes Dev. 3, 1639-1646.

Kohorn, B. D., Lane, S., and Smith, T. A. (1992). An Arabidopsis serine/threonine kinase homologue with an epidermal growth factor repeat selected in yeast for its specificity for a thylakoid membrane protein. Proc. Natl. Acad. Sci. USA 89, 10989-10992.

Kyte, J., and Doolittle, R. F. (1982). A simple method for displaying the hydropathic character of a protein. J. Mol. Biol. 157, 105-132.

Lally, D., Ingmire, P., Tong, H. Y. and He Z. H. 2001. Antisense expression of a cell wall-associated protein kinase, WAK4, inhibits cell elongation and alters morphology. Plant Cell 13: 1317-1331.

Lease, K., Ingham, E. and Walker, J. C. 1998. Challenges in understanding RLK function. Curr. Opin. Plant Biol. 1: 388-392.

Leyser O (2001) Auxin signaling: the beginning, the middle and the end. Curr. Opin. Plant Biol. 4: 382-386.

Li, J., and Chroy, J. (1997). A putative leucine rich repeat receptor kinase involved in brassinosteroid signal transduction. Cell 90, 929-938.

Lin, H. V., Wang, X. F., Ng-Eaton, E., Weinberg, R. A., and Lodish, H. F. (1992). Expression cloning of TGF-type II receptor, a functional transmembrane serine/threonine kinase. Cell 68, 775-785.

Lutcke, H. A., Chow, K. C., Mickel, F. S., Moss, K. A., Kern, H. F., and Scheele, G. A. (1987). Selection of AUG initiation codons differs in plants and animals. EMBO J. 6, 43-48.

McCarty, D. R. and Chory, J. 2000. Conservation and innovation in plant signalling pathways. Cell 103: 201-209.

Merkouropoulos, G., Barnett, D. C., and Shirsat, A. H. (1999). The Arabidopsis extensin gene is developmentally regulated, is induced by wounding, methyl jasmonate, abscissa acid and salicylic acid, and coded for a protein with unusual motifs. Planta 208, 212-219.

Mockaitis K, and Howell S H (2000) Auxin induces mitogenic activated protein kinase (MAPK) activation in roots of Arabidopsis seedlings. Plant J. 24, 785-796.

Mu, J. -H., Lee, H. -S., and Kao, T. (1194). Characterization of a pollen expressed receptor-like kinase gene of Petunia inflata and the activity of its encoded kinase. Plant Cell 6, 709-721.

Nasrallah, J. B., and Nasrallah, M. E. (1993). Pollen-stigma signaling in the sporophytic self-incompatibility response. Plant Cell 5, 1325-1335.

Penninckx, I. A. M., Eggermont, K., Terras, F. R. G., Thomma, B. P. Samblanx, G. W. Buchala, A., Métraux, J. P., Manners, J. M. and Broekaert, W. F. (1996). Pathogen-induced systemic activation of a plant defensin gene in Arabidopsis follows a salicylic acid-independent pathway. Plant Cell 8, 2309-2323.

Peña-Cortés, H., Albercht, T., Prat, S., Weiler, E. W., and Willmitzer, L. (1993). Aspirin prevents wound-induced gene expression in tomato leaves by jasmonic acid biosynthesis. Planta 191, 123-128.

Reed J W (2001) Roles and activities of Aux/IAA proteins in Arabidopsis. Trends in Plant Science. 6(9): 420-425.

Sambrook, J., Fritsch, E. F. and Maniatis, T. (1989). Molecular cloning: A Laboratory Manual, 2nd ed. New York: Cold Spring Harbor Laboratory Press.

Sanger, F., Nicklen, S., Coulson, A. R. (1977). DNA sequencing with chain terminating inhibitors. Proc. Natl. Acad. Sci. USA 74, 5463-5467.

Sauer, N., Corbin, D. R., Keller, B., and Lamb, C. J. (1990). Cloning and characterization of a wound specific hydroxyproline-rich glycoprotein in Phaseolus vulgarism Plant Cell Environ. 13, 257-266.

Schweizer, P., Buchala, A., Dudker, R., and Métraux, J. P. (1998). Induced systemic resistance in wounded rice plants. Plant J. 14, 475-481.

Shirsat, A. H., Wieczoreky, D., and Kozibal, P. (1996). A gene for Brassica napus extensin is differentially expressed on wounding. Plant Mol. Biol. 30, 1291-1300.

Shiu, S. H. and Bleecker, A. B. 2001. Receptor-like kinases from Arabidopsis form a monophyletic gene family related to animal receptor kinases. Proc. Natl. Acad. Sci. USA 98: 10763-10768.

Showalter, A. M., Zhou, J., Rumeau, D., Worst, S. G., and Varner, J. E. (1991). Tomato extensin and extensin-like cDNAs: Stucture and expression in response to wounding. Plant Mol. Biol. 16, 547-565.

Showlater, A. M. (1993) Structure and function of plant cell wall proteins. Plant Cell 5, 9-23.

Silva, N. F., Stone, S. L., Christie, L. N., Sulaman, W., Nazarian, K. A., Burnett, L. A., Arnoldo, M. A., Rothstein, S. J. and Goring, D. R. 2001. Expression of the S receptor kinase in self-compatible Brassica napus cv. Westar leads to the allele-specific rejection of self-incompatible Brassica napus pollen. Mol. Genet. Genomics. 265: 552-559.

Singer, S. J. (1990). The structure and insertion of integral proteins in the membrane. Annu. Rev. Cell Biol. 6, 247-296.

Song, W. -Y., Wang, G. -L., Chen, L., Kim, H. -S., Pi, L. -Y., Gardner, J., Wang, B., Holsten, T., Zhai, W. -X., Zhu, L. -H., Fauquet, C., and Ronald, P. C. (1995). A receptor kinase-like protein encoded by the rice disease resistance gene Xa21. Science 270, 1804-1806.Stein, J. C., Howlett, B., Boyes, D. C., Nasrallah, M. E., and Nasrallah, J. B. (1991). Molecular cloning of a putative receptor protein kinase gene encoded at the self-incompatibility locus of Brassica oleracea. Proc. Natl. Acad. Sci. USA 88, 8816-8820.

Stone, J. M. and Walker, J. C. 1995. Plant protein kinase families and signal transduction. Plant Physiol. 108: 451-457.

Suzuki, K., and Shinshi, H. (1995). Transient activation and tyrosine phosphorylation of a protein kinase in tobacco cells treated with a fungal elicitor. Plant Cell 7, 639-647.

Swarup, R., Dumas, D., and Cock, J. M. (1996). A new class of receptor-like protein kinase gene from Arabidopsis thalaina possessing a domain with similarity of plant lectin genes. Plant Physiol. 111, 347.

Takasaki, T., Hatakeyama, K., Suzuki, G., Watanabe, M., Isogai, A. and Hinata, K. 2000. The S receptor kinase determines self-incompatibility in Brassica stigma. Nature 403: 913-916.

ten Dijke, P., Ichijo, H., Franzen, P., Schulz, P., Saras, J., Toyoshima, H., Heldin, C., and Miyazono, K. (1993). Activin receptor-like kinases: A novel subclass of cell surface receptors with predicted serine/threonine kinase activity. Oncogene 8, 2879-2887.

Titarenko, E., Rojo, E., Leon, J., and Sanchez-Serrano, J. (1997). Jasmonic acid-dependent and -independent signalling pathways control wound-induced gene activation in Arabidopsis thaliana. Plant Physiol. 115, 817-826.

Tobias, C. M., Howlett, B., and Nasrallah, J. B. (1992). An Arabidopsis thaliana gene with sequence similarity to the S-locus receptor kinase of Brassica oleracea. Plant Physiol. 99, 284-290.

Tobias, C. M. and Nasrallah, J. B. 1996. An S-locus-related gene in Arabidopsis encodes a functional kinase and produces two classes of transcripts. Plant J. 10: 523-31.

Tobias, K. U., Mitsukawa, N., Oosumi, T., Matsuura, Y., Yokoyama, R., Whittier, R. F. and Komeda, Y. (1996). The Arabidopsis ERECTA gene encodes a putative receptor protein kinase with extracellular leucine-rich repeats. Plant Cell 8, 735-746.

Trotochaud, A. E., Jeong, S. and Clark, S. E. 2000. CLAVATA3, a multimeric ligand for the CLAVATA1 receptor-kinase. Science 289: 613-617.

Truernit, E., Schmid, J., Epple, P., Illig, J., and Sauer, N. (1996). The sink specific stress-regulated Arabidopsis STP4 gene: Enhanced expression of a gene encoding a monosaccharide transporter by wounding, elicitors and pathogen challenge. Plant Cell 8, 2169-2182.

Ullrich, A., and Schlessinger, J. (1990). Signal transduction by receptors with tyrosine kinase activity. Cell 61, 203-212.

Usami, S., Banno, H. Ito, Y., Nishihama, R., and Machida, Y. (1995). Cutting activates a 46-kilodalton protein kinase in plants. Proc. Natl. Acad. Sci. USA 92, 8660-8664.

Wagner, T. A. and Kohorn, B. D. 2001. Wall-associated kinases are expressed throughout plant development and are required for cell expansion. Plant Cell 13: 303-318.Walker, J. C. (1993). Receptor-like protein kinase genes of Arabidopsis thaliana. Plant J. 3, 451-456.

Walker, J. C. (1994). Structure and function of the receptor-like protein kinases of higher plants. Plant Mol. Biol. 26, 1599-1609.

Walker, J. C., and Zhang, R. (1990). Relationship of a putative receptor protein kinase form maize tot he S-locus glycoproteins of Brassica. Nature 345, 743-746.

Wang, G. -L., Ruan, D. -L., Song, W. -Y., Sideris, S., Chen, L., Pi, L. -Y., Zhang, S., Zhang, Z., Fauquet, C., Gaut, B. S., Whaten, M. C., and Ronald, P. C. (1998). Xa21 encodes a receptor-like molecule with a leucine-rich repeat domain that determines race-specific recognition and is subject to adaptive evolution. Plant Cell 10, 765-779.

Wang, X., Zafian, P., Choudhary, M., and Lawton, M. (1996). The PR5K receptor protein kinase form Arabidopsis thaliana is structurally related to a family of plant defense proteins. Proc. Natl. Acad. Sci. USA 93, 2598-2602.

Ward, .R., Uknes, S. J., Williams, S. C., Dincher, S. S., Wiederhold, D. L., Alexander, D. C., Ahl-Goy, P., Métraux, J. -P., and Ryals, J. A. (1991). Coordinate gene activity in response to agents that induce systemic acquired resistance. Plant Cell 3, 1085-1094.

Weinstein, J. N., Blumenthal, R., Van Renwoude, J., Kempf, C., and Mauser, R. O. (1982). Charge clusters and the orientation of membrane proteins. J. Membr. Biol. 66, 203-212.

Wilson, L. G., and Fry, J. L. (1986). Extensins: A major cell wall glycoprotein. Plant Cell Environ. 9, 239-260.

Yang, Y., Shah, J., and Klessig, D. F. (1997). Signal perception and transduction in plant defense responses. Genes Dev. 11, 1621-1639.

Zhao, Y., Feng, X. -H., Watson, J. C., Bottino, P. J., and Kung, S. -D. (1994). Molecular cloning and biochemical characterization of a receptor-like serine/threonine kinase from rice. Plant Mol. Biol. 26, 791-803.

Zhou, J., Loh, Y. -T., Bressan, R. A., and Martin, G. B. (1995). The tomato gene Pti1encodes a serine/threonine kinase that is phosphorylated by Pto and is involved in the hypersensitive response. Cell 83, 925-935.

We claim:

1. A method for producing a transgenic plant having increased plant height, number of branches, number of seed pods and/or seed production compared to a non-transgenic plant, and/or quicker flowering or later senescence compared to a non-transgenic plant, comprising transforming a plant with a vector including a PERK nucleic acid molecule or a nucleic acid molecule having PERK activity.

2. A method of producing a genetically transformed plant which expresses a PERK polypeptide or a polypeptide having PERK activity, the plant having increased plant height, number of branches, number of seed pods and/or seed production, compared to wild-type plants, and/or quicker flowering or later senescence compared to a non-transgenic plant, comprising:
(a) cloning or synthesizing a PERK nucleic acid molecule or nucleic acid molecule having PERK activity;
(b) inserting the nucleic acid molecule in a vector so that the nucleic acid molecule is operably linked to a promoter;
(c) inserting the vector into a plant cell or plant seed;
(d) regenerating the plant from the plant cell or plant seed, wherein plant height, number of branches, number of seed pods and/or seed production compared to wild-type plants in the plant are increased or wherein the plant has quicker flowering or later senescence.

3. The method of claim 1 or 2, wherein the isolated nucleic acid molecule encoding a PERK polypeptide or polypeptide having PERK activity comprises a nucleic acid molecule selected from the group consisting of:
c) a nucleic acid molecule that hybridizes to a nucleic acid molecule consisting of all or part of [SEQ ID NO:1], or a complement thereof under low, moderate or high stringency hybridization conditions;
d) a nucleic acid molecule degenerate with respect to (a), wherein the nucleic molecule encodes a PERK polypeptide or a polypeptide having PERK activity.

4. The method of claim 1 or 2, wherein the nucleic acid molecule encoding a PERK polypeptide, or a polypeptide having PERK activity, comprises a nucleic acid molecule selected from the group consisting of:
(a) the nucleic acid molecule of the coding strand shown in [SEQ ID NO:1], or a complement thereof;
(b) a nucleic acid molecule encoding the same amino acid sequence as a nucleotide sequence of (a); and
(c) a nucleic acid molecule having at least 17% identity with the nucleotide sequence of (a) and which encodes a PERK polypeptide or a poypeptide having PERK activity

5. The method of claim 1 or 2, wherein the plant is of a species selected from the group consisting of alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rape, rice, roses, rye, salicornia sorghum, soybean, spinach, squash, strawberries, sunflower, sweet corn, tobacco, tomato and wheat.
